(19) United States
(12) Patent Application Publication
(10) Pub. No.: US 2010/0222269 A1
(43) Pub. Date: Sep. 2, 2010

(54) IMPROVED DERIVATIVES OF AMYLIN

(75) Inventors: Lauge Schäffer, Lyngby (DK); Thomas Kruse, Herlev (DK); Jesper Lau, Farum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/676,967

(22) PCT Filed: Sep. 11, 2008
(86) PCT No.: PCT/EP2008/062036

§ 371 Date: May 12, 2010

(30) Foreign Application Priority Data
Sep. 11, 2007 (EP) 07116067.5

(51) Int. Cl. A61K 38/22; C07K 14/435; A61P 3/10; A61P 3/04

(52) U.S. Cl. 514/12; 530/324

(57) ABSTRACT

The invention relates to derivatives of human amylin or analogues thereof which bind to the amylin receptor and are linked to an albumin binding compound, pharmaceutical compositions comprising these derivatives and methods for obtaining such.

### FIELD OF THE INVENTION

The invention relates to derivatives of human amylin or analogues thereof which bind to albumin and/or the amylin receptor, pharmaceutical compositions comprising these derivatives, methods for obtaining such and amylin derivatives for use as medicaments.

### BACKGROUND OF THE INVENTION

A large and growing number of people suffer from diabetes mellitus and obesity. Diabetes mellitus is a metabolic disorder in which the ability to utilize glucose is partly or completely lost.

A number of treatment regimes are targeting excessive blood glucose whereas others are focused primarily on weight reduction. The most efficient anti-diabetic agent used to lower blood glucose is insulin and analogue(s) thereof. It has been known for a long time that when traditional insulin is used to treat diabetes, it is associated with an increase in body weight. Insulin has to be injected subcutaneously up to several times per day.

Type 2 diabetes is generally treated in the early phases with diet and exercise. As the condition progresses, various oral anti-diabetic agents are added. Injected agents such as GLP-1 analogues may also be used at this stage. In general, these agents are most efficient in patients with functioning beta-cells capable of releasing insulin and amylin. Human amylin is a 37 amino acid long peptide which has physico-chemical properties that make its use as a drug troublesome. In particular, it has a tendency to fibrillate in-vitro and/or ex-vivo and become ineffective due to precipitation. A drug product called Symlin® is currently on the market which contains an analogue of human amylin (pramlintide). In pramlintide three of the 37 amino acids are substituted to proline compared to human amylin. This improves substantially the fibrillating tendency. However, even pramlintide is difficult to keep in solution at neutral pH and it is therefore provided in an acidic solution i.e. Symlin®.

Various amylin analogues and derivatives with improved pharmacokinetic or pharmacodynamic (PK/PD) properties have been described. However; there is still a need to provide analogues and derivatives that have the activities of native human amylin, as well as derivatives which have a protracted pharmacokinetic profile, show enhanced solubility and/or stability over native human amylin.

### SUMMARY OF THE INVENTION

The invention relates in one aspect to a derivative of human amylin or an analogue thereof, wherein
a) the amino acid residue in position 1 is any natural amino acid which is connected to a N-terminal extension consisting of 1-10 amino acids, wherein
   a. said extension is further linked to an albumin binding residue, optionally via a linker, or
   b. an amino acid residue in position 2-37 has been substituted with a lysine residue, and said lysine residue is linked to an albumin binding residue, optionally via a linker;
   or
b) an amino acid residue in position 3, 17, 21, 25 or 29 has been substituted with a lysine residue and wherein said lysine residue is linked to an albumin binding residue, optionally via a linker;
   or
c) the amino acid residue in position 18 is arginine, and an amino acid residue in position 1-17 or 19-37 has been substituted with a lysine residue, and wherein said lysine residue is linked to an albumin binding residue r, optionally via a linker;
   wherein the amino acid numbering conforms with the amino acid numbering in SEQ ID NO:17.

The invention relates in a further aspect to a method for increasing the time of action in a patient of human amylin or an analog thereof, characterized by modifying
a) the amino acid residue in position 1 by optionally substituting with any natural amino acid, and by connecting said natural amino acid to a N-terminal extension consisting of 1-10 amino acids, and further
   a. linking said extension to an albumin binding residue, optionally via a linker, or
   b. modifying an amino acid residue in position 2-37 by substitution with a lysine residue, and by linking said lysine residue to an albumin binding residue, optionally via a linker,
   or;
b) the amino acid residue in position 3, 17, 21, 25 or 29 by substitution with a lysine residue and by linking said lysine residue to an albumin binding residue, optionally via a linker,
   or;
c) the amino acid residue in position 18 by substitution with an arginine residue and an amino acid residue in position 2-17 or 19-37 by substitution with a lysine residue and by linking said lysine residue to an albumin binding residue, optionally via a linker;
d) the amino acid in position 18 is arginine, the amino acids in position 25, position 28 and position 29 are proline, and an amino acid residue in position 1-17 or 19-37 has been substituted with a lysine residue, and wherein such a lysine residue is linked to an albumin binding residue, optionally via a linker.
wherein the amino acid numbering conforms with the amino acid numbering in SEQ ID NO:17.

The present invention also provides pharmaceutical compositions comprising a derivative according to the present invention and the use of the derivatives according to the present invention for preparing medicaments for treating diseases.

### DESCRIPTION OF THE INVENTION

It is the object of the invention to provide derivatives of human amylin or analogues thereof which bind to albumin and/or the amylin receptor.

In one aspect of the invention a derivative of human amylin or an analogue thereof is provided, wherein
a) the amino acid residue in position 1 is any natural amino acid which is connected to a N-terminal extension consisting of 1-10 amino acids, wherein
   a. said extension is further linked to an albumin binding residue optionally via a linker, or
   b. an amino acid residue in position 2-37 has been substituted with a lysine residue, and said lysine residue is linked to an albumin binding residue, optionally via a linker;
   or
b) an amino acid residue in position 3, 17, 21, 25 or 29 has been substituted with a lysine residue and wherein said lysine residue is linked to an albumin binding residue, optionally via a linker;
   or
c) the amino acid residue in position 18 is arginine, and an amino acid residue in position 1-17 or 19-37 has been substituted with a lysine residue, and wherein said lysine residue is linked to an albumin binding residue, optionally via a linker;
   wherein the amino acid numbering conforms with the amino acid numbering in SEQ ID NO:17.

In one aspect of the invention a derivative of human amylin or an analogue thereof is provided, wherein
a) the amino acid residue in position 1 is any natural amino acid which is connected to a N-terminal extension consisting of 1-10 amino acids, wherein
   a. said extension is further linked to an albumin binding residue optionally via a linker, or
   b. an amino acid residue in position 2-37 has been substituted with a lysine residue, and said lysine residue is linked to an albumin binding residue, optionally via a linker;
   or
b) the amino acid residue in position 3 has been substituted with a lysine residue and wherein said lysine residue is linked to an albumin binding residue, optionally via a linker;
   or
c) an amino acid residue in position 17, 21, 25 or 29 has been substituted with a lysine residue and wherein said lysine residue is linked to an albumin binding residue, optionally via a linker;
   or
d) the amino acid residue in position 18 is arginine, and an amino acid residue in position 1-17 or 19-37 has been substituted with a lysine residue, and wherein said lysine residue is linked to an albumin binding residue, optionally via a linker;
   wherein the amino acid numbering conforms with the amino acid numbering in SEQ ID NO:17.

In one aspect of the invention a derivative of human amylin or an analogue thereof is provided, wherein
the amino acid residue in position 1 is any natural amino acid which is connected to a N-terminal extension consisting of 1-10 amino acids, wherein
   a. said extension is further linked to an albumin binding residue optionally via a linker, or
   b. an amino acid residue in position 2-37 has been substituted with a lysine residue, and said lysine residue is linked to an albumin binding residue, optionally via a linker;
and the amino acid residues in position 25, position 28 and position 29 has been substituted with proline and optionally the amino acid residue in position 18 is arginine;
   wherein the amino acid numbering conforms with the amino acid numbering in SEQ ID NO:17.

In one aspect of the invention a derivative of human amylin or an analogue thereof is provided, wherein
the amino acid residue in position 3, 17, 21, 25 or 29 has been substituted with a lysine residue and wherein said lysine residue is linked to an albumin binding residue, optionally via a linker and the amino acid residues in position 25, position 28 and position 29 has been substituted with proline and optionally the amino acid residue in position 18 is arginine;
   wherein the amino acid numbering conforms with the amino acid numbering in SEQ ID NO:17.

In one aspect of the invention a derivative of human amylin or an analogue thereof is provided, wherein
the amino acid residue in position 1 is glutamic acid or arginine, and wherein said glutamic acid or arginine is connected to a N-terminal extension consisting of 1-10 amino acids, wherein said extension is further linked to an albumin binding residue, optionally via a linker;
   wherein the amino acid numbering conforms with the amino acid numbering in SEQ ID NO:17.

In one aspect of the invention a derivative of human amylin or an analogue thereof is provided, wherein
the amino acid residue in position 1 is glutamic acid or arginine, and wherein said glutamic acid or arginine is connected to a N-terminal extension consisting of 1-6 amino acids, wherein said extension is further linked to an albumin binding residue optionally via a linker;
   wherein the amino acid numbering conforms with the amino acid numbering in SEQ ID NO:17.

In one aspect of the invention a derivative of human amylin or an analogue thereof is provided, wherein
the amino acid residue in position 1 is glutamic acid or arginine, and wherein said glutamic acid or arginine is connected to a N-terminal extension consisting of 5 amino acids, 4 amino acids, 3 amino acids, 2 amino acids or 1 amino acid, wherein said extension is further linked to an albumin binding residue optionally via a linker;
   wherein the amino acid numbering conforms with the amino acid numbering in SEQ ID NO:17.

In one aspect of the invention a derivative of human amylin or an analogue thereof is provided, wherein
the amino acid residue in position 1 is any natural amino acid which is connected to a N-terminal extension consisting of 1-10 amino acids, and an amino acid residue in position 2-37 has been substituted with a lysine residue, wherein said lysine residue is linked to an albumin binding residue, optionally via a linker;
   wherein the amino acid numbering conforms with the amino acid numbering in SEQ ID NO:17.

In one aspect a derivative of human amylin or an analogue thereof is provided, wherein
the amino acid residue in position 3 has been substituted with a lysine residue and wherein said lysine residue is linked to an albumin binding residue, optionally via a linker;
   wherein the amino acid numbering conforms with the amino acid numbering in SEQ ID NO:17.

In one aspect a derivative of human amylin or an analogue thereof is provided, wherein
an amino acid residue in position 17, 21, 25 or 29 has been substituted with a lysine residue and wherein said lysine residue is linked to an albumin binding residue, optionally via a linker;
   wherein the amino acid numbering conforms with the amino acid numbering in SEQ ID NO:17.

In one aspect of the invention a derivative of human amylin or an analogue thereof is provided, wherein
the amino acid residue in position 17 has been substituted with a lysine residue and wherein said lysine residue is linked to an albumin binding residue, optionally via a linker;
   wherein the amino acid numbering conforms with the amino acid numbering in SEQ ID NO:17.

In one aspect of the invention a derivative of human amylin or an analogue thereof is provided, wherein
the amino acid residue in position 21 has been substituted with a lysine residue and wherein said lysine residue is linked to an albumin binding residue, optionally via a linker;
   wherein the amino acid numbering conforms with the amino acid numbering in SEQ ID NO:17.

In one aspect of the invention a derivative of human amylin or an analogue thereof is provided, wherein
the amino acid residue in position 29 has been substituted with a lysine residue and wherein said lysine residue aspect is linked to an albumin binding residue, optionally via a linker;
   wherein the amino acid numbering conforms with the amino acid numbering in SEQ ID NO:17.

In one aspect a derivative of human amylin or an analogue thereof is provided, wherein
the amino acid residue in position 18 is arginine, and an amino acid residue in position 1-17 or 19-37 has been substituted with a lysine residue, and wherein said lysine residue is linked to an albumin binding residue, optionally via a linker;
   wherein the amino acid numbering conforms with the amino acid numbering in SEQ ID NO:17.

In one aspect a derivative of human amylin or an analogue thereof is provided, wherein
the amino acid residue in position 1 is glutamic acid or arginine, the amino acid residues in positions 25, 28 and 29 are proline, and wherein said glutamic acid or arginine is connected to a N-terminal extension consisting of 5 amino acids, 4 amino acids, 3 amino acids, 2 amino acids or 1 amino acid, wherein said extension is further linked to an albumin binding residue optionally via a linker;
   wherein the amino acid numbering conforms with the amino acid numbering in SEQ ID NO:17.

In one aspect a derivative of human amylin or an analogue thereof is provided, wherein
the amino acid residues in positions 25, 28 and 29 are proline, and an amino acid residue in position 1-17 or 19-37 has been substituted with a lysine residue, and wherein said lysine residue is linked to an albumin binding residue, optionally via a linker;
   wherein the amino acid numbering conforms with the amino acid numbering in SEQ ID NO:17.

In one aspect a derivative of human amylin or an analogue thereof is provided, wherein
the amino acid residue in position 1 is glutamic acid or arginine, the amino acid residue in position 18 is arginine and the amino acid residues in positions 25, 28 and 29 are proline, and wherein said glutamic acid or arginine is connected to a N-terminal extension consisting of 5 amino acids, 4 amino acids, 3 amino acids, 2 amino acids or 1 amino acid, wherein said extension is further linked to an albumin binding residue optionally via a linker;
   wherein the amino acid numbering conforms with the amino acid numbering in SEQ ID NO:17.

In one aspect a derivative of human amylin or an analogue thereof is provided, wherein
the amino acid residue in position 18 is arginine, the amino acid residues in positions 25, 28 and 29 are proline, and an amino acid residue in position 1-17 or 19-37 has been substituted with a lysine residue, and wherein said lysine residue is linked to an albumin binding residue, optionally via a linker;
   wherein the amino acid numbering conforms with the amino acid numbering in SEQ ID NO:17.

The following is a non-limiting list of aspects, which is further described elsewhere herein:
1. A derivative of human amylin with SEQ ID NO:17 or an analogue thereof, wherein
   a) the amino acid residue in position 1 is any natural amino acid which is connected to a N-terminal extension consisting of 1-10 amino acids, wherein
      a. said extension is further linked to an albumin binding residue, optionally via a linker, or
      b. an amino acid residue in position 2-37 has been substituted with a lysine residue, and said lysine residue is linked to an albumin binding residue, optionally via a linker;
      or
   b) the amino acid residue in position 3, 17 21, 25 or 29 has been substituted with a lysine residue and wherein said lysine residue is linked to an albumin binding residue, optionally via a linker;
      or
   c) the amino acid residue in position 18 is arginine, and an amino acid residue in position 1-17 or 19-37 has been substituted with a lysine residue, and wherein said lysine residue is linked to an albumin binding residue, optionally via a linker;
      wherein the amino acid numbering conforms with the amino acid numbering in SEQ ID NO:17.
2. A derivative of human amylin with SEQ ID NO:17 or an analogue thereof according to aspect 1, wherein
   a) the amino acid residue in position 1 is any natural amino acid which is connected to a N-terminal extension consisting of 1-10 amino acids, wherein
      a. said extension is further linked to an albumin binding residue, optionally via a linker, or
      b. an amino acid residue in position 2-37 has been substituted with a lysine residue, and said lysine residue is linked to an albumin binding residue, optionally via a linker;
      or
   b) the amino acid residue in position 3 has been substituted with a lysine residue and wherein said lysine residue is linked to an albumin binding residue, optionally via a linker;
      or
   c) an amino acid residue in position 17, 21, 25 or 29 has been substituted with a lysine residue and wherein said lysine residue is linked to an albumin binding residue, optionally via a linker;
      or
   d) the amino acid residue in position 18 is arginine, and an amino acid residue in position 1-17 or 19-37 has been substituted with a lysine residue, and wherein said lysine residue is linked to an albumin binding residue, optionally via a linker;
   wherein the amino acid numbering conforms with the amino acid numbering in SEQ ID NO:17.
3. A derivative of human amylin with SEQ ID NO:17 or an analogue thereof according to aspect 1 or 2, wherein
   the amino acid residue in position 1 is any natural amino acid which is connected to a N-terminal extension consisting of 1-10 amino acids, wherein
      a) said extension is further linked to an albumin binding residue, optionally via a linker
      b) an amino acid residue in position 2-37 has been substituted with a lysine residue, and said lysine residue is linked to an albumin binding residue, optionally via a linker;
   and the amino acid residues in position 25, position 28 and position 29 has been substituted with proline and optionally the amino acid residue in position 18 is arginine;
   wherein the amino acid numbering conforms with the amino acid numbering in SEQ ID NO:17.
4. A derivative of human amylin with SEQ ID NO:17 or an analogue thereof according to aspect 1 or 2, wherein
   the amino acid residue in position 3, 17, 21, 25 or 29 has been substituted with a lysine residue and wherein said lysine residue is linked to an albumin binding residue, optionally via a linker and the amino acid residues in position 25, position 28 and position 29 has been substituted with proline and optionally the amino acid residue in position 18 is arginine;
   wherein the amino acid numbering conforms with the amino acid numbering in SEQ ID NO:17.
5. The derivative according to any one of aspects 1-4, wherein the derivative is human amylin of SEQ ID No 17 or an analogue thereof, wherein
   an amino acid residue in position 3 has been substituted with a lysine residue and wherein such a lysine residue is linked to an albumin binding residue, optionally via a linker.
6. The derivative according to aspect 6, wherein the derivative is human amylin of SEQ ID NO:17 or an analogue thereof, wherein
   an amino acid residue in position 17 has been substituted with a lysine residue and wherein such a lysine residue is linked to an albumin binding residue, optionally via a linker.
7. The derivative according to any one of aspects 1-4, wherein the derivative is human amylin of SEQ ID NO:17 or an analogue thereof, wherein
   an amino acid residue in position 21 has been substituted with a lysine residue and wherein such a lysine residue is linked to an albumin binding residue, optionally via a linker.
8. The derivative according to any one of aspects 1-4, wherein the derivative is human amylin of SEQ ID NO:17 or an analogue thereof, wherein
   an amino acid residue in position 25 has been substituted with a lysine residue and wherein such a lysine residue is linked to an albumin binding residue, optionally via a linker.
9. The derivative according to any one of aspects 1-4, wherein the derivative is human amylin of SEQ ID NO:17 or an analogue thereof, wherein
   an amino acid residue in position 29 has been substituted with a lysine residue and wherein such a lysine residue is linked to an albumin binding residue, optionally via a linker.
10. The derivative according to any one of aspects 1-4, wherein the derivative is human amylin of SEQ ID NO:17 or an analogue thereof, wherein
   the amino acid in position 18 is arginine, the amino acids in position 25, position 28 and position 29 are proline, and an amino acid residue in position 1-17 or 19-37 has been substituted with a lysine residue, and wherein such a lysine residue is linked to an albumin binding residue, optionally via a linker.
11. The derivative according to any one of aspects 1-10, wherein the derivative has from 1-12 amino acid substitutions compared to human amylin.
12. The derivative according to any one of aspects 1-11, wherein the derivative has from 3-10 amino acid substitutions compared to human amylin.
13. The derivative according to any one of aspects 1-12, wherein the derivative has 3, 4, 5, 6, 7, 8 or 9 amino acid substitutions compared to human amylin.
14. The derivative according to any one of aspects 1-13, wherein the derivative is an analogue of human amylin of SEQ ID NO:17, wherein 0-8 additional charges have been added compared to human amylin.
15. The derivative according to aspect 14, wherein the 0-8 additional charges have been added by substituting one or more amino acid residues of human amylin or analogue thereof with charged amino acids and/or by adding charged amino acids in a N-terminal extension, or by adding negatively charged entities in the albumin binding residue or polyethylene glycol polymer and/or the linker.
16. The derivative according to aspect 14 or 15, wherein the 0-8 additional charges have been added by substituting one or more amino acid residues of human amylin or an analogue thereof with glutamic acid residue(s) and/or aspartic acid residue(s) and/or by adding charged amino acids in a N-terminal extension.
17. The derivative according any of the aspects 14-16, wherein the 0-8 additional charges have been added by adding charged amino acids in a N-terminal extension.
18. The derivative according to any of the aspects 1-17, wherein the derivative comprises an N-terminal extension consisting of 1-10 amino acids, wherein said extension is further linked to an albumin binding residue, optionally via a linker.
19. The derivative according to aspect 18, wherein the derivative comprises an N-terminal extension consisting of 1-8 amino acids, wherein said extension is further linked to an albumin binding residue. optionally via a linker
20. The derivative according to aspect 19, wherein the derivative comprises an N-terminal extension consisting of 1-6 amino acids, wherein said extension is further linked to an albumin binding residue. optionally via a linker
21. The derivative according to aspect 20, wherein the derivative comprises an N-terminal extension consisting of 5 amino acids, wherein said extension is further linked to an albumin binding residue. optionally via a linker
22. The derivative according to aspect 20, wherein the derivative comprises an N-terminal extension consisting of 4 amino acids, wherein said extension is further linked to an albumin binding residue optionally via a linker.
23. The derivative according to aspect 20, wherein the derivative comprises an N-terminal extension consisting of 3 amino acids, wherein said extension is further linked to an albumin binding residue optionally via a linker.
24. The derivative according to aspect 23, wherein the N-terminal extension are selected from the group consisting of Glu-Glu-Arg, Glu-Glu-Glu and Glu-Lys-Arg.
25. The derivative according to aspect 20, wherein the derivative comprises an N-terminal extension consisting of 2 amino acids, wherein said extension is further linked to an albumin binding residue optionally via a linker.
26. The derivative according to aspect 25, wherein the N-terminal extension is Glu-Lys
27. The derivative according to aspect 20, wherein the derivative comprises an N-terminal extension consisting of 1 amino acid, wherein said extension is further linked to an albumin binding residue optionally via a linker.
28. The derivative according to aspect 25, wherein the N-terminal extension are selected from the group consisting of Arg, Lys and Glu.
29. A derivative according to aspect 1 comprising an amino acid sequence of formula 1:

(SEQ ID No: 18)

Formula (1)
Xaa₁-Cys-Xaa₃-Thr-Ala-Thr-Cys-Ala-Thr-Gln-Arg-Leu-
Ala-Asn-Phe-Leu-Xaa₁₇-Xaa₁₈-Ser-Ser-Xaa₂₁-Asn-
Xaa₂₃-Gly-Xaa₂₅-Xaa₂₆-Leu-Xaa₂₈-Xaa₂₉-Thr-Asn-Val-
Gly-Ser-Asn-Thr-Tyr

wherein
Xaa₁ is deleted or independently selected from Lys, Arg and Glu;
Xaa₃ is independently selected from Asn, Gly, Gln and Lys;
Xaa₁₇ is independently selected from Ala, Val and Lys;
Xaa₁₈ is independently selected from His, Pro and Arg;
Xaa₂₁ is independently selected from Asn, Gln and Lys;
Xaa₂₃ is independently selected from Phe and Leu;
Xaa₂₅ is independently selected from Ala, Pro and Lys;
Xaa₂₆ is independently selected from Val and Ile;
Xaa₂₈ is independently selected from Ser and Pro;
Xaa₂₉ is independently selected from Ser, Pro and Lys;
the C-terminal may optionally be derivatized as an amide;
the N-terminal may optionally be extended with 1-10 amino acid residues
wherein the amino acid residue Xaa₁ is connected to a N-terminal extension consisting of 1-10 amino acids, which extension can be further linked to an albumin binding residue, optionally via a linker, or
the amino acid residue Xaa₁, Xaa₃, Xaa₁₇, Xaa₂₁, Xaa₂₅ or Xaa₂₉ is lysine, and said lysine residue is linked to an albumin binding residue, optionally via a linker;
wherein if Xaa₁ is Lys and Xaa₃ is Asn and Xaa₂₁ is Asn and Xaa₂₅ is Ala or Pro, then Xaa₂₉ is Lys; and
   if Xaa₁ is Glu or Arg, then said Glu or Arg is connected to a N-terminal extension consisting of 1-10 amino acids, and said extension is further linked to an albumin binding residue, and
   if Xaa₁ is Lys, then one amino acid in a position selected from the group consisting of Xaa₁, Xaa₃, Xaa₂₁, Xaa₂₅ and Xaa₂₉ in formula (I) is linked to an albumin binding residue, optionally via a linker.
30. The derivative according to aspect 29, wherein the albumin binding residue is a lipophilic residue.
31. The derivative according to aspect 29, wherein the albumin binding residue is negatively charged at physiological pH.
32. The derivative according to aspect 29, wherein the albumin binding residue comprises a group which can be negatively charged at physiological pH.
33. The derivative according to aspect 32, wherein the albumin binding residue comprises a carboxylic acid group.
34. The derivative according to any one of the aspects 29-33, wherein the albumin binding residue binds non-covalently to albumin.
35. The derivative according to any one of the aspects 29-34, wherein the albumin binding residue has a binding affinity towards human serum albumin that is below about 10 μM or below about 1 μM.
36. The derivative according to aspect 29, wherein the albumin binding residue is selected from the group consisting of a straight chain alkyl group, a branched alkyl group, a group which has an ω-carboxylic acid group, and a partially or completely hydrogenated cyclopentanophenanthrene skeleton.
37. The derivative according to aspect 29, wherein the albumin binding residue is a cibacronyl residue.
38. The derivative according to aspect 29, wherein the lipophilic residue comprises a partially or completely hydrogenated cyclopentanophenathrene skeleton.
39. The derivative according to aspects 29-30, wherein the albumin binding residue has from 6 to 40 carbon atoms, from 8 to 26 carbon atoms or from 8 to 20 carbon atoms.
40. The derivative according to aspects 29-30, wherein the albumin binding residue is an acyl group selected from the group comprising CH₃(CH₂)ᵣCO—, wherein r is an integer from 4 to 38, preferably an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.
41. The derivative according to aspects 29-30, wherein the albumin binding residue is an acyl group of a straight-chain or branched alkane α,ω-dicarboxylic acid.
42. The derivative according to aspects 29-30, wherein the albumin binding residue is an acyl group selected from the group comprising HOOC(CH₂)ₛCO—, wherein s is an integer from 4 to 38, preferably an integer from 4 to 24, more preferred selected from the group comprising HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.
43. The derivative according to aspects 29-30, wherein the albumin binding residue is a group of the formula CH₃(CH₂)ᵥCO—NHCH(COOH)(CH₂)₂CO—, wherein v is an integer from 10 to 24.
44. The derivative according to aspects 29-30, wherein the albumin binding residue is a group of the formula CH₃(CH₂)_{w}CO—NHCH((CH₂)₂COOH)CO—, wherein w is an integer from 8 to 24.
45. The derivative according to aspects 29-30, wherein the albumin binding residue is a group of the formula —NHCH(COOH)(CH₂)₄NH—CO(CH₂)_{y}CH₃, wherein y is an integer from 8 to 18.
46. The derivative according to aspect 29, wherein the albumin binding residue is

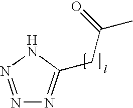

where l is 12, 13, 14, 15, 16, 17, 18, 19 or 20.
47. The derivative according to any one of the aspects 29-46, wherein the albumin binding residue via a linker is connected via the ε-amino group of a lysine residue.
48. The derivative according to any of the aspects 29-47, wherein the linker comprises 1-4 amino acid residues.
49. The derivative according to aspect 48, wherein the linker is Glu-Glu-Glu-Glu, Glu-Arg-Glu-Glu, Glu, Glu-Glu, Glu-Glu-Arg.
50. The derivative according to aspects 29-49 wherein the linker comprises one or more alkylene glycol units, for example 1 to 5 ethylene glycol units.
51. The derivative according to any of aspects 29-50, wherein the linker comprises
   —C(O)—(CH₂)ₗ—O—[(CH₂CH₂—O]ₘ—(CH₂)ₚ—[NHC(O)—(CH₂)ₗ—O—[(CH₂)ₙ—O]ₘ—(CH₂)ₚ]_{q}—NH—, wherein l, m, n, and p independently are 1-5, and q is 0-5.
52. The derivative according to any of aspects 29-51, wherein the linker comprises a hexadecanoylsulfamoyl moiety.
53. A pharmaceutical composition comprising a derivative according to any one of the aspects 1-52, and a pharmaceutically acceptable excipient.
54. The pharmaceutical composition according to aspect 53, which is suited for parenteral administration.
55. A derivative according to any one of the aspects 1-52 for use as a medicament.
56. A derivative according to any one of the aspects 1-52 for use as a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers.
57. A derivative according to any one of the aspects 1-52 for use as a medicament for delaying or preventing disease progression in type 2 diabetes.
58. A derivative according to any one of the aspects 1-52 for use as a medicament for decreasing food intake, decreasing β-cell apoptosis, increasing β-cell function and 8-cell mass, and/or for restoring glucose sensitivity to β-cells. Use of a derivative according to any one of the aspects 1-42 for the preparation of a medicament.
59. Use of a derivative according to any one of the aspects 1-52 for the preparation of a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers.
60. Use of a derivative according to any one of the aspects 1-52 for the preparation of a medicament for delaying or preventing disease progression in type 2 diabetes.
61. Use of a derivative according to any one of the aspects 1-52 for the preparation of a medicament for decreasing food intake, decreasing β-cell apoptosis, increasing β-cell function and β-cell mass, and/or for restoring glucose sensitivity to β-cells.
62. Method for increasing the time of action in a patient of human amylin or an analog thereof, characterized by modifying
   a) the amino acid residue in position 1 by optionally substituting with any natural amino acid, and by connecting said natural amino acid to a N-terminal extension consisting of 1-10 amino acids, and further
      a. linking said extension to an albumin binding residue, optionally via a linker, or
      b. modifying an amino acid residue in position 2-37 by substitution with a lysine residue, and by linking said lysine residue to an albumin binding residue, optionally via a linker,
      or;
   b) the amino acid residue in position 3, 17, 21, 25 or 29 by substitution with a lysine residue and by linking said lysine residue to an albumin binding residue, optionally via a linker,
      or;
   c) the amino acid residue in position 18 by substitution with an arginine residue and an amino acid residue in position 2-17 or 19-37 by substitution with a lysine residue and by linking said lysine residue to an albumin binding residue, optionally via a linker;
   d) the amino acid in position 18 is arginine, the amino acids in position 25, position 28 and position 29 are proline, and an amino acid residue in position 1-17 or 19-37 has been substituted with a lysine residue, and wherein such a lysine residue is linked to an albumin binding residue, optionally via a linker.
      wherein the amino acid numbering conforms with the amino acid numbering in SEQ ID NO:17.
63. Method according to aspect 62, wherein the time of action in a patient of human amylin or an analog thereof is increased to more than about 40 hours.
64. Method according to any one of the aspects 62 or 63, wherein the human amylin or an analog thereof is as further defined in any one of the aspects 1-52.
65. A method for the treatment, prevention or alleviation of hyperglycemia, type 2 diabetes, impaired glucose tolerance, type 1 diabetes, obesity, syndrome X or dyslipidemia in a subject comprising administering to a subject a derivative according to any one of aspects 1-52 or a pharmaceutical composition according to aspects 53 or 54.
66. A process for preparing a pharmaceutical composition according to aspect 53 or 54 comprising mixing a derivative according to any one of aspects 1-52 with pharmaceutically acceptable substances and/or excipients.
67. Derivative of amylin according to the examples.

The invention is further summarized in the following aspects:

1a. A derivative of human amylin with SEQ ID NO:17 or an analogue thereof, wherein
the amino acid residue in position 1 is any natural amino acid which is connected to a N-terminal extension consisting of 1-10 amino acids, wherein
said extension is further linked to an albumin binding residue, or
an amino acid residue in position 2-37 has been substituted with a lysine residue, and said lysine residue is linked to an albumin binding residue, optionally via a linker;
or
the amino acid residue in position 3, 21, 25 or 29 has been substituted with a lysine residue and wherein said lysine residue is linked to an albumin binding residue, optionally via a linker;
or
the amino acid residue in position 18 is arginine, and an amino acid residue in position 1-17 or 19-37 has been substituted with a lysine residue, and wherein said lysine residue is linked to an albumin binding residue or a polyethylene glycol polymer, optionally via a linker;

wherein the amino acid numbering conforms with the amino acid numbering in SEQ ID NO:17.

2a. A derivative of human amylin with SEQ ID NO:17 or an analogue thereof according to aspect 1a, wherein
the amino acid residue in position 1 is any natural amino acid which is connected to a N-terminal extension consisting of 1-10 amino acids, wherein
said extension is further linked to an albumin binding residue or a polyethylene glycol polymer,
or
an amino acid residue in position 2-37 has been substituted with a lysine residue, and said lysine residue is linked to an albumin binding residue or a polyethylene glycol polymer, optionally via a linker;
or
the amino acid residue in position 3 has been substituted with a lysine residue and wherein said lysine residue is linked to an albumin binding residue or a polyethylene glycol polymer, optionally via a linker;
or
an amino acid residue in position 21, 25 or 29 has been substituted with a lysine residue and wherein said lysine residue is linked to an albumin binding residue or a polyethylene glycol polymer, optionally via a linker;
or
the amino acid residue in position 18 is arginine, and an amino acid residue in position 1-17 or 19-37 has been substituted with a lysine residue, and wherein said lysine residue is linked to an albumin binding residue or a polyethylene glycol polymer, optionally via a linker;
wherein the amino acid numbering conforms with the amino acid numbering in SEQ ID NO:17.
3a. A derivative of human amylin with SEQ ID NO:17 or an analogue thereof according to aspect 1a or 2a, wherein
the amino acid residue in position 1 is any natural amino acid which is connected to a N-terminal extension consisting of 1-10 amino acids, wherein
said extension is further linked to an albumin binding residue or a polyethylene glycol polymer, or
an amino acid residue in position 2-37 has been substituted with a lysine residue, and said lysine residue is linked to an albumin binding residue or a polyethylene glycol polymer, optionally via a linker;
and optionally the amino acid residue in position 18 is arginine;
wherein the amino acid numbering conforms with the amino acid numbering in SEQ ID NO:17.
4a. A derivative of human amylin with SEQ ID NO:17 or an analogue thereof according to aspect 1a or 2a, wherein
the amino acid residue in position 3, 21, 25 or 29 has been substituted with a lysine residue and wherein said lysine residue is linked to an albumin binding residue or a polyethylene glycol polymer, optionally via a linker;
and optionally the amino acid residue in position 18 is arginine;
wherein the amino acid numbering conforms with the amino acid numbering in SEQ ID NO:17.
5a. The derivative according to aspect 1a or 2a, wherein the derivative is human amylin of SEQ ID NO:17, wherein
the amino acid residue in position 1 is any natural amino acid which is connected to a N-terminal extension consisting of 1-10 amino acids, wherein
said extension is further linked to an albumin binding residue or a polyethylene glycol polymer,
or
an amino acid residue in position 2-37 has been substituted with a lysine residue, and said lysine residue is linked to an albumin binding residue or a polyethylene glycol polymer, optionally via a linker;
or
an amino acid residue in position 3, 21, 25 or 29 has been substituted with a lysine residue and wherein said lysine residue is linked to an albumin binding residue or a polyethylene glycol polymer, optionally via a linker;
or
the amino acid residue in position 18 is arginine, and an amino acid residue in position 1-17 or 19-37 has been substituted with a lysine residue, and wherein said lysine residue is linked to an albumin binding residue or a polyethylene glycol polymer, optionally via a linker.
6a. The derivative according to any one of aspects 1a-4-a, wherein the derivative is human amylin of SEQ ID No 17 or an analogue thereof, wherein
an amino acid residue in position 3 has been substituted with a lysine residue and wherein such a lysine residue is linked to an albumin binding residue or a polyethylene glycol polymer, optionally via a linker.
7a. The derivative according to aspect 6a, wherein the derivative is human amylin of SEQ ID NO:17 or an analogue thereof, wherein
an amino acid residue in position 3 has been substituted with a lysine residue and wherein such a lysine residue is linked to an albumin binding residue, optionally via a linker
8a. The derivative according to any one of aspects 1a-4-a, wherein the derivative is human amylin of SEQ ID NO:17 or an analogue thereof, wherein
an amino acid residue in position 21 has been substituted with a lysine residue and wherein such a lysine residue is linked to an albumin binding residue or a polyethylene glycol polymer, optionally via a linker.
9a. The derivative according to any one of aspects 1a-4-a, wherein the derivative is human amylin of SEQ ID NO:17 or an analogue thereof, wherein
an amino acid residue in position 25 has been substituted with a lysine residue and wherein such a lysine residue is linked to an albumin binding residue or a polyethylene glycol polymer, optionally via a linker.
10a. The derivative according to any one of aspects 1a-4-a, wherein the derivative is human amylin of SEQ ID NO:17 or an analogue thereof, wherein
an amino acid residue in position 29 has been substituted with a lysine residue and wherein such a lysine residue is linked to an albumin binding residue or a polyethylene glycol polymer, optionally via a linker.
11a. The derivative according to any one of aspects 1a-4-a, wherein the derivative is human amylin of SEQ ID NO:17 or an analogue thereof, wherein
the amino acid in position 18 is arginine, and an amino acid residue in position 1-17 or 19-37 has been substituted with a lysine residue, and wherein such a lysine residue is linked to an albumin binding residue or a polyethylene glycol polymer, optionally via a linker.
12a. The derivative according to any one of aspects 1a-11a, wherein the derivative has from 1-12 amino acid substitutions compared to human amylin.
13a. The derivative according to any one of aspects 1a-12a, wherein the derivative has from 1-10 amino acid substitutions compared to human amylin.
14a. The derivative according to any one of aspects 1a-13a, wherein the derivative has from 1-8 amino acid substitutions compared to human amylin.
15a. The derivative according to any one of aspects 1-14, wherein the derivative has from 1-6 amino acid substitutions compared to human amylin.
16a. The derivative according to any one of aspects 1a-15a, wherein the derivative has from 1-4 amino acid substitutions compared to human amylin.
17a. The derivative according to any one of aspects 1a-16a, wherein the derivative is an analogue of human amylin of SEQ ID NO:17, wherein 0-8 additional charges have been added compared to human amylin.
18a. The derivative according to aspect 17a, wherein the 0-8 additional charges have been added by substituting one or more amino acid residues of human amylin or analogue thereof with charged amino acids and/or by adding charged amino acids in a N-terminal extension, or by adding negatively charged entities in the albumin binding residue or polyethylene glycol polymer and/or the linker.
19a. The derivative according to aspects 17a or 18a, wherein the 0-8 additional charges have been added by substituting one or more amino acid residues of human amylin or an analogue thereof with glutamic acid residue(s) and/or aspartic acid residue(s) and/or by adding charged amino acids in a N-terminal extension.
20a. The derivative according any of the aspects 17a-19a, wherein the 0-8 additional charges have been added by adding charged amino acids in a N-terminal extension.
21a. The derivative according to any of the aspects 1a-3a, 5a or 12a-20a, wherein the derivative comprises an N-terminal extension consisting of 1-10 amino acids, wherein said extension is further linked to an albumin binding residue or a polyethylene glycol polymer.
22a. The derivative according to aspect 21a, wherein the derivative comprises an N-terminal extension consisting of 1-8 amino acids, wherein said extension is further linked to an albumin binding residue or a polyethylene glycol polymer.
23a. The derivative according to aspect 22a, wherein the derivative comprises an N-terminal extension consisting of 1-6 amino acids, wherein said extension is further linked to an albumin binding residue or a polyethylene glycol polymer.
24a. The derivative according to aspect 23a, wherein the derivative comprises an N-terminal extension consisting of 5 amino acids, wherein said extension is further linked to an albumin binding residue or a polyethylene glycol polymer.
25a. The derivative according to aspect 24a, wherein the derivative comprises an N-terminal extension consisting of 4 amino acids, wherein said extension is further linked to an albumin binding residue or a polyethylene glycol polymer.
26a. The derivative according to aspect 25a, wherein the derivative comprises an N-terminal extension consisting of 3 amino acids, wherein said extension is further linked to an albumin binding residue or a polyethylene glycol polymer.
27a. The derivative according to aspect 26a, wherein the derivative comprises an N-terminal extension consisting of 2 amino acids, wherein said extension is further linked to an albumin binding residue or a polyethylene glycol polymer.
28a. The derivative according to aspect 27a, wherein the derivative comprises an N-terminal extension consisting of 1 amino acid, wherein said extension is further linked to an albumin binding residue or a polyethylene glycol polymer.
29a. A derivative according to aspect 1a comprising an amino acid sequence of formula 1:

(SEQ ID No: 18)

Formula (1)
Xaa₁-Cys-Xaa₃-Thr-Ala-Thr-Cys-Ala-Thr-Gln-Arg-Leu-
Ala-Asn-Phe-Leu-Xaa₁₇-Xaa₁₈-Ser-Ser-Xaa₂₁-Asn-
Xaa₂₃-Gly-Xaa₂₅-Xaa₂₆-Leu-Xaa₂₈-Xaa₂₉-Thr-Asn-Val-
Gly-Ser-Asn-Thr-Tyr

wherein
Xaa₁ is deleted or independently selected from Lys and Glu;
Xaa₃ is independently selected from Asn, Gly and Lys;
Xaa₁₇ is independently selected from Ala, Val and Lys;
Xaa₁₆ is independently selected from His, Pro and Arg;
Xaa₂₁ is independently selected from Asn, Gln and Lys;
Xaa₂₃ is independently selected from Phe and Leu;
Xaa₂₅ is independently selected from Ala, Pro and Lys;
Xaa₂₆ is independently selected from Val and Ile;
Xaa₂₆ is independently selected from Ser and Pro;
Xaa₂₉ is independently selected from Ser, Pro and Lys;
the C-terminal may optionally be derivatized as an amide;
the N-terminal may optionally be extended with 1-10 amino acid residues
wherein if Xaa₁ is Lys and Xaa₃ is Asn and Xaa₂₁ is Asn and Xaa₂₅ is Ala or Pro, then Xaa₂₉ is Lys; and
if Xaa₁ is Glu, then said Glu is connected to a N-terminal extension consisting of 1-10 amino acids, and said extension is further linked to an albumin binding residue or a polyethylene glycol polymer, and
if Xaa₁ is Lys, then one amino acid in a position selected from the group consisting of Xaa₁, Xaa₃, Xaa₂₁, Xaa₂₅ and Xaa₂₉ in formula (I) is linked to an albumin binding residue or a polyethylene glycol polymer, optionally via a linker.
30a. The derivative according to any one of the aspects 1a-29a, wherein the derivative is linked to a polyethylene glycol polymer.
31a. The derivative according to aspect 30a, wherein the poly ethylene glycol polymer is a polyethylene glycol having a molecular weight of at least 30 kD.
32a. The derivative according to any one of the aspects 30a-31a, wherein the polyethylene glycol polymer is a polyethylene glycol which is branched.
33a. The derivative according to any one of the aspects 1a-29a, wherein the derivative is linked to an albumin binding residue.
34a. The derivative according to aspect 33a, wherein the albumin binding residue is a lipophilic residue.
35a. The derivative according to aspect 33a, wherein the albumin binding residue is negatively charged at physiological pH.
36a. The derivative according to aspect 33a, wherein the albumin binding residue comprises a group which can be negatively charged.
37a. The derivative according to aspect 36a, wherein the albumin binding residue comprises a carboxylic acid group.
38a. The derivative according to any one of the aspects 33a-37a, wherein the albumin binding residue binds non-covalently to albumin.
39a. The derivative according to any one of the aspects 33a-38a, wherein the albumin binding residue has a binding affinity towards human serum albumin that is below about 10 μM or below about 1 μM.
40a. The derivative according to aspect 33a, wherein the albumin binding residue is selected from the group consisting of a straight chain alkyl group, a branched alkyl group, a group which has an ω-carboxylic acid group, and a partially or completely hydrogenated cyclopentanophenanthrene skeleton.
41a. The derivative according to aspect 33a, wherein the albumin binding residue is a cibacronyl residue.
42a. The derivative according to aspect 34a, wherein the lipophilic residue comprises a partially or completely hydrogenated cyclopentanophenathrene skeleton.
43a. The derivative according to aspect 34a, wherein the albumin binding residue has from 6 to 40 carbon atoms, from 8 to 26 carbon atoms or from 8 to 20 carbon atoms.
44a. The derivative according to aspect 34a, wherein the albumin binding residue is an acyl group selected from the group comprising CH₃(CH₂)ᵣCO—, wherein r is an integer from 4 to 38, preferably an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.
45a. The derivative according to aspect 34a, wherein the albumin binding residue is an acyl group of a straight-chain or branched alkane α,ω-dicarboxylic acid.
46a. The derivative according to aspect 34a, wherein the albumin binding residue is an acyl group selected from the group comprising HOOC(CH₂)ₛCO—, wherein s is an integer from 4 to 38, preferably an integer from 4 to 24, more preferred selected from the group comprising HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.
47a. The derivative according to aspect 34a, wherein the albumin binding residue is a group of the formula CH₃(CH₂)ᵥCO—NHCH(COOH)(CH₂)₂CO—, wherein v is an integer from 10 to 24.
48a. The derivative according to aspect 34a, wherein the albumin binding residue is a group of the formula CH₃(CH₂)_{w}CO—NHCH((CH₂)₂COOH)CO—, wherein w is an integer from 8 to 24.
49a. The derivative according to aspect 34a, wherein the albumin binding residue is a group of the formula COOH(CH₂)ₓCO—, wherein x is an integer from 8 to 24.
50a. The derivative according to aspect 34a, wherein the albumin binding residue is a group of the formula —NHCH(COOH)(CH₂)₄NH—CO(CH₂)_{y}CH₃, wherein y is an integer from 8 to 18.
51a. The derivative according to aspect 33a, wherein the albumin binding residue is a peptide, such as a peptide comprising less than 40 amino acid residues.
52a. The derivative according to any one of the aspects 33a-51a, wherein the albumin binding residue optionally via a linker is connected via the ε-amino group of a lysine residue.
53a. A pharmaceutical composition comprising a derivative according to any one of the aspects 1a-52a, and a pharmaceutically acceptable excipient.
54a. The pharmaceutical composition according to aspect 53a, which is suited for parenteral administration.
55a. A derivative according to any one of the aspects 1a-52a for use as a medicament.
56a. A derivative according to any one of the aspects 1a-52a for use as a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers.
57a. A derivative according to any one of the aspects 1a-52a for use as a medicament for delaying or preventing disease progression in type 2 diabetes. 58a. A derivative according to any one of the aspects 1a-52a for use as a medicament for decreasing food intake, decreasing β-cell apoptosis, increasing β-cell function and β-cell mass, and/or for restoring glucose sensitivity to β-cells.
59a. Use of a derivative according to any one of the aspects 1a-42a for the preparation of a medicament.
60a. Use of a derivative according to any one of the aspects 1a-52a for the preparation of a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers.
61a. Use of a derivative according to any one of the aspects 1a-52a for the preparation of a medicament for delaying or preventing disease progression in type 2 diabetes.
62a. Use of a derivative according to any one of the aspects 1a-52a for the preparation of a medicament for decreasing food intake, decreasing β-cell apoptosis, increasing β-cell function and β-cell mass, and/or for restoring glucose sensitivity to β-cells.
63a. Method for increasing the time of action in a patient of human amylin or an analog thereof, characterized by modifying
the amino acid residue in position 1 by optionally substituting with any natural amino acid, and by connecting said natural amino acid to a N-terminal extension consisting of 1-10 amino acids, and further
linking said extension to an albumin binding residue or a polyethylene glycol polymer, or
modifying an amino acid residue in position 2-37 by substitution with a lysine residue, and by linking said lysine residue to an albumin binding residue or a polyethylene glycol polymer, optionally via a linker,
or;
the amino acid residue in position 3, 21, 25 or 29 by substitution with a lysine residue and by linking said lysine residue to an albumin binding residue or a polyethylene glycol polymer, optionally via a linker,
or;
the amino acid residue in position 18 by substitution with an arginine residue and an amino acid residue in position 2-17 or 19-37 by substitution with a lysine residue and by linking said lysine residue to an albumin binding residue or a polyethylene glycol polymer, optionally via a linker;
wherein the amino acid numbering conforms with the amino acid numbering in SEQ ID NO:17.
64a. Method according to aspect 63a, wherein the time of action in a patient of human amylin or an analog thereof is increased to more than about 40 hours.
65a. Method according to any one of the aspects 63a or 64a, wherein the human amylin or an analog thereof is as further defined in any one of the aspects 1a-52a.
66a. A method for the treatment, prevention or alleviation of hyperglycemia, type 2 diabetes, impaired glucose tolerance, type 1 diabetes, obesity, syndrome X or dyslipidemia in a subject comprising administering to a subject a derivative according to any one of aspects 1a-52a or a pharmaceutical composition according to aspects 53a or 54a.
67a. A process for preparing a pharmaceutical composition according to aspect 53a or 54a comprising mixing a derivative according to any one of aspects 1a-52a with pharmaceutically acceptable substances and/or excipients.

In one aspect a derivative of human amylin or an analogue thereof is provided which shows high potency. In a further aspect a derivative of human amylin or an analogue thereof is provided which shows improved potency relative to human amylin. In a still further aspect a derivative of human amylin or an analogue thereof is provided which shows potency comparable to pramlintide. In a still further aspect a derivative of human amylin or an analogue thereof is provided which shows improved potency relative to pramlintide.

The term “potency” (or activity) is used to indicate the affinity to an amylin receptor of a derivative according to the invention and may be measured in e.g. a Luciferase assay as described in the assay section.

In one aspect a derivative of human amylin or an analogue thereof is provided which is physical stable. In a further aspect a derivative of human amylin or an analogue thereof is provided which has increased physical stability relative to human amylin.

The term “physical stability” of a derivative according to the invention refers as used herein to the tendency of the protein not to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations may be evaluated by means of visual inspection, ThT fibrillation assay and/or turbidity measurements as described elsewhere herein.

The term “human amylin” as used herein refers to the peptide human amylin having the sequence as depicted in SEQ ID No 17. The term includes, but is not limited to, a human peptide hormone of 37 amino acids referred to as amylin, which is co-secreted with insulin from β-cells of the pancreas. Human amylin has the following amino acid sequence: LysCys-Asn-Thr-Ala-Thr-Cys-Ala-Thr-Gln-Arg-Leu-Ala-Asn-Phe-Leu-Val-His-Ser-Ser-Asn-AsnPhe-Gly-Ala-Ile-Leu-Ser-Ser-Thr-Asn-Val-Gly-Ser-Asn-Thr-Tyr (SEQ ID NO:17). Thus, the structural formula is Lys-Cys-Asn-Thr-Ala-Thr-Cys-Ala-Thr-Gln-Arg-Leu-Ala-Asn-Phe-LeuVal-His-Ser-Ser-Asn-Asn-Phe-Gly-Ala-Ile-Leu-Ser-Ser-Thr-Asn-Val-Gly-Ser-Asn-Thr-Tyr-NH₂ (SEQ ID NO: 17) as shown below with a disulfide bridge between the two Cys residues and a C-terminal amide group.

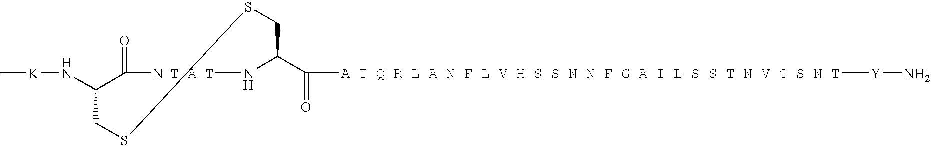

In the present text, the term “analogue of human amylin” is used to designate a peptide wherein one or more amino acid residues of human amylin of SEQ ID NO:17 independently have been substituted by other amino acid residues and/or wherein one or more amino acid residues of human amylin have been deleted and/or wherein one or more amino acid residues have been added to human amylin. In one aspect a substitution or addition is with any natural amino acid. Amylin analogs also include amylin having insertions, deletions, and/or substitutions in at least one or more amino acid positions of SEQ ID NO: 17. The number of amino acid insertions, deletions, or substitutions may be at least 1, 2, 3, 4, 5, 6, 7, 8, 9, 10 11 or 12. Insertions or substitutions may be with other natural or unnatural amino acids, synthetic amino acids, peptidomimetics, or other chemical compounds.

When used herein the term “natural amino acid” is an amino acid (with the usual three letter codes & one letter codes in parenthesis) selected from the group consisting of: Glycine (Gly & G), proline (Pro & P), alanine (Ala & A), valine (Val & V), leucine (Leu & L), isoleucine (Ile & I), methionine (Met & M), cysteine (Cys & C), phenylalanine (Phe & F), tyrosine (Tyr & Y), tryptophan (Trp & W), histidine (H is & H), lysine (Lys & K), arginine (Arg & R), glutamine (Gln & Q), asparagine (Asn & N), glutamic acid (Glu & E), aspartic acid (Asp & D), serine (Ser & S) and threonine (Thr & T). If, due to typing errors, there are deviations from the commonly used codes, the commonly used codes apply. The amino acids present in the insulins of this invention are, preferably, amino acids which can be coded for by a nucleic acid.

In one aspect of the invention, addition or deletion of amino acid residues can take place at the N-terminal of the peptide and/or at the C-terminal of the peptide.

In one aspect substitution or addition of amino acid residues comprises substitution and/or addition of glutamic acid residue(s), lysine residue(s), arginine residue(s), histidine residue(s) and/or aspartic acid residue(s) to obtain an amylin analogue having 0-8 additional charges compared to the parent amylin. In a further aspect substitution or addition of amino acid residues comprises substitution and/or addition of glutamic acid residue(s), arginine residue(s), and/or aspartic acid residue(s) to obtain an amylin analogue having 0-8 additional charges compared to the parent amylin. In a yet further aspect substitution or addition of amino acid residues comprises substitution and/or addition of glutamic acid residue(s) and/or arginine residue(s) to obtain an amylin analogue having 0-8 additional charges compared to the parent amylin. In a still further aspect substitution or addition of amino acid residues comprises substitution and/or addition of glutamic acid residue(s) to obtain an amylin analogue having 0-8 additional charges compared to the parent amylin.

An analogue of human amylin has in one aspect preferably from 30 to 45 naturally occurring or non-naturally occurring amino acids, preferably from 35-40 naturally occurring or non-naturally occurring amino acids.

The term “derivative” is used in the present text to designate a peptide in which one or more of the amino acid residues of the peptide have been modified, e.g. by alkylation, acylation, ester formation, amide formation or by maleimide coupling.

The term “a derivative of human amylin or an analogue thereof” or “an amylin derivative” is used in the present text to designate a derivative of human amylin or a derivative of an analogue of human amylin.

In a further aspect of the invention, an analogue of human amylin has sufficient homology with human amylin such that it exhibits at least 70% in vivo activity compared to human amylin.

The derivatives of the present invention may be capable of binding to or otherwise directly or indirectly interacting with an amylin receptor, or other receptor or receptors with which amylin itself may interact to elicit a biological response, e.g., reducing food intake. Derivatives of the invention may bind an amylin receptor with an affinity better than 20 nM, 10 nM, or 5 nM, and more preferably with an affinity better than 2 nM, 1 nM, 0.5 nM, 0.2 nM or 0.10 nM.

In one aspect according to the invention, the derivatives of the present invention may retain at least about 25%, preferably about 30%, 40%, 50%, 60%, 70%, 80%, 90%, 95%, 98%, or 99% percent of the biological activity of amylin. In another aspect, derivatives of the invention exhibit improved biological activity. In a further aspect, derivatives of the present invention exhibit at least about 110%, 125%, 130%, 140%, 150%, 200%, or more of the biological activity of amylin.

The term “activity” refers in one aspect to the ability to reduce appetite and/or increase satiety. In one aspect of the invention, activity is measured by the ability to reduce appetite as e.g. described in Pharmacological assays I and II under the heading “ASSAYS”.

In one aspect according to the invention, the derivatives of the present invention may demonstrate an ability to reduce cumulative food intake more than 5% over administration of the vehicle, preferably more than 15%, more preferably more than 25%, even more preferably more than 35% or 40% most preferably more than 50% over the vehicle.

The term “time of action” refers in the present context to the time span where a pharmacological effect such as reduced food intake is measurable.

In one aspect according to the invention, the amylin derivative is less likely to fibrillate in vivo and/or ex-vivo compared to human amylin. The tendency of fibrillation may e.g. be estimated in a Thioflavin T test as described below under the definition of the term “physical stability”.

In one aspect an amylin derivative according to the invention has an increased tendency to form an alfa helical containing conformation compared to pramlintide under similar conditions. The content of alfa helix is estimated by the magnitude of the negative molar circular dichroism signal at 220 nm, and further assigned by the classical CD spectral signatures for an alfa helix: A positive peak around 190 nm and a negative peak around 208 nm.

In one aspect according to the invention, the amylin derivative has a protracted pharmaco-kinetic profile compared to pramlintide as measured by standard procedures such as ELISA known to people skilled in the art. In another aspect of the invention, the amylin derivative has a plasma T½ of at least 1 hour. In another aspect, of the invention the plasma T½ is at least 1.5 hour. In another aspect of the invention, the amylin derivative has a plasma T½ of at least 2 hours. In another aspect of the invention, the amylin derivative has a plasma T½ of at least 4 hours. In another aspect of the invention, the amylin derivative has a plasma T½ of at least 6 hours. In another aspect of the invention, the amylin derivative has a plasma T½ of at least 8 hours. In another aspect of the invention, the amylin derivative has a plasma T½ of at least 12 hours. In another aspect of the invention, the amylin derivative has a plasma T½ of at least 16 hours. In another aspect of the invention, the amylin derivative has a plasma T½ of at least 24 hours. In one aspect of the invention, the amylin derivative has a plasma T½ of at least 48 hours. In one aspect of the invention, the amylin derivative has a plasma T½ of at least 96 hours.

In another further aspect of the invention, an analogue of human amylin has the amino acid sequence of human amylin modified so that from one, two, three, four, five, six, seven, eight, nine, ten, eleven, twelve, thirteen, fourteen, fifteen, sixteen, seventeen, eighteen, nineteen, twenty, twenty-one or twenty-two amino acids differ from the amino acid in the corresponding position of human amylin.

In another further aspect of the invention, an analogue comprises 22 or less than 22 modifications (substitutions, deletions, additions) relative to human amylin. In another further aspect of the invention, an analogue comprises less than 21 modifications (substitutions, deletions, additions) relative to human amylin. In another further aspect of the invention, an analogue comprises less than 20, less than 19, less than 18, less than 17, less than 16, less than 15, less than 15, less than 13, less than 12, less than 11, less than 10 modifications (substitutions, deletions, additions) relative to human amylin. In another further aspect of the invention, an analogue comprises less than 9 modifications (substitutions, deletions, additions) relative to human amylin. In another further aspect of the invention, an analogue comprises less than 8 modifications (substitutions, deletions, additions) relative to human amylin. In another further aspect of the invention, an analogue comprises less than 7 modifications (substitutions, deletions, additions) relative to human amylin. In another further aspect of the invention, an analogue comprises less than 6 modifications (substitutions, deletions, additions) relative to human amylin. In another further aspect of the invention, an analogue comprises less than 5 modifications (substitutions, deletions, additions) relative to human amylin. In another further aspect of the invention, an analogue comprises less than 4 modifications (substitutions, deletions, additions) relative to human amylin. In another further aspect of the invention, an analogue comprises less than 3 modifications (substitutions, deletions, additions) relative to human amylin. In another further aspect of the invention, an analogue comprises less than 2 modifications (substitutions, deletions, additions) relative to human amylin. In another further aspect of the invention, an analogue comprises only a single modification (substitutions, deletions, additions) relative to human amylin.

In one aspect of the invention the an analogue of human amylin has the amino acid sequence of human amylin modified so that 12 amino acid residues have been substituted or deleted.

In one aspect of the invention the an analogue of human amylin has the amino acid sequence of human amylin modified so that 12 amino acid residues have been substituted or deleted and the N-terminal has been extended with from 1-10 amino acid residues.

In one aspect of the invention the an analogue of human amylin has the amino acid sequence of human amylin modified so that 11, 10, 9, 8, 7, 6, 5, 4, 3, 2 or 1 amino acid residues have been substituted or deleted.

In one aspect of the invention the an analogue of human amylin has the amino acid sequence of human amylin modified so that 11, 10, 9, 8, 7, 6, 5, 4, 3, 2 or 1 amino acid residues have been substituted or deleted and the N-terminal has been extended with from 1, 2, 3, 4, 5, 6, 7, 8, 9 or 10 amino acid residues.

In one aspect of the invention, the modification(s) are substitution(s). In one aspect of the invention, the modification(s) are deletion(s). In one aspect of the invention, the modification(s) are addition(s).

In one aspect a derivative of human amylin or an analogue thereof is extended in the N-terminal. In a further aspect a derivative of human amylin or an analogue thereof is extended in the N-terminal, where the N-terminal extension consists of 1-10 amino acids, 1-8 amino acids or 1-6 amino acids. In a further aspect a derivative of human amylin or an analogue thereof is extended in the N-terminal, where the N-terminal extension consists of 5 amino acids, 4 amino acids, 3 amino acids, 2 amino acids or 1 amino acid. In a further aspect the N-terminal extension is further linked to an albumin binding residue, optionally via a linker.

In one aspect the invention relates to a derivative of human amylin with SEQ ID No. 17.

In another aspect the invention relates to a derivative of a human amylin analogue.

In another aspect of the invention, the derivative has from 1-12 amino acid substitutions compared to human amylin.

In another aspect of the invention, the derivative has from 1-6 amino acid substitutions compared to human amylin.

In another aspect of the invention, the derivative has from 1-5 amino acid substitutions compared to human amylin.

In another aspect of the invention, the derivative has from 1-4 amino acid substitutions compared to human amylin.

In another aspect of the invention, the derivative has from 1-3 amino acid substitutions compared to human amylin.

In another aspect of the invention, the derivative has from 1-2 amino acid substitutions compared to human amylin.

In another aspect the invention relates to a derivative of a human amylin analogue which is further modified according to the invention, wherein the human amylin analogue is 25Pro 28Pro 29Pro human amylin (SEQ ID NO: 1):

(SEQ ID NO: 1)

Lys-Cys-Asn-Thr-Ala-Thr-Cys-Ala-Thr-Gln-Arg-Leu-
Ala-Asn-Phe-Leu-Val-His-Ser-Ser-Asn-Asn-Phe-Gly-
Pro-Ile-Leu-Pro-Pro-Thr-Asn-Val-Gly-Ser-Asn-Thr-
Tyr.

The invention relates in another aspect to a derivative, wherein the albumin binding residue, optionally via a linker is linked via the ε-amino group of the lysine residue.

The invention relates in another aspect to a derivative, wherein the albumin binding residue or the polyethylene glycol polymer, optionally via a linker is linked via the alpha-amino group of the lysine residue.

The invention relates in another aspect to a derivative, wherein the lysine residue is linked to an albumin binding residue via a linker.

The term “linked to” as used herein means chemically connected via a covalent bond. For example a lysine residue is linked to an albumin binding residue via a chemical bond. Such a chemical bond can as an example be obtained by derivatisation of an epsilon amino group of lysine by acylation with an active ester of an albumin binding residue such as a long fatty acid.

Other examples of connecting two chemical moieties as used in the present invention includes but is not limited to alkylation, ester formation, amide formation or maleimide coupling.

The term “albumin binding residue” as used herein means in one aspect of the invention a residue which binds non-covalently to human serum albumin. The albumin binding residue connected to the therapeutic polypeptide typically has an affinity (binding constant) below 10 μM to human serum albumin and preferably below 1 μM. A range of albumin binding residues are known among linear and branched lipohophillic moieties containing 4-40 carbon atoms, compounds with a cyclopentanophenanthrene skeleton, a tetrazol, peptides having 10-30 amino acid residues etc.

Albumin binding affinity may be determined by several methods known within the art.

In one method the derivative to be measured is radiolabeled with e.g. ¹²⁵I or ³H and incubated with immobilized albumin (Kurtzhals et. al., Biochem. J., 312, 725-731 (1995)). The binding of the derivative relative to a standard is calculated. In another method a related compound is radiolabeled and its binding to albumin immobilized on e.g. SPA beads is competed by a dilution series of the derivative to be measured. The EC50 value for the competition is a measure of the affinity of the derivative. In a third method, the receptor affinity or potency of a derivative is measured at different concentrations of albumin, and the shift in relative affinity or potency of the derivative as a function of albumin concentration reflects its affinity for albumin.

In one aspect of the invention the albumin binding residue comprises a group which can be negatively charged.

The term “C₁₋₆-alkyl” as used herein means a saturated, branched, straight or cyclic hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl, cyclohexane and the like.

The term “linker” or “spacer” (the two terms spacer and linker is used interchangeably in the present specification) as used herein means a chemical moiety that separates a peptide and an albumin binding residue or a polyethylene glycol polymer.

The term “hydrophilic linker” or “hydrophilic spacer” as used herein means a linker/spacer that separates a peptide and an albumin binding residue with a chemical moiety which comprises at least 5 heavy atoms where 30-50% of these are either N or O.

In one aspect of the invention, the linker comprises 1 or more negatively charged entities to obtain an amylin derivative according to the invention wherein 0-8 additional charges have been added to the molecule compared to the parent amylin.

In one aspect of the invention, the linker comprises an unbranched alkane α,ω-dicarboxylic acid group having from 1 to 7 methylene groups, preferably two methylene groups which linker forms a bridge between an amino group of the peptide and an amino group of the albumin binding residue.

In another aspect of the invention, the linker comprises one or more alkylene glycol units, such as 1 to 5 alkylene glycol units. The alkylene glycol units are in a further aspect ethylene glycol, propylene glycol or butylene glycol but can also be higher alkylene glycols.

In another aspect of the invention, the linker is a hydrophilic linker selected from

—(CH₂)ₗD[(CH₂)ₙE]ₘ(CH₂)ₚ-Q_{q}-, wherein

l, m and n independently are 1-20 and p is 0-10,

Q is —Z—(CH₂)ₗD[(CH₂)ₙG]ₘ(CH₂)ₚ—,

q is an integer in the range from 0 to 5,

each D, E, and G are independently selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,

Z is selected from —C(O)NH—, —C(O)NHCH₂—, —OC(O)NH —C(O)NHCH₂CH₂—, —C(O)CH₂—, —C(O)CH═CH—, —(CH₂)ₛ—, —C(O)—, —C(O)O— or —NHC(O)—, wherein s is 0 or 1.

In another aspect of the invention, the linker is a hydrophilic linker as defined above wherein l is 1 or 2, n and m are independently 1-10 and p is 0-10.

In another aspect of the invention, the linker is a hydrophilic linker as defined above wherein D is —O—.

In another aspect of the invention, the linker is a hydrophilic linker as defined above wherein E is —O—.

In yet another aspect of the invention, the hydrophilic linker is

—CH₂O[(CH₂)₂O]ₘ(CH₂)ₚQ_{q}-, wherein m is 1-10, p is 1-3, and Q is —Z—CH₂O[(CH₂)₂O]ₘ(CH₂)ₚ wherein Z is as defined above.

In another aspect of the invention, the linker is a hydrophilic linker as defined above wherein q is 1.

In another aspect of the invention, the linker is a hydrophilic linker as defined above wherein G is —O—.

In another aspect of the invention, the linker is a hydrophilic linker as defined above wherein Z is selected from the group consisting of —C(O)NH—, —C(O)NHCH₂—, and —OC(O)NH—.

In another aspect of the invention, the linker is a hydrophilic linker as defined above wherein q is 0.

In another aspect of the invention, the linker is a hydrophilic linker as defined above wherein l is 2.

In another aspect of the invention, the linker is a hydrophilic linker as defined above wherein n is 2.

In one aspect of this invention a “hydrophilic linker” B is used that separates a peptide and an albumin binding residue with a chemical moiety.

In one aspect of this invention, the hydrophilic linker B is

—C(O)—(CH₂)ₗ—O—[(CH₂CH₂—O]ₘ—(CH₂)ₚ—[NHC(O)—(CH₂)ₗ—O—[(CH₂)ₙ—O]ₘ—(CH₂)ₚ]_{q}—NH—,

wherein l, m, n, and p independently are 1-5, and q is 0-5.

In yet another aspect of this invention, the hydrophilic linker B is

—C(O)—CH₂—O—CH₂CH₂—O—CH₂CH₂ [NHC(O)—CH₂—O—CH₂CH₂O—CH₂CH₂]_{q}—NH—,

wherein q is 0-5.

In yet another aspect of this invention, the hydrophilic linker B is

—C(O)—CH₂—O—CH₂CH₂—O—CH₂CH₂—NHC(O)—CH₂—O—CH₂CH₂O—CH₂CH₂—NH—.

In yet another aspect of the invention, the hydrophilic linker B is —[CH₂CH₂O]ₘ₊₁(CH₂)ₚQ_{q}-

wherein m and p independently is 0-10, q is 0-5 and

Q is —Z—(CH₂)ₗD[(CH₂)ₙG]ₘ(CH₂)ₚ— as defined above.

In yet another aspect of the invention, the hydrophilic linker B is

—(CH₂)ₗ—O—[(CH₂)ₙ—O]ₘ—(CH₂)ₚ-[C(O)NH—(CH₂)ₗ—O—[(CH₂)ₙ—O]ₘ—(CH₂)ₚ]_{q}—,

wherein l, m, n, and p independently are 1-5, and q is 0-5.

In a further aspect of the invention, the linker comprises an amino acid residue except Cys, or a dipeptide such as Gly-Lys, Glu-Glu or Glu-Arg, or a tripeptide such as Gly-Gly-Lys, Glu-Glu-Glu or Glu-Glu-Arg.

Suitable PEG polymers are typically commercially available or may be made by techniques well-known to those skilled in the art.

In one aspect of the invention, the PEG polymer has a molecular weight of greater than 700 D, in a further aspect a molecular weight greater than 5 kD, in yet a further aspect greater than 10 kD, and in a even further aspect greater that 20 kD. The PEG polymer may be linear or branched. In cases where the PEG polymer is greater than 20 KDa, the PEG polymer is preferable having a branched structure, such as for example, a 43 kD branched PEG-peptide (Shearwater 2001 catalog #2D3XOT01, mPEG2-MAL).

The attachment of a PEG on an intact peptide can be accomplished by attaching the PEG on the opposite side of the peptide surface that interacts with the receptor.

In one aspect of the invention, the attachment of PEG will occur on the amino acid residue in position 2, 3, 4, 5, or 6 of human amylin or an analog thereof which is substituted with a lysine residue or a cysteine, optionally via a linker. In a further aspect of the invention, the attachment of PEG will occur on the amino acid residue in position 23, 24, 25, 26, 27 or 28 of human amylin or an analog thereof which is substituted with a lysine residue or a cysteine, optionally via a linker. In yet a further aspect of the invention, the attachment of PEG will occur on the amino acid residue in position 32, 33, 34, 35, 36, or 37 of human amylin or an analog thereof which is substituted with a lysine residue or a cysteine, optionally via a linker.

There are several strategies for coupling PEG to peptides (see, e.g. Veronese, Biomaterials 22:405-417, 2001), all of which are incorporated herein by reference in their entirety. Those skilled in the art, will therefore be able to utilize well-known techniques for linking the PEG polymer to human amylin or the amylin analogs described herein.

Briefly, cysteine PEGylation is one method for site-specific PEGylation, and can be accomplished by introducing a unique cysteine mutation at one of the specific positions on human amylin or the amylin analog and then reacting the resulting peptide with a cysteine-specific PEGylation reagent, such as PEG-maleimide. It may be necessary to mutate the peptide in order to allow for site-specific PEGylation. For example, if the peptide contains cysteine residues, these will need to be substituted with conservative amino acids in order to ensure site-specific PEGylation. In addition, rigid linkers, including but not limited to “GGS”, “GGSGGS”, and “PPPS” may be added to the C-terminus, but before the site of PEG attachment (i.e. a unique cysteine residue).

In one aspect of the invention, the albumin binding residue is a lipophilic residue. In a further aspect, the lipophilic residue is connected to a lysine residue optionally via a linker by conjugation chemistry such as by alkylation, acylation, ester formation, or amide formation or to a cysteine residue by maleimide coupling.

In a further aspect of the invention, the albumin binding residue is negatively charged at physiological pH. In another aspect of the invention, the albumin binding residue comprises a group which can be negatively charged. One preferred group which can be negatively charged is a carboxylic acid group.

In a further aspect of the invention, the albumin binding residue binds non-covalently to albumin. In another aspect of the invention, the albumin binding residue has a binding affinity towards human serum albumin better than about 10 μM or better than about 1 μM.

In yet another aspect of the invention, the albumin binding residue is selected from the group consisting of a straight chain alkyl group, a branched alkyl group, a group which has an ω-carboxylic acid group, and a partially or completely hydrogenated cyclopentanophenanthrene skeleton.

In one aspect of the invention the albumin binding residue comprises tetrazole. In one aspect the albumin binding residue is (CH₂)₁₋₂₀-tetrazoyl. In one aspect the albumin binding residue is (CH₂)₁₅-tetrazoyl.

In a further aspect of the invention, the albumin binding residue is a cibacronyl residue.

In a further aspect of the invention, the albumin binding residue has from 6 to 40 carbon atoms, from 8 to 26 carbon atoms or from 8 to 20 carbon atoms.

In a further aspect of the invention, the albumin binding residue is an acyl group selected from the group comprising CH₃(CH₂)ᵣCO—, wherein r is an integer from 4 to 38, preferably an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In another aspect of the invention, the albumin binding residue is an acyl group of a straight-chain or branched alkane α,ω-dicarboxylic acid.

In another aspect of the invention, the albumin binding residue is an acyl group selected from the group comprising HOOC(CH₂)ₛCO—, wherein s is an integer from 4 to 38, preferably an integer from 4 to 24, more preferred selected from the group comprising HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

In another aspect of the invention, the albumin binding residue is a group of the formula CH₃(CH₂)ᵥCO—NHCH(COOH)(CH₂)₂CO—, wherein v is an integer from 10 to 24.

In another aspect of the invention, the albumin binding residue is a group of the formula CH₃(CH₂)_{w}CO—NHCH((CH₂)₂COOH)CO—, wherein w is an integer from 8 to 24.

In another aspect of the invention, the albumin binding residue is a group of the formula COOH(CH₂)ₓCO— wherein x is an integer from 8 to 24.

In another aspect of the invention, the albumin binding residue is a group of the formula —NHCH(COOH)(CH₂)₄NH—CO(CH₂)_{y}CH₃, wherein y is an integer from 8 to 18.

In one aspect of this invention, the combined albumin binding residue and linker is

—C(O)—(CH₂)ₗ—O—[(CH₂CH₂—O]ₘ—(CH₂)ₚ—[NHC(O)—(CH₂)ₗ—O—[(CH₂)ₙ—O]ₘ—(CH₂)ₚ]_{q}—NH—W,

wherein l, m, n, and p independently are 1-5, and q is 0-5

and W is selected from the group consisting of

CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO—, CH₃(CH₂)₂₂CO—HOOC(CH₂)₁₄CO—, HOOC(CH₂)₁₆CO—, HOOC(CH₂)₁₀CO—, HOOC(CH₂)₂₀CO— and HOOC(CH₂)₂₂CO—.

In one aspect of this invention, the combined albumin binding residue and linker is

—C(O)—CH₂—O—CH₂CH₂—O—CH₂CH₂[NHC(O)—CH₂—O—CH₂CH₂—O—CH₂CH₂]_{q}—NH—Ac,

wherein q is 0-5 and wherein Ac is acetyl.

In another aspect of this invention, the combined albumin binding residue and linker is a group of the formula

—C(O)—CH₂—O—CH₂CH₂—O—CH₂CH₂—NHC(O)—CH₂—O—CH₂CH₂O —CH₂CH₂—NH—Ac,

wherein Ac is acetyl.

In another aspect of this invention, the combined albumin binding residue and linker is

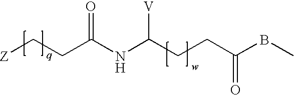

wherein B is defined as described above, and Z, V, q and w is defined as below.

In another aspect of this invention, the combined albumin binding residue and linker is

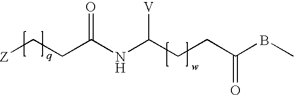

wherein Z is CH₃ or COOH, V is H or COOH, q is 7 to 21, w is 0 to 5, and k is 0 to 5 and B is —C(O)—(CH₂)ₗ—O—[(CH₂CH₂—O]ₘ—(CH₂)ₚ—[NHC(O)—(CH₂), —O—[(CH₂)ₙ—O]ₘ—(CH₂)ₚ]_{q}—NH—,

wherein l, m, n, and p independently are 1-5, and q is 0-5.

In another aspect of this invention, the combined albumin binding residue and linker is

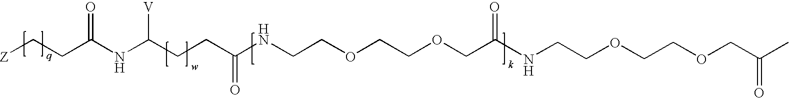

wherein Z is CH₃ or COOH, V is H or COOH, q is 7 to 21, w is 0 to 5, and k is 0 to 5. In another aspect of this invention, Z is CH₃ and V is COOH. In another aspect of this invention V is H and Z is COOH. In another aspect of this invention, V is H and Z is CH₃. In another aspect of this invention, V and Z are both COOH. In a preferred aspect of this invention, q is 13 to 19, and more preferable q is 13 to 15. In a preferred aspect of this invention, k is 1 to 4, and more preferable 1 to 2. In a preferred aspect of this invention, w is 1 to 4, and more preferable 1 to 2.

In another aspect of this invention, the combined albumin binding residue and linker is

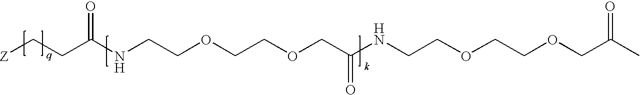

wherein Z is CH₃ or COOH, q is 7 to 21, and k is 0 to 5. In another aspect of this invention, Z is CH₃. In another aspect of this invention, Z is COOH. In a preferred aspect of this invention, q is 13 to 19, and more preferable q is 13, 14 or 15. In a preferred aspect of this invention, k is 1 to 4, and more preferable 1 or 2.

In another aspect of the invention, the albumin binding residue is a peptide, such as a peptide comprising less than 40 amino acid residues. A number of small peptides which are albumin binding residues as well as a method for their identification is found in J. Biol. Chem. 277, 38 (2002) 35035-35043.

In another aspect of the invention, the albumin binding residue optionally via a linker is connected via the ε-amino group of a lysine residue.

In a further aspect, the present invention relates to a amylin derivative wherein a lipophilic residue is connected to the parent peptide by means of a linker which is an amino acid residue except Cys, or is a dipeptide such as Gly-Lys, Glu-Arg or Glu-Glu, or is a tripeptide such as Gly-Gly-Lys, Glu-Glu-Arg or Glu-Glu-Glu or is a tetrapeptide such as Glu-Glu-Glu-Glu, Glu-Arg-Glu-Glu or is a multipeptide consisting of 5-10 amino acids, and wherein a carboxyl group of the parent peptide forms an amide bond with an amino group of a Lys residue or a dipeptide, tripeptide, tetrapeptide or multipeptide containing a Lys residue, and the other amino group of the Lys residue or a dipeptide, tripeptide or multipeptide containing a Lys residue forms an amide bond with a carboxyl group of the lipophilic residue.

In a further aspect, the present invention relates to a amylin derivative wherein a lipophilic residue is connected to the parent peptide by means of a linker which is an amino acid residue except Cys, or is a dipeptide such as Gly-Lys, Glu-Arg or Glu-Glu, or is a tripeptide such as Gly-Gly-Lys, Glu-Glu-Arg or Glu-Glu-Glu, or is a tetrapeptide such as Glu-Glu-Glu-Glu, Glu-Arg-Glu-Glu, or is a multipeptide consisting of 5-10 amino acids, and wherein an amino group of the parent peptide forms an amide bond with a carboxylic group of the amino acid residue or dipeptide, tripeptide, tetrapeptide or multipeptide linker, and an amino group of the amino acid residue or dipeptide, tripeptide or multipeptide linker forms an amide bond with a carboxyl group of the lipophilic residue.

In a further aspect, the present invention relates to a amylin derivative wherein a lipophilic residue is connected to the parent peptide by means of a linker which is an amino acid residue except Cys, or is a dipeptide such as Gly-Lys, Glu-Arg or Glu-Glu, or is a tripeptide such as Gly-Gly-Lys, Glu-Glu-Arg or Glu-Glu-Glu, or is a tetrapeptide such as Glu-Glu-Glu-Glu, Glu-Arg-Glu-Glu, or is a multipeptide consisting of 5-10 amino acids, and wherein a carboxyl group of the parent peptide forms an amide bond with an amino group of the amino acid residue linker or dipeptide, tripeptide, tetrapeptide or multipeptide linker, and the carboxyl group of the amino acid residue linker or dipeptide, tripeptide or multipeptide linker forms an amide bond with an amino group of the lipophilic residue.

In a further aspect, the present invention relates to a amylin derivative wherein a lipophilic residue is connected to the parent peptide by means of a linker which is an amino acid residue except Cys, or is a dipeptide such as Gly-Lys, Glu-Arg or Glu-Glu, or is a tripeptide such as Gly-Gly-Lys, Glu-Glu-Arg or Glu-Glu-Glu, or is a tetrapeptide such as Glu-Glu-Glu-Glu, Glu-Arg-Glu-Glu, or is a multipeptide consisting of 5-10 amino acids, and wherein a carboxyl group of the parent peptide forms an amide bond with an amino group of a linker which is Asp or Glu, or a dipeptide, tripeptide, tetrapeptide or multipeptide linker containing an Asp or Glu residue, and a carboxyl group of the linker forms an amide bond with an amino group of the lipophilic residue.

In one aspect of the invention, the C-terminal of the amylin derivative may be terminated as either an acid or amide. In a preferred aspect, the C-terminal of the amylin derivative is an amide.

In a further aspect, the present invention relates to an amylin derivative wherein a lipophilic residue is connected to the parent peptide by means of a N-terminal extension containing a lysine residue which is Lys, or is a dipeptide such as Gly-Lys, or is a tripeptide such as Gly-Gly-Lys, or is a tetrapeptide such as Glu-Glu-Glu-Glu, Glu-Arg-Glu-Glu, or is a multipeptide consisting of 5-10 amino acids having a Lys in the N-terminal position, and wherein an amino group of the Lys residue or the N-terminal Lys residue of the dipeptide, tripeptide, tetrapeptide or multipeptide containing a Lys residue forms an amide bond with a carboxyl group of the lipophilic residue. In one aspect the amide bond is between the ε-amino group of the lysine residue and the lipophilic residue. In one aspect the amide bond is between the α-amino group of the lysine residue and the lipophilic residue.

In a further aspect, the present invention relates to a amylin derivative wherein a lipophilic residue is connected to the parent peptide by means of a N-terminal extension which is an amino acid residue, or is a dipeptide such as Gly-Lys, Glu-Arg or Glu-Glu, or is a tripeptide such as Gly-Gly-Lys, Glu-Glu-Arg or is a tetrapeptide such as Glu-Glu-Glu, Glu-Glu-Glu-Glu, Glu-Arg-Glu-Glu, or is a multipeptide consisting of 5-10 amino acids, and wherein an amino group of the N-terminal amino acid of said N-terminal extension forms an amide bond with a carboxyl group of the lipophilic residue.

In a further aspect, the present invention relates to a amylin derivative wherein a lipophilic residue is connected to the parent peptide by means of a N-terminal extension which is an amino acid residue, or is a dipeptide such as Gly-Lys, Glu-Arg or Glu-Glu, or is a tripeptide such as Gly-Gly-Lys, Glu-Glu-Arg or is a tetrapeptide such as Glu-Glu-Glu, Glu-Glu-Glu-Glu, Glu-Arg-Glu-Glu, or is a multipeptide consisting of 5-10 amino acids, and wherein a carboxyl group of the N-terminal amino acid of said N-terminal extension forms an amide bond with an amino group of the lipophilic residue.

In one aspect of the invention, the amino acid sequence of the amylin derivative is selected from the group consisting of:

(SEQ ID No. 1)

KCNTATCATQRLANFLVHSSNNFGPILPPTNVGSNTY,

(SEQ ID No. 2)

KCKTATCATQRLANFLVHSSNNFGPILPPTNVGSNTY,

(SEQ ID No. 3)

KCNTATCATQRLANFLVHSSNNFGPILPKTNVGSNTY,

(SEQ ID No. 4)

KCNTATCATQRLANFLVHSSKNFGPILPPTNVGSNTY,

(SEQ ID No. 5)

KCNTATCATQRLANFLKHSSNNFGPILPPTNVGSNTY,

(SEQ ID No. 6)

KCNTATCATQRLANFLVHSSNNFGKILPPTNVGSNTY,

(SEQ ID No. 7)

KCNTATCATQRLANFLVRSSNNFGKILPPTNVGSNTY,

(SEQ ID No. 8)

CNTATCATQRLANFLVRSSNNLGPVLPPTNVGSNTY,

(SEQ ID No. 9)

KCKTATCATQRLANFLVPSSNNFGPILPPTNVGSNTY,

(SEQ ID No. 10)

KCNTATCATQRLANFLVRSSNNFGPILPPTNVGSNTY,

(SEQ ID No. 11)

KCKTATCATQRLANFLVRSSNNFGPILPPTNVGSNTY,

(SEQ ID No. 12)

KCGTATCATQRLANFLARSSNNFGPILSPTNVGSNTY,

(SEQ ID No. 13)

EERECNTATCATQRLANFLVHSSNNFGPILPPTNVGSNTY,

(SEQ ID No. 14)

KCKTATCATQRLANFLVRSSQNLGPVLPPTNVGSNTY,

(SEQ ID No. 15)

ECKTATCATQRLANFLVHSSNNFGPILPPTNVGSNTY,

(SEQ ID No. 16)

EECNTATCATQRLANFLVHSSNNFGPILPPTNVGSNTY.

(SEQ ID No. 17, human amylin)

KCNTATCATQRLANFLVHSSNNFGAILSSTNVGSNTY.

(SEQ ID No. 19)

EERECQTATCATQRLANFLVRSSQNLGPVLPPTNVGSNTY.

(SEQ ID No. 20)

EEEECQTATCATQRLANFLVRSSQNLGPVLPPTNVGSNTY.

(SEQ ID No. 21)

CKTATCATQRLANFLVHSSNNFGPILPPTNVGSNTY.

(SEQ ID No. 22)

CNTATCATQRLANFLVHSSNNFGPILPPTNVGSNTY.

(SEQ ID No. 23)

ECNTATCATQRLANFLVHSSNNFGPILPPTNVGSNTY.

(SEQ ID No. 24)

RCNTATCATQRLANFLVHSSNNFGPILPPTNVGSNTY.

(SEQ ID No. 25)

ERCNTATCATQRLANFLVHSSNNFGPILPPTNVGSNTY.

(SEQ ID No. 26)

RRCNTATCATQRLANFLVHSSNNFGPILPPTNVGSNTY.

(SEQ ID No. 27)

KKCNTATCATQRLANFLVHSSNNFGPILPPTNVGSNTY.

(SEQ ID No. 28)

RKCNTATCATQRLANFLVHSSNNFGPILPPTNVGSNTY.

(SEQ ID No. 29)

EKRKCNTATCATQRLANFLVHSSNNFGPILPPTNVGSNTY.

(SEQ ID No. 30)

EKRCNTATCATQRLANFLVHSSNNFGPILPPTNVGSNTY.

(SEQ ID No. 31)

EERECKTATCATQRLANFLVRSSQNLGPVLPPTNVGSNTY.

(SEQ ID No. 32)

ECQTATCATQRLANFLVRSSQNLGPVLPPTNVGSNTY

wherein at least one of the amino acid residue(s) or is linked to an albumin binding residue, optionally via a linker.

In a further aspect, only one lysine residue in an amylin derivative is linked to an albumin binding residue, optionally via a linker.

In a preferred aspect, the C-terminal of the amylin derivatives having any one of the amino acid sequences of SEQ ID No 1 to 16 is an amide.

In a further aspect of the invention, the albumin binding residue combined with a linker is selected from the group consisting of:

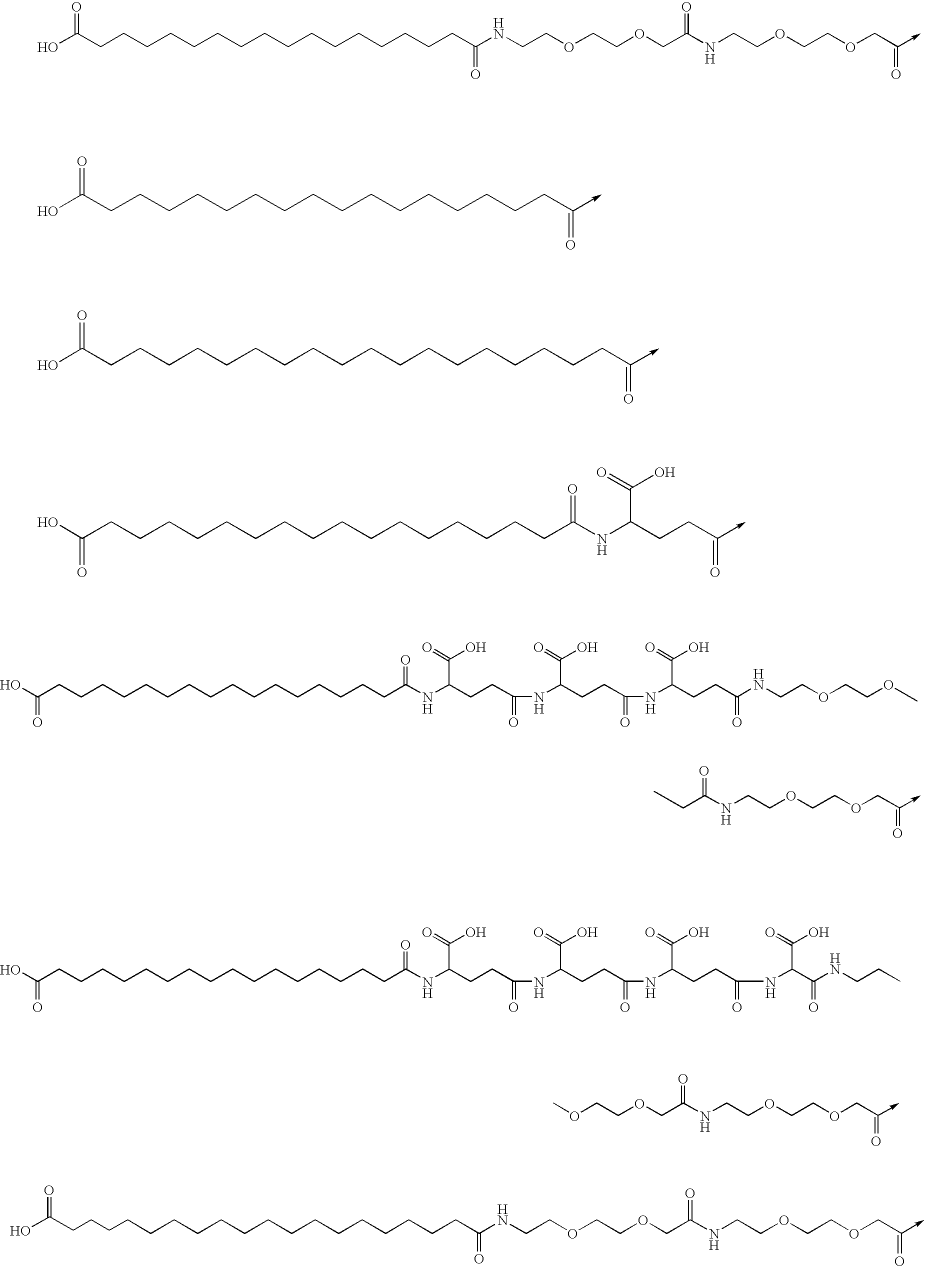

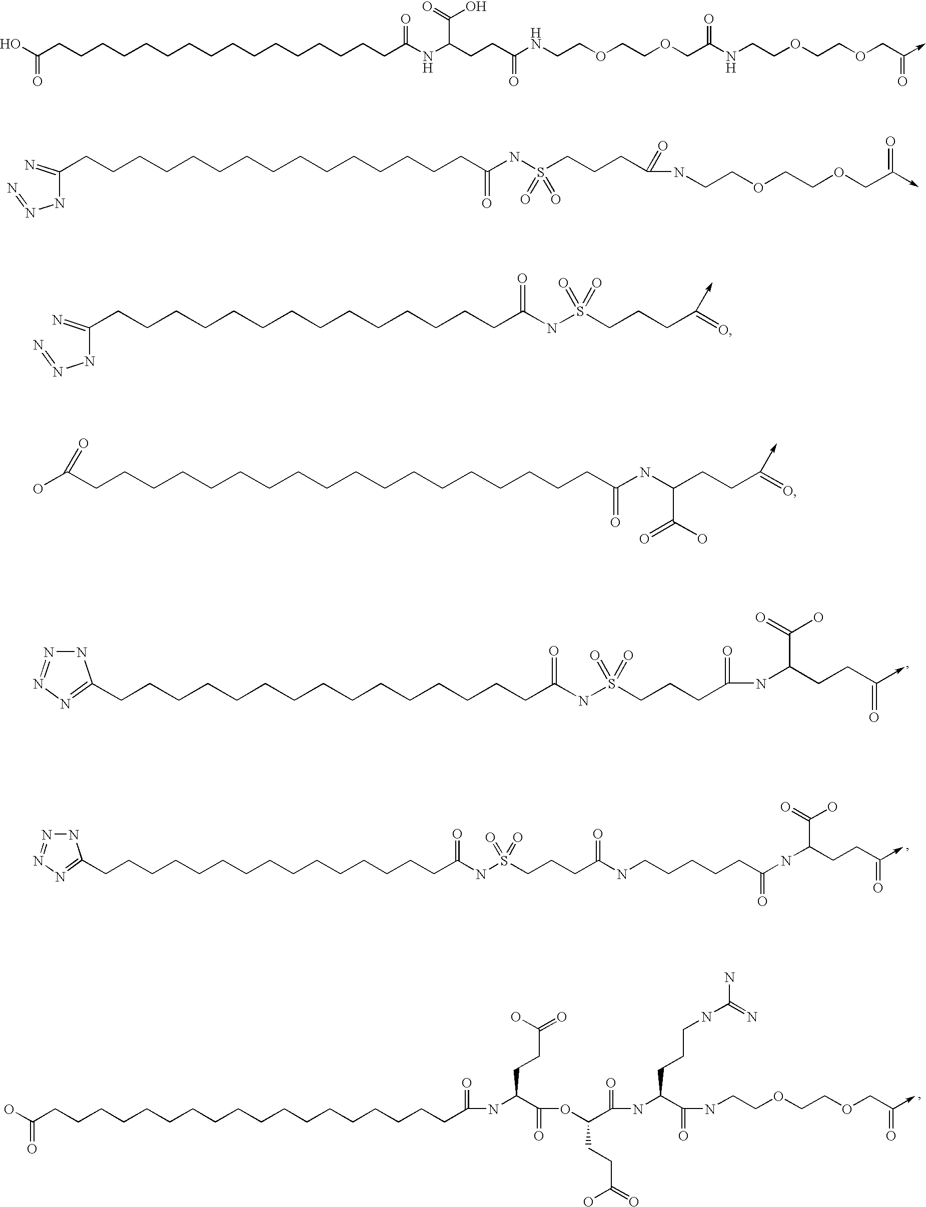

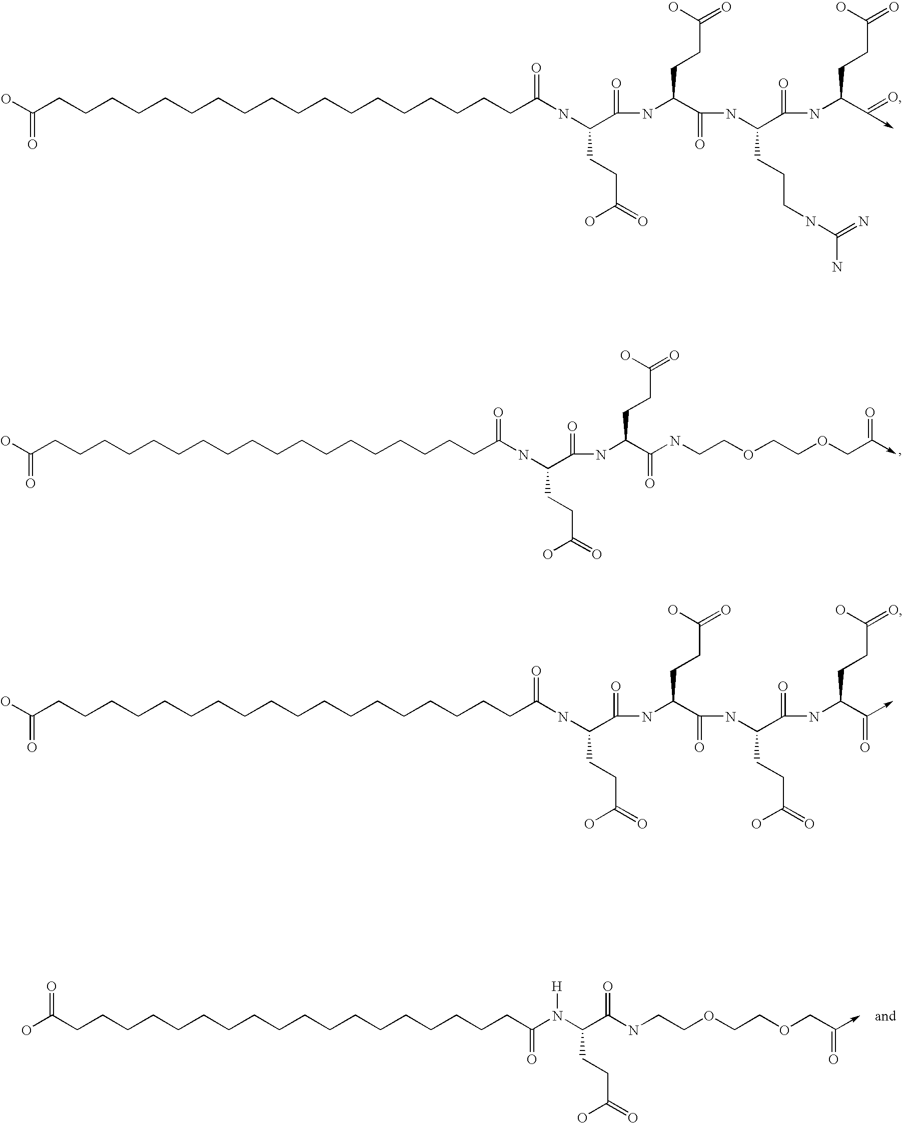

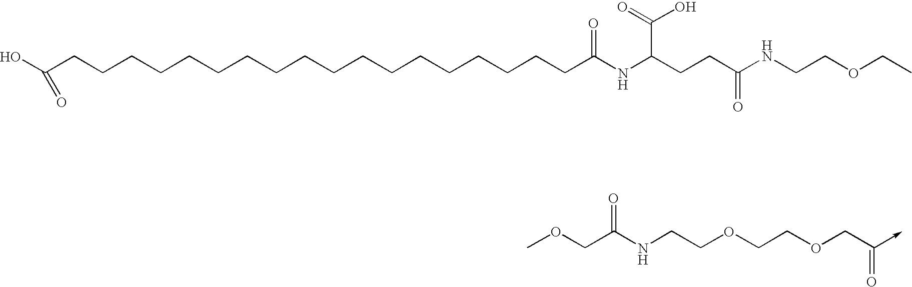

In one aspect of the invention, the amylin derivative is selected from the group consisting of:

KCKTATCATQRLANFLVHSSNNFGPILPPTNVGSNTY (SEQ ID No 2), wherein the lysine residue in position 3 is linked to an albumin binding residue or an albumin binding residue combined with a linker with a formula selected from the group consisting of:

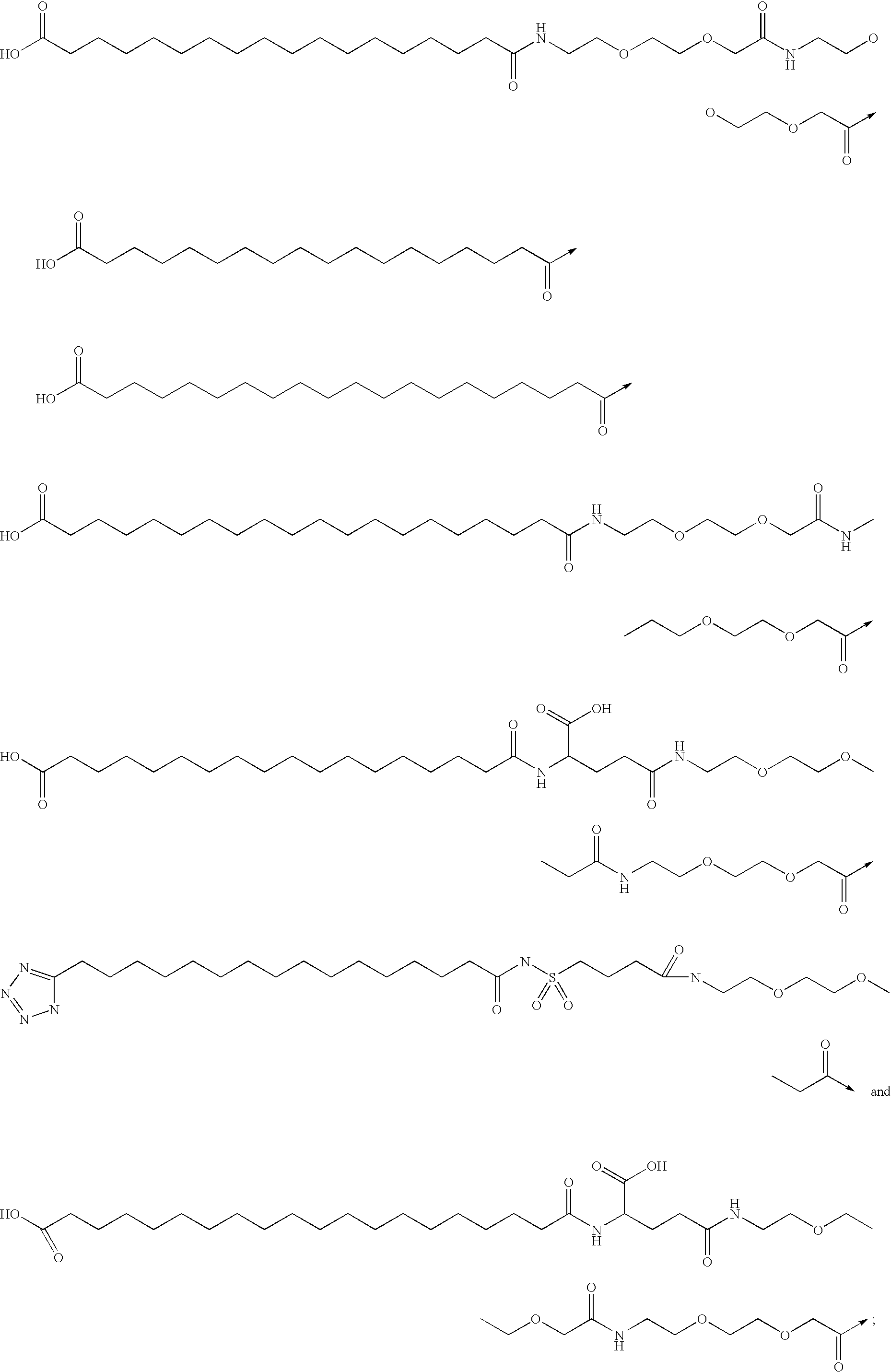

KCNTATCATQRLANFLVHSSNNFGPILPKTNVGSNTY (SEQ ID No 3), wherein the lysine residue in position 29 is linked to an albumin binding residue or an albumin binding residue combined with a linker with a formula selected from the group consisting of:

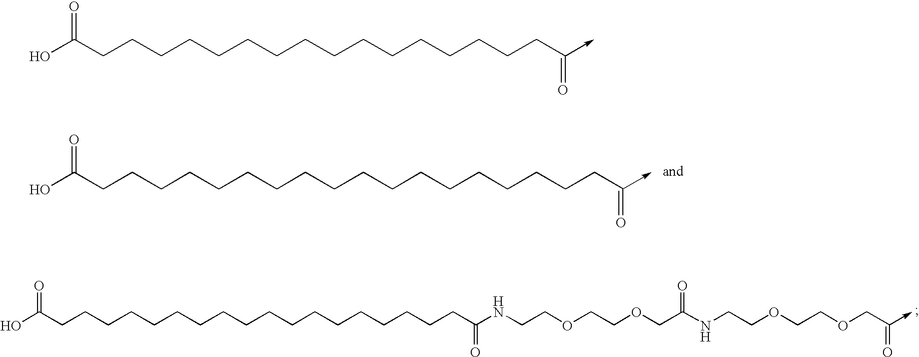

KCNTATCATQRLANFLVHSSKNFGPILPPTNVGSNTY (SEQ ID No 4), wherein the lysine residue in position 21 is linked to an albumin binding residue combined with a linker with the formula:

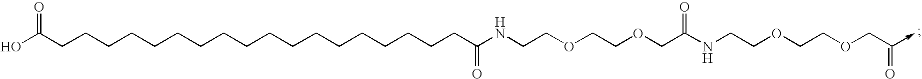

KCNTATCATQRLANFLKHSSNNFGPILPPTNVGSNTY (SEQ ID No 5), wherein the lysine residue in position 17 is linked to an albumin binding residue combined with a linker with the formula:

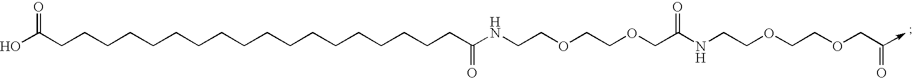

KCNTATCATQRLANFLVHSSNNFGKILPPTNVGSNTY (SEQ ID No 6), wherein the lysine residue in position 25 is linked to an albumin binding residue combined with a linker with a formula selected from the group consisting of:

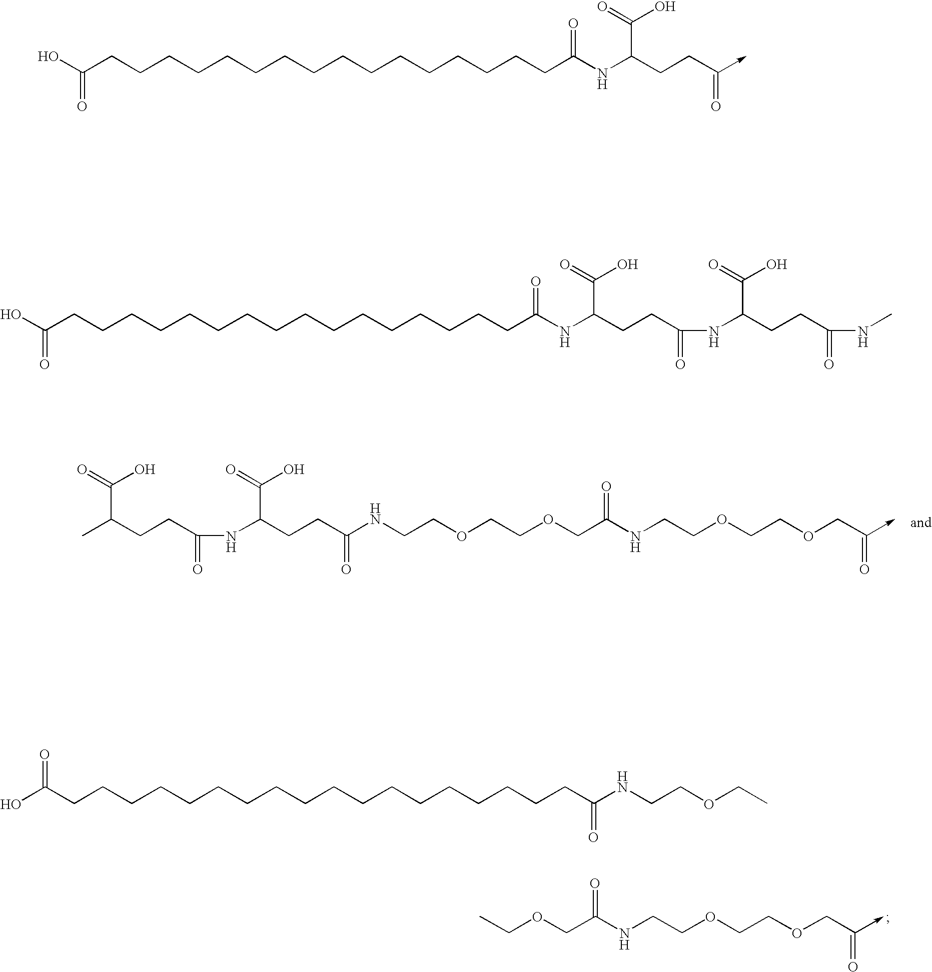

KCNTATCATQRLANFLVRSSNNFGKILPPTNVGSNTY (SEQ ID No 7), wherein the lysine residue in position 25 is linked to an albumin binding residue combined with a linker with the formula:

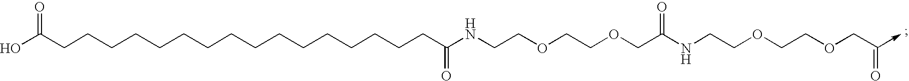

CNTATCATQRLANFLVRSSNNLGPVLPPTNVGSNTY (SEQ ID No 8), wherein the cysteine residue in position 1 is linked to an albumin binding residue or an albumin binding residue combined with a linker with a formula selected from the group consisting of:

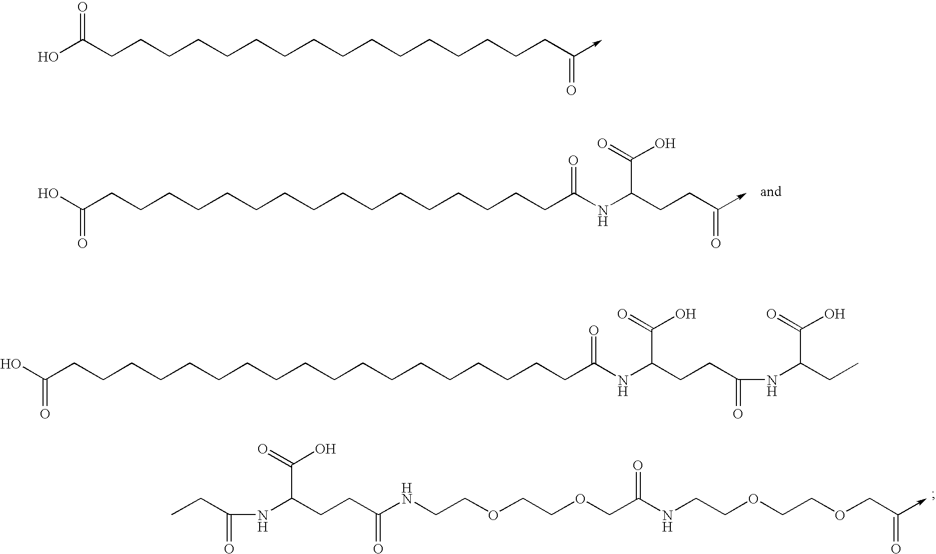

KCKTATCATQRLANFLVPSSNNFGPILPPTNVGSNTY (SEQ ID No 9), wherein the lysine residue in position 3 is linked to an albumin binding residue combined with a linker with a formula selected from the group consisting of:

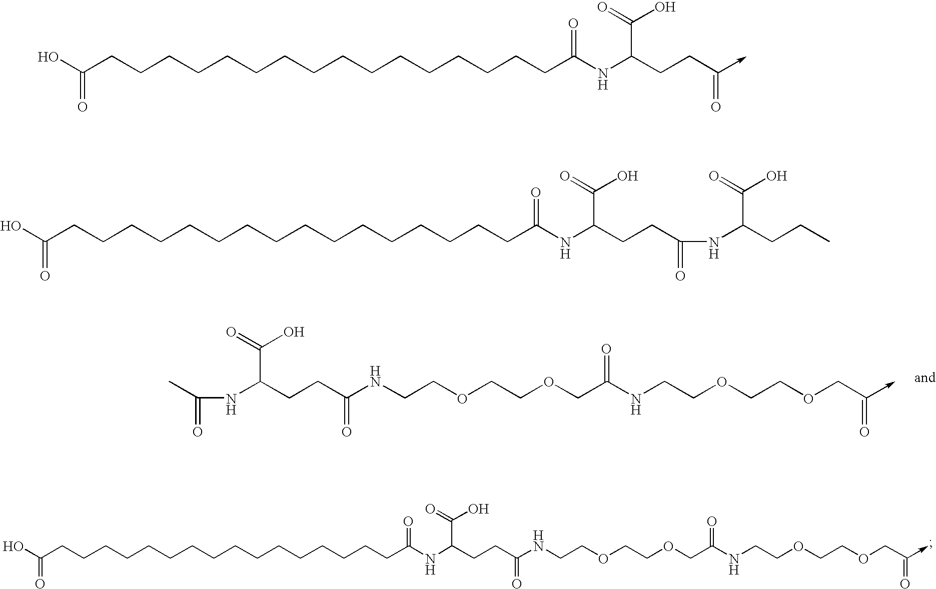

KCNTATCATQRLANFLVRSSNNFGPILPPTNVGSNTY (SEQ ID No 10), wherein the lysine residue in position 1 is linked to an albumin binding residue with a formula selected from the group consisting of:

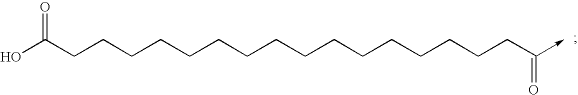

KCKTATCATQRLANFLVRSSNNFGPILPPTNVGSNTY (SEQ ID No 11), wherein the lysine residue in position 3 is linked to an albumin binding residue with a formula selected from the group consisting of:

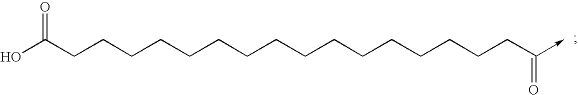

KCGTATCATQRLANFLARSSNNFGPILSPTNVGSNTY (SEQ ID No 12), wherein the glycine residue in position 3 is linked to an albumin binding residue combined with a linker with a formula selected from the group consisting of:

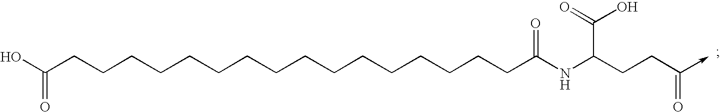

EERECNTATCATQRLANFLVHSSNNFGPILPPTNVGSNTY (SEQ ID No 13), wherein the glutamic acid residue in position 1 is linked to an albumin binding residue combined with a linker with a formula selected from the group consisting of:

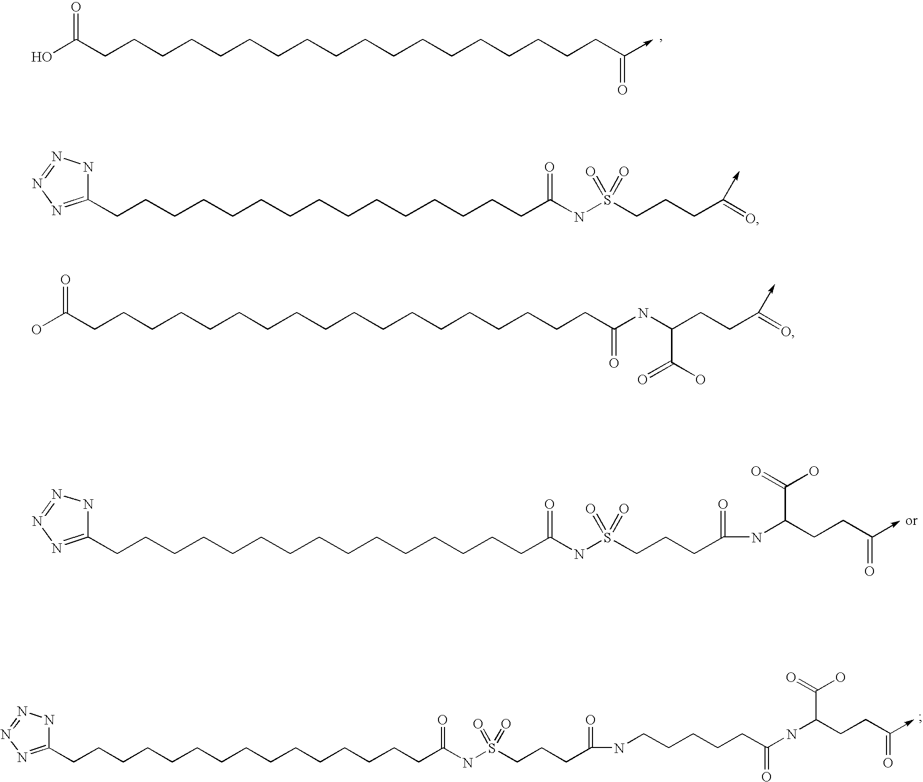

KCKTATCATQRLANFLVRSSQNLGPVLPPTNVGSNTY (SEQ ID No 14), wherein the lysine residue in position 3 is linked to an albumin binding residue combined with a linker with a formula selected from the group consisting of:

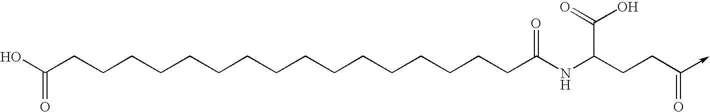

ECKTATCATQRLANFLVHSSNNFGPILPPTNVGSNTY (SEQ ID No 15), wherein the lysine residue in position 3 is linked to an albumin binding residue with a formula selected from the group consisting of:

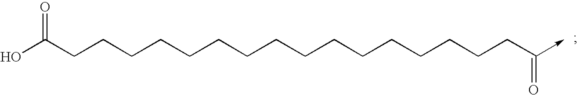

EECNTATCATQRLANFLVHSSNNFGPILPPTNVGSNTY (SEQ ID No 16), wherein the glutamic acid residue in position 1 is linked to an albumin binding residue with a formula selected from the group consisting of:

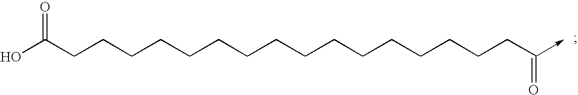

EERECQTATCATQRLANFLVRSSQNLGPVLPPTNVGSNTY (SEQ ID No 19) or EEEECQTATCATQRLANFLVRSSQNLGPVLPPTNVGSNTY (SEQ ID No 20), wherein the glutamic acid residue in position 1 is linked to an albumin binding residue with a formula:

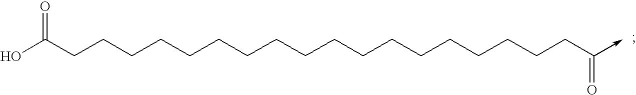

CKTATCATQRLANFLVHSSNNFGPILPPTNVGSNTY (SEQ ID No 21), wherein the lysine residue in position 2 is linked to an albumin binding residue combined with a linker with a formula selected from the group consisting of:

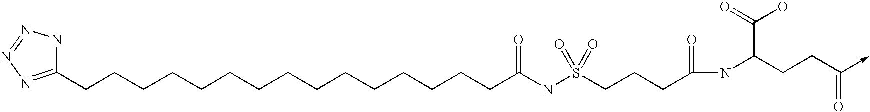

CNTATCATQRLANFLVHSSNNFGPILPPTNVGSNTY (SEQ ID No 22), wherein the cysteine residue in position 1 is linked to an albumin binding residue combined with a linker with a formula:

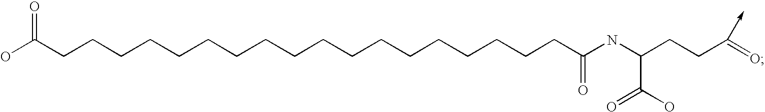

ECNTATCATQRLANFLVHSSNNFGPILPPTNVGSNTY (SEQ ID No 23), wherein the glutamic acid residue in position 1 is linked to an albumin binding residue combined with a linker with a formula:

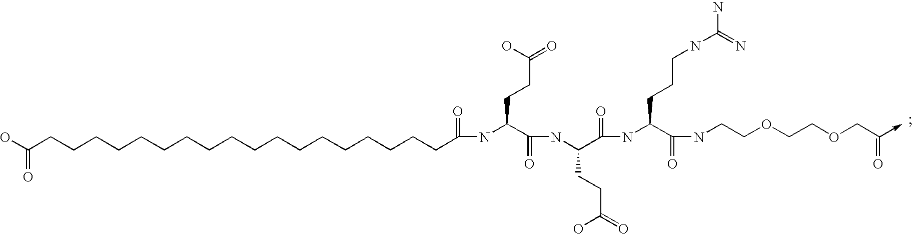

RCNTATCATQRLANFLVHSSNNFGPILPPTNVGSNTY (SEQ ID No 24), wherein the arginine residue in position 1 is linked to an albumin binding residue combined with a linker with a formula:

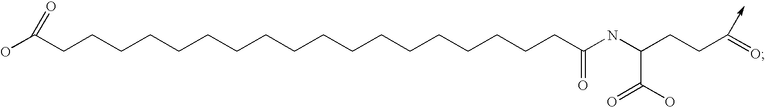

ERCNTATCATQRLANFLVHSSNNFGPILPPTNVGSNTY (SEQ ID No 25), wherein the glutamic acid residue in position 1 is linked to an albumin binding residue with a formula:

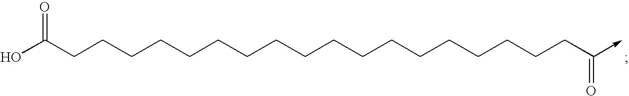

RRCNTATCATQRLANFLVHSSNNFGPILPPTNVGSNTY (SEQ ID No 26) and RKCNTATCATQRLANFLVHSSNNFGPILPPTNVGSNTY, (SEQ ID No 28) wherein the arginine residue in position 1 is linked to an albumin binding residue combined with a linker with a formula:

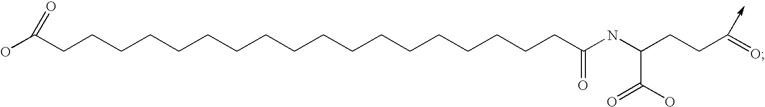

KKCNTATCATQRLANFLVHSSNNFGPILPPTNVGSNTY (SEQ ID No 27), wherein the lysine residue in position 1 is linked to an albumin binding residue combined with a linker with a formula:

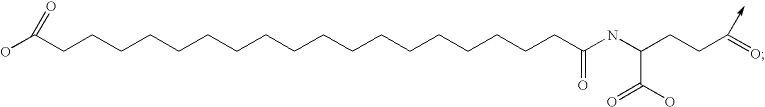

EKRKCNTATCATQRLANFLVHSSNNFGPILPPTNVGSNTY (SEQ ID No 29) and EKRCNTATCATQRLANFLVHSSNNFGPILPPTNVGSNTY (SEQ ID No 30) wherein the glutamic acid residue in position 1 is linked to an albumin binding residue with a formula:

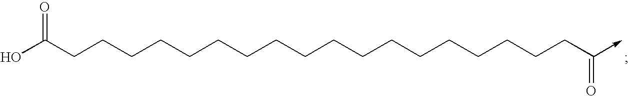

EERECKTATCATQRLANFLVRSSQNLGPVLPPTNVGSNTY (SEQ ID No 31), wherein the lysine residue in position 3 is linked to an albumin binding residue combined with a linker with a formula:

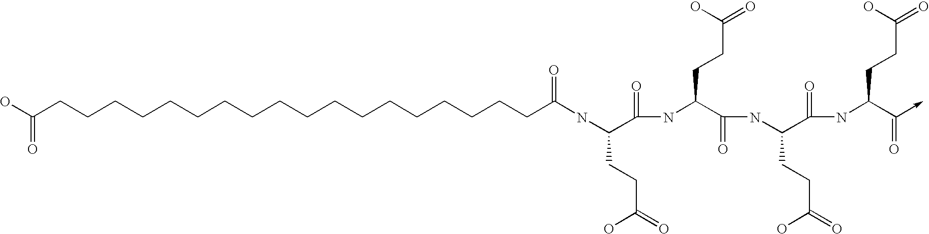

and
ECQTATCATQRLANFLVRSSQNLGPVLPPTNVGSNTY (SEQ ID No 32), wherein the glutamic acid residue in position 1 is linked to an albumin binding residue combined with a linker with a formula:

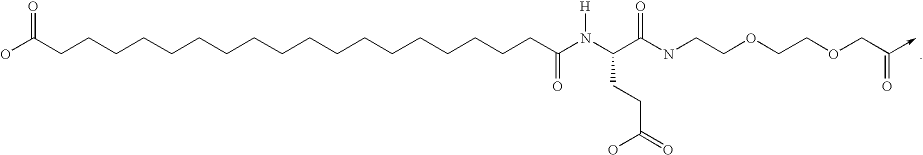

The invention relates in a further aspect to a method for increasing the time of action in a patient of human amylin or an analogue thereof, characterized by
a) modifying the amino acid residue in position 1 by optionally substituting with any natural amino acid, and by connecting said natural amino acid to a N-terminal extension consisting of 1-10 amino acids, and further
   a. linking said extension to an albumin binding residue optionally via a linker, or
   b. modifying an amino acid residue in position 2-37 by substitution with a lysine residue, and by linking said lysine residue to an albumin binding residue, optionally via a linker;
   or
b) modifying the amino acid residue in position 3 by substitution with a lysine residue and by linking said lysine residue to an albumin binding residue, optionally via a linker, and optionally modifying the amino acid in position 1 by substitution with a glutamine residue;
   or
c) modifying an amino acid residue in position 17, 21, 25 or 29 by substitution with a lysine residue and by linking said lysine residue to an albumin binding residue, optionally via a linker;
   or
d) modifying the amino acid residue in position 18 by substitution with an arginine residue, and further modifying an amino acid residue in position 1-17 or 19-37 by substitution with a lysine residue, and by linking to said lysine residue an albumin binding residue, optionally via a linker;
e) the amino acid in position 18 is arginine, the amino acids in position 25, position 28 and position 29 are proline, and an amino acid residue in position 1-17 or 19-37 has been substituted with a lysine residue, and wherein such a lysine residue is linked to an albumin binding residue, optionally via a linker.
   wherein the amino acid numbering conforms with the amino acid numbering in SEQ ID NO:17.

In another aspect, the invention relates to a method wherein the time of action in a patient of human amylin or an analogue thereof is increased to more than about 40 hours.

The production of peptides such as human amylin or analogues thereof is well known in the art. The peptides of the invention can thus be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The peptides may also be produced by a method which comprises culturing a host cell containing a DNA sequence encoding the polypeptide and capable of expressing the polypeptide in a suitable nutrient medium under conditions permitting the expression of the peptide. For peptides comprising non-natural amino acid residues, the recombinant cell should be modified such that the non-natural amino acids are incorporated into the peptide, for instance by use of tRNA mutants.

### Pharmaceutical Compositions

Pharmaceutical compositions containing a derivative according to the present invention may be prepared by conventional techniques, e.g. as described in Remington's Pharmaceutical Sciences, 1985 or in Remington: The Science and Practice of Pharmacy, 19^{th} edition, 1995.

One object of the present invention is to provide a pharmaceutical formulation comprising a peptide according to the present invention. In one aspect, the peptide is present in the formulation at a concentration of from about 0.1 mg/ml to about 25 mg/ml. In another aspect, the peptide is present in the formulation at a concentration of from about 1 mg/ml to about 10 mg/ml.

In another aspect, the formulation has a pH from 2.0 to 10.0.

In another aspect, the formulation has a pH from 2.0 to 7.0.

In yet another aspect, the formulation has a pH from 2.5-4.5.

In yet another aspect, the formulation has a pH from 2.5-4.0.

The formulation may further comprise a buffer system, preservative(s), isotonicity agent(s), chelating agent(s), stabilizers and/or surfactants. The use of such excipients in pharmaceutical compositions is well-known to the skilled person. For convenience reference is made to Remington: The Science and Practice of Pharmacy, 19^{th} edition, 1995.

In one aspect of the invention the pharmaceutical formulation is an aqueous formulation, i.e. formulation comprising water. Such formulation is typically a solution or a suspension. In a further aspect of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another aspect the pharmaceutical formulation is a freeze-dried formulation, whereto the physician or the patient adds solvents and/or diluents prior to use.

In another aspect the pharmaceutical formulation is a dried formulation (e.g. freeze-dried or spray-dried) ready for use without any prior dissolution.

In a further aspect of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative aspect of the invention.

In a further aspect of the invention the formulation further comprises a pharmaceutically acceptable preservative. In a further aspect of the invention the formulation further comprises an isotonic agent. In a further aspect of the invention the formulation further comprises a chelating agent.

In a further aspect of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject.

By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one aspect, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one aspect the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further aspect of the invention the amino acids or amino acid analogues are used in a concentration, which is sufficient to prevent or delay aggregation of the protein.

The pharmaceutical compositions may also comprise additional stabilizing agents, which further enhance stability of a therapeutically active polypeptide therein. The use of a stabilizing agents in pharmaceutical compositions is well-known to the skilled person. For convenience reference is made to Remington: The Science and Practice of Pharmacy, 19^{th} edition, 1995.

In a further aspect of the invention the formulation further comprises a surfactant. In a further aspect of the invention the formulation further comprises protease inhibitors. The use of a protease inhibitor is particular useful in pharmaceutical compositions comprising zymogens of proteases in order to inhibit autocatalysis.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a derivative of human amylin or an analogue thereof according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the derivative of human amylin or an analogue thereof increase bioavailability, increase solubility, decrease adverse effects, achieve chronotherapy well known to those skilled in the art, and increase patient compliance or any combination thereof.

Compositions of the current invention are useful in the formulation of solids, semisolids, powder and solutions for pulmonary administration of the derivative of human amylin or an analogue thereof, using, for example a metered dose inhaler, dry powder inhaler and a nebulizer, all being devices well known to those skilled in the art.

Compositions of the current invention are useful in the formulation of controlled, sustained, protracting, retarded, and slow release drug delivery systems.

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the derivative of human amylin or an analogue thereof in the form of a nasal or pulmonal spray. As a still further option, the pharmaceutical compositions containing the derivative of human amylin or an analogue thereof of the invention can also be adapted to transdermal administration, e.g. by needle-free injection or from a patch, optionally an iontophoretic patch, or transmucosal, e.g. buccal, administration.

The derivative of human amylin or an analogue thereof can be administered via the pulmonary route in a vehicle, as a solution, suspension or dry powder using any of known types of devices suitable for pulmonary drug delivery. Examples of these comprise of, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit. Rev Ther Drug Carr Sys 14(4) (1997) 395-453).

The term “stabilized formulation” refers to a formulation with increased physical stability, increased chemical stability or increased physical and chemical stability compared to an aqueous solution of the peptide.

The term “physical stability” of the protein formulation or a pharmaceutical formulation as used herein refers to the tendency of the protein not to form biologically inactive and/or insoluble aggregates of the protein as a result of exposure of the protein to thermomechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation or a pharmaceutical formulation as used herein refers to no chemical covalent changes in the protein structure which thereby do not lead to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

In one aspect of the invention the pharmaceutical formulation comprising the derivative of human amylin or an analogue thereof is stable for more than 6 weeks of usage and for more than 3 years of storage.

In another aspect of the invention the pharmaceutical formulation comprising the derivative of human amylin or an analogue thereof is stable for more than 4 weeks of usage and for more than 3 years of storage.

In a further aspect of the invention the pharmaceutical formulation comprising the derivative of human amylin or an analogue thereof is stable for more than 4 weeks of usage and for more than two years of storage.

In an even further aspect of the invention the pharmaceutical formulation comprising the derivative of human amylin or an analogue thereof is stable for more than 2 weeks of usage and for more than two years of storage.

In another aspect, the present invention relates to a derivative according to the invention for use as a medicament.

In one aspect, a derivative according to the invention is used for the preparation of a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, stroke, coronary heart disease and other cardiovascular disorders, inflammatory bowel syndrome, dyspepsia and gastric ulcers.

In another aspect, a derivative according to the invention is used as a medicament for delaying or preventing disease progression in type 2 diabetes.

In another aspect, a derivative according to the invention is used as a medicament for decreasing food intake, decreasing β-cell apoptosis, increasing β-cell function and β-cell mass, and/or for restoring glucose sensitivity to β-cells.

In one aspect of the invention, the derivative according to the invention is for use as a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers or for delaying or preventing disease progression in type 2 diabetes or for decreasing food intake, decreasing β-cell apoptosis, increasing β-cell function and β-cell mass, and/or for restoring glucose sensitivity to β-cells, is provided.

In a further aspect of the invention, a method for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers or for delaying or preventing disease progression in type 2 diabetes or for decreasing food intake, decreasing β-cell apoptosis, increasing β-cell function and β-cell mass, and/or for restoring glucose sensitivity to β-cells, the method comprising administering to a patient in need of such treatment an effective amount for such treatment of a derivative according to the invention, is provided.

The treatment with a derivative according to the present invention may also be combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, gastrin and gastrin analogs.

It should be understood that any suitable combination of the derivatives according to the invention with one or more of the above-mentioned compounds and optionally one or more further pharmacologically active substances are considered to be within the scope of the present invention.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### Assays

Pharmacological Assay (I)—Experimental Protocol for Efficacy Testing on Appetite with an Amylin Derivative, Using an Ad Libitum Fed Rat Model.

Sprague Dawley (SD) rats from Taconic Europe, Denmark are used for the experiments. The rats have a body weight 200-250 g at the start of experiment. The rats arrive at least 10-14 days before start of experiment to allow acclimatization to experimental settings. During this period the animals are handled at least 2 times. After arrival rats are housed individually for one week in a reversed light/dark phase (meaning that lights are off during daytime and on during nighttime) for two weeks. Since rats are normally active and eat their major part of their daily food intake during the dark period, rats are dosed in the morning right before lights are turned off. This set-up results in the lowest data variation and highest test sensitivity. The experiment is conducted in the rats' home cages and rats have free access to food and water throughout the acclimatization period and the experiment period. Each dose of derivative is tested in a group of 5-8 rats. A vehicle group of 6-8 rats is included in each set of testing. Rats are dosed once according to body weight with a 0.01-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually continuously by on-line registration or manually every hour for 7 hours, and then after 24 h and sometimes 48 h. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

Pharmacological Assay (II)—Experimental Protocol for Efficacy Testing on Appetite with an Amylin Derivative, Using a Schedule Fed Rat Model.

TAC:SPRD @mol rats or Wistar rats from M&B Breeding and Research Centre A/S, Denmark are used for the experiments. The rats have a body weight 200-250 g at the start of experiment. The rats arrive at least 10-14 days before start of experiment with a body weight of 180-200 g. Each dose of derivative is tested in a group of 6-8 rats. A vehicle group of 6-8 rats is included in each set of testing.

When the animals arrive they are housed individually. At least 7 days prior to onset of study the will start training to a feeding schedule allowing them to have free access to food and water in a scheduled time period between 3 and 7 h. In the remaining period of the day, the rats will not have access to food, but only water. Within a week rats will eat the complete daily ration in the set schedule. Since rats normally initiate food intake when light is removed, and eat the major part of their daily food intake during the night, this set up results allow for monitoring of food intake during day time and will typically mean less variation in the vehicle group compared to an ad libitum fed rat. During the acclimatization period of 10-14 days, the rats have free access to food and water. During this period the animals are handled at least 3 times. The experiment is conducted in the rats' home cages. Immediately before dosing the rats are randomised to the various treatment groups (n=6-8) by body weight. They are dosed according to body weight at between 15 to 30 min prior to given access to food with a 0.01-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually continuously by on-line registration or manually every hour during the schedule. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

### Luciferase Assay (II)

1. Amylin Assay Outline

It has previously been published (Poyner D R et al 2002, Pharmacological Reviews 54(2) 233-246) that activation of Amylin receptors (coexpression of Calcitonin receptor and receptor activity modifying peptides RAMPs) by Amylin leads to an increase in the intracellular concentration of cAMP. Consequently, transcription is activated at promotors containing multiple copies of the cAMP response element (CRE). It is thus possible to measure Amylin activity by use of a CRE luciferase reporter gene introduced into BHK cells also expressing an Amylin receptor.

2. Construction of an Amylin 3(a)/CRE-luc Cell Line

A BHK570 cell line stably transfected with the human calcitonin receptor (CTa) and a CRE-responsive luciferase reportergene. The cell line was further transfected with RAMP-3, using standard methods. This turns the Calcitonin receptor into an Amylin 3(a) receptor. Methotrexate, Neomycin, and Hygromycin are selection markers for luciferase, the Calcitonin receptor, and RAMP-3, respectively.

3. Amylin Luciferase Assay

To perform activity assays, BHK Amylin 3(a)//CRE-luc cells were seeded in white 96 well culture plates at a density of about 20.000 cells/well. The cells were in 100 μl growth medium (DMEM with 10% FBS, 1% Pen/Strep, 1 mM Na-pyruvate, 250 nM Methotrexate, 500 μg/ml Neomycin, and 400 μg/ml Hygromycin). After incubation overnight at 37° C. and 5% CO₂, the growth medium was replaced by 50 μl/well assay medium (DMEM (without phenol red), Glumamax™, 10% FBS, and 10 mM Hepes, pH 7,4). Further, 50 μl/well of standard or sample in assay buffer were added. After 4 hours incubation at 37° C. and 5% CO₂, the assay medium with standard or sample were removed and replaced by 100 μl/well PBS. Further, 100 μl/well LucLite™ was added. The plates were sealed and incubated at room temperature for 30 minutes. Finally, luminescence was measured on a TopCounter (Packard) in SPC (single photon counting) mode.

### General Introduction to ThT Fibrillation Assays for the Assessment of Physical Stability of Protein Formulations

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. Biochem. 177, 244-249; LeVine (1999) Methods. Enzymol. 309, 274-284].

The time course for fibril formation can be described by a sigmoidal curve with the following expression [Nielsen et al. (2001) Biochemistry 40, 6036-6046]:

$$F = {f}_{i} + {m}_{i} t + \frac{{f}_{f} + {m}_{f} t}{1 + {e}^{- [ ( t - {t}_{0} ) / τ ]}} \qquad Eq . ( 1 )$$

Here, F is the ThT fluorescence at the time t. The constant t₀ is the time needed to reach 50% of maximum fluorescence. The two important parameters describing fibril formation are the lag-time calculated by t₀−2τ and the apparent rate constant kₐₚₚ=1/τ.

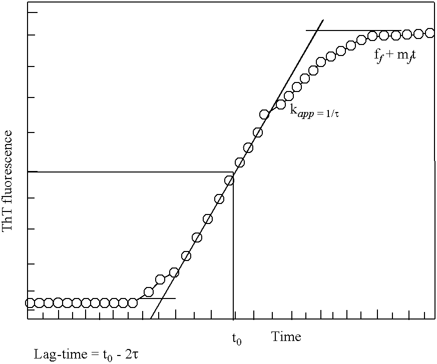

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

### Sample Preparation

Samples were prepared freshly before each assay. Each sample composition is described in each example. The pH of the sample was adjusted to the desired value using appropriate amounts of concentrated NaOH and HClO₄ or HCl. Thioflavin T was added to the samples from a stock solution in H₂O to a final concentration of 10 μM. Sample aliquots of 200 μl were placed in a 96 well microtiter plate (Packard OptiPlate™-96, white polystyrene). Usually, four or eight replica of each sample (corresponding to one test condition) were placed in one column of wells. The plate was sealed with Scotch Pad (Qiagen).

### Incubation and Fluorescence Measurement

Incubation at given temperature, shaking and measurement of the ThT fluorescence emission were done in a Fluoroskan Ascent FL fluorescence platereader or Varioskan platereader (Thermo Labsystems). The temperature was adjusted to 37° C. The orbital shaking was adjusted to 960 rpm with an amplitude of 1 mm in all the presented data. Fluorescence measurement was done using excitation through a 444 nm filter and measurement of emission through a 485 nm filter.

Each run was initiated by incubating the plate at the assay temperature for 10 min. The plate was measured every 20 minutes for a desired period of time. Between each measurement, the plate was shaken and heated as described.

### Data Handling

The measurement points were saved in Microsoft Excel format for further processing and curve drawing and fitting was performed using GraphPad Prism. The background emission from ThT in the absence of fibrils was negligible. The data points are typically a mean of four or eight samples and shown with standard deviation error bars. Only data obtained in the same experiment (i.e. samples on the same plate) are presented in the same graph ensuring a relative measure of fibrillation between experiments.

The data set may be fitted to Eq. (1). However, since full sigmodial curves in this case are not always achieved during the measurement time, the degree of fibrillation is expressed as ThT fluorescence tabulated as the mean of the samples and shown with the standard deviation at various time points.

### EXAMPLES

The compounds of examples 1-51 were prepared according to the below-mentioned peptide Synthesis:

One method of peptide synthesis was by Fmoc chemistry on a microwave-based Liberty peptide synthesizer (CEM Corp., North Carolina). The resin was Tentagel S RAM with a loading of 0.25 mmol/g. The coupling chemistry was DIC/HOAt in NMP using amino acid solutions of 0.3 M in NMP and a molar excess of 6-8 fold. Coupling conditions was 5 minutes at up to 70° C. Deprotection was with 5% piperidine in NMP at up to 70° C. The protected amino acids used were standard Fmoc-amino acids (supplied from e.g. Anaspec or Novabiochem) dissolved at 0.3 M in NMP containing 0.3 M HOAt.

Another method of peptide synthesis was on an Applied Biosystems 433 peptide synthesizer in 0.25 mmol or 1.0 mmol scale using the manufacturer supplied FastMoc UV protocols which employ HBTU (2-(1H-Benzotriazol-1-yl-)-1,1,3,3 tetramethyluronium hexafluorophosphate) or HATU (O-(7-azabenzotriazol-1-yl)-1,1,3,3-tetramethyluronium hexafluorophosphate) mediated couplings in NMP, and UV monitoring of the deprotection of the Fmoc protection group. The starting resin used for the synthesis of the peptide amides was Rink-Amide resin. The protected amino acid derivatives used were standard Fmocamino acids (supplied from e.g. Anaspec, or Novabiochem) supplied in preweighed cartridges suitable for the ABI433A synthesizer.

When a chemical modification of a lysine side chain was desired, the lysine was incorporated as Lys(Mtt) and the N-terminal amino acid was either incorporated into the sequence as a Boc-amino acid or, if the N-terminal amino acid was incorporated as an Fmoc-amino acid, the Fmoc group was removed and the N-terminal was protected by treatment with 6 equivalents of Boc-carbonate and 6 equivalents of DIPEA in NMP for 30 minutes. The resin was washed with NMP and DCM and the Mtt group was removed by suspending the resin in neat hexafluoroisopropanol for 20 minutes followed by washing with DCM and NMP. The chemical modification of the lysine was performed by adding one or more of the building blocks listed below by the same methods as used for the peptide synthesis, i.e. by one or more automated steps on the Liberty or the ABI 433 or by one or more manual coupling steps at room temperature. After synthesis the resin was washed with DCM and dried, and the peptide was cleaved from the resin by a 2 hour treatment with TFA/TIPS/water (92.5/5/2.5) followed by precipitation with 4 volumes of diethylether. After further washing with diethylether and drying, the peptide was redissolved in water at 1-2 mg/ml, pH adjusted to about 4.5, and the disulfide bridge formed by treatment with 1.1 eq. of [Pt(IV) ethylenediamine₂Cl₂]Cl₂ overnight. Alternatively, the disulfide bridge was formed on the resin by treatment with 10 equivalents of iodine in NMP for 1 hour. In this case the crude peptide was purified directly after cleavage and diethylether precipitation.
Purification: The crude peptide was purified by semipreparative HPLC on a 20 mm×250 mm column packed with either 5μ or 7μ C-18 silica. Peptide solutions were pumped onto the HPLC column and precipitated peptides were dissolved in 5 ml 50% acetic acid H₂O and diluted to 20 ml with H₂O and injected on the column which then was eluted with a gradient of 40-60% CH₃CN in 0.1% TFA 10 ml/min during 50 min at 40° C. The peptide containing fractions were collected. The purified peptide was lyophilized after dilution of the eluate with water.
For analysis of HPLC-fractions and final product RP-HPLC analysis was performed using UV detection at 214 nm and e.g. a Vydac 218TP54 4.6 mm×250 mm 5μ C-18 silica column (The Separations Group, Hesperia, USA) and eluted at e.g. 1 ml/min at 42° C. Most often one of four different elution conditions was used:
A1: Equilibration of the column with a buffer consisting of 0.1M (NH₄)₂SO₄, which was adjusted to pH 2.5 with concentrated H₂SO₄ and elution by a gradient of 0% to 60% CH₃CN in the same buffer during 50 min.
B1: Equilibration of the column with 0.1% TFA/H₂O and elution by a gradient of 0% CH₃CN/0.1% TFA/H₂O to 60% CH₃CN/0.1% TFA/H₂O during 50 min.
B6: Equilibration of the column with 0.1% TFA/H₂O and elution by a gradient of 0% CH₃CN/0.1% TFA/H₂O to 90% CH₃CN/0.1% TFA/H₂O during 50 min.
Alternatively the RP-HPLC analysis was performed using UV detection at 214 nm and a Symmetry300, 3.6 mm×150 mm, 3.5μ C-18 silica column (Waters) which was eluted at 1 ml/min at 42° C.
B4: Equilibration of the column with 0.05% TFA/H₂O and elution by a gradient of 5% CH₃CN/0.05% TFA/H₂O to 95% CH₃CN/0.05% TFA/H₂O during 15 min.
The identity of the peptide was confirmed by MALDI-MS on a Bruker Microflex.

### Abbreviations Used:

HBTU: 2-(1H-Benzotriazol-1-yl-)-1,1,3,3 tetramethyluronium hexafluorophosphate
Fmoc: 9H-fluoren-9-ylmethoxycarbonyl
Boc: tert butyloxycarbonyl
Mtt: 4-methyltrityl
DCM: dichloromethane
TIPS: triisopropylsilane
TFA: trifluoroacetic acid

### NMP: 1-Methyl-pyrrolidin-2-one

HOAt: 1-Hydroxy-7-azabenzotriazole

### DIC: Diisopropylcarbodiimide

Trt: triphenylmethyl
The compounds of examples 1-51 were prepared according to the procedure above with the following building blocks and the following sequences:

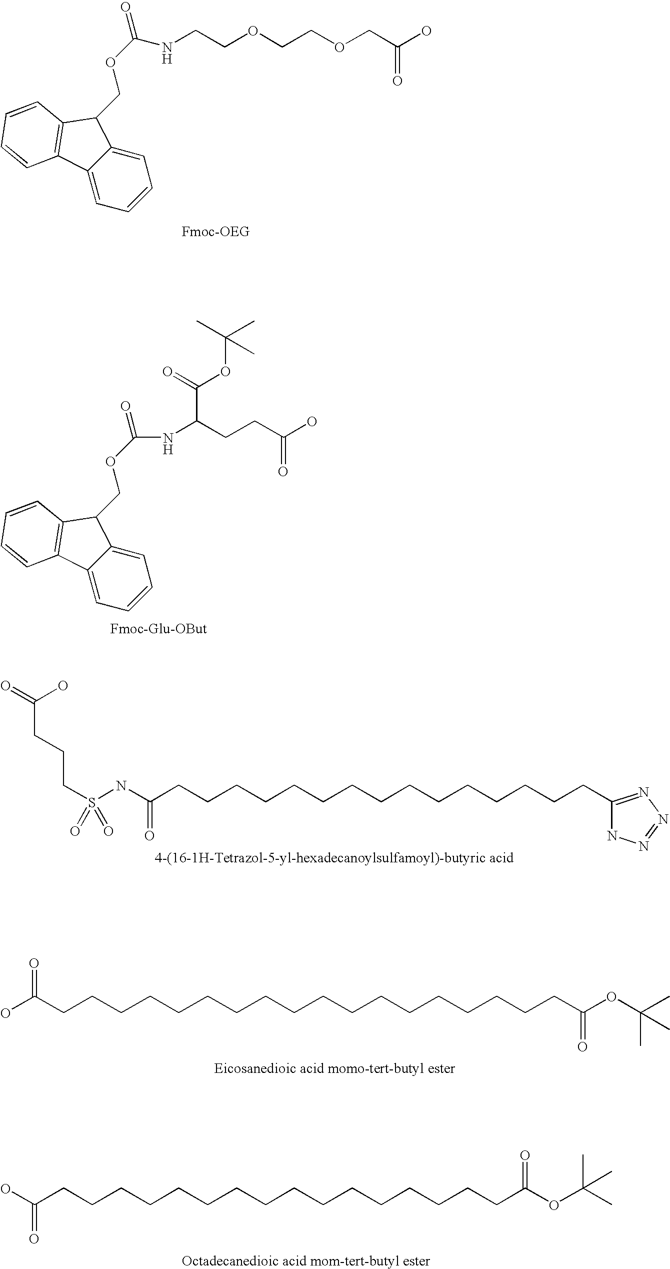

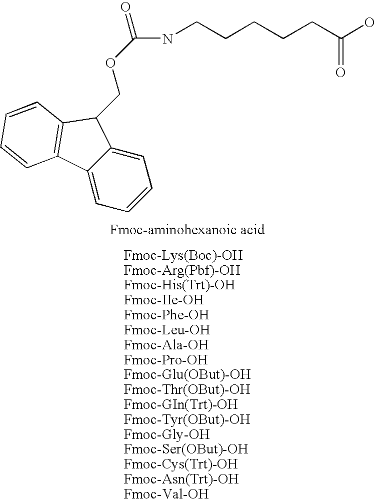

| | | Modifications |
|---|---|---|
| Seq | | compared to seq |
| # | Peptide sequence | ID no. 1 |
| 01 | KCNTATCATQRLANFLVHSSNNFGPILPPTNVGSNTY | (pramlintide) |
| 02 | KCKTATCATQRLANFLVHSSNNFGPILPPTNVGSNTY | K3 |
| 03 | KCNTATCATQRLANFLVHSSNNFGPILPKTNVGSNTY | K29 |
| 04 | KCNTATCATQRLANFLVHSSKNFGPILPPTNVGSNTY | K21 |
| 05 | KCNTATCATQRLANFLKHSSNNFGPILPPTNVGSNTY | K17 |
| 06 | KCNTATCATQRLANFLVHSSNNFGKILPPTNVGSNTY | K25 |
| 07 | KCNTATCATQRLANFLVRSSNNFGKILPPTNVGSNTY | R18, K25 |
| 08 | CNTATCATQRLANFLVRSSNNLGPVLPPTNVGSNTY | desK1, R18, L23, V |
| | | 26 |
| 09 | KCKTATCATQRLANFLVPSSNNFGPILPPTNVGSNTY | K3, P18 |
| 10 | KCNTATCATQRLANFLVRSSNNFGPILPPTNVGSNTY | R18 |
| 11 | KCKTATCATQRLANFLVRSSNNFGPILPPTNVGSNTY | K3, R18 |
| 12 | KCGTATCATQRLANFLARSSNNFGPILSPTNVGSNTY | G3, A17, R18, S28 |
| 13 | EERECNTATCATQRLANFLVHSSNNFGPILPPTNVGSNTY | EER-E1 |
| 14 | KCKTATCATQRLANFLVRSSQNLGPVLPPTNVGSNTY | K3, R18, Q21, L23, |
| | | V26 |
| 15 | ECKTATCATQRLANFLVESSNNFGPILPPTNVGSNTY | E1, K3, E18 |
| 16 | EECNTATCATQRLANFLVHSSNNFGPILPPTNVGSNTY | E-E1 |
| 17 | KCNTATCATQRLANFLVHSSNNFGAILSSTNVGSNTY | A25, S28, S29 |
| | | (Human amylin) |
| 18 | EERECQTATCATQRLANFLVRSSQNLGPVLPPTNVGSNTY | EER- |
| | | E1, Q3, R18, Q21, L |
| | | 23, V26 |
| 19 | EEEECQTATCATQRLANFLVRSSQNLGPVLPPTNVGSNTY | EEE- |
| | | E1, Q3, R18, Q21, L |
| | | 23, V26 |
| 20 | CKTATCATQRLANFLVHSSNNFGPILPPTNVGSNTY | K3 desK1 |
| 21 | CNTATCATQRLANFLVHSSNNFGPILPPTNVGSNTY | desK1 |
| 22 | ECNTATCATQRLANFLVHSSNNFGPILPPTNVGSNTY | E1 |
| 23 | RCNTATCATQRLANFLVHSSNNFGPILPPTNVGSNTY | R1 |
| 24 | ERCNTATCATQRLANFLVHSSNNFGPILPPTNVGSNTY | E-R1 |
| 25 | RRCNTATCATQRLANFLVHSSNNFGPILPPTNVGSNTY | R-R1 |
| 26 | KKCNTATCATQRLANFLVHSSNNFGPILPPTNVGSNTY | K |
| 27 | RKCNTATCATQRLANFLVHSSNNFGPILPPTNVGSNTY | R |
| 28 | EKRKCNTATCATQRLANFLVHSSNNFGPILPPTNVGSNTY | EKR |
| 29 | EKRCNTATCATQRLANFLVHSSNNFGPILPPTNVGSNTY | EK-R1 |
| 30 | EERECKTATCATQRLANFLVRSSQNLGPVLPPTNVGSNTY | EER- |
| | | E1, K3, R18, Q21, L |
| | | 23, V26 |
| 31 | ECQTATCATQRLANFLVRSSQNLGPVLPPTNVGSNTY | E1, Q3, R18, Q21, L |
| | | 23, V26 |

### Example 1

### N-alpha-(17-carboxyheptanoyl)-Glu-[Glu1]-pramlintide (1-37)

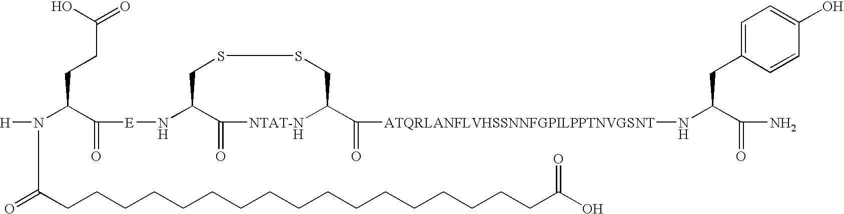

### Example 2

### N-epsilon3-(17-carboxyheptadecanoyl)-[Lys3]-pramlintide (1-37)

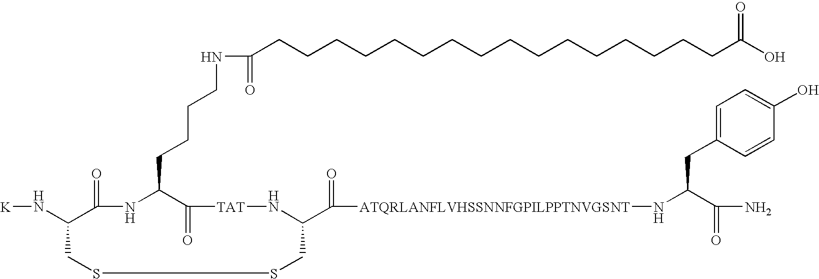

### Example 3

### N-epsilon3-(2-{2-[2-(2-{2-[2-(17-carboxyheptadecanoylamino) ethoxy]ethoxy}acetylamino)ethoxy]ethoxy}acetyl)-[Lys3]-pramlintide (1-37)

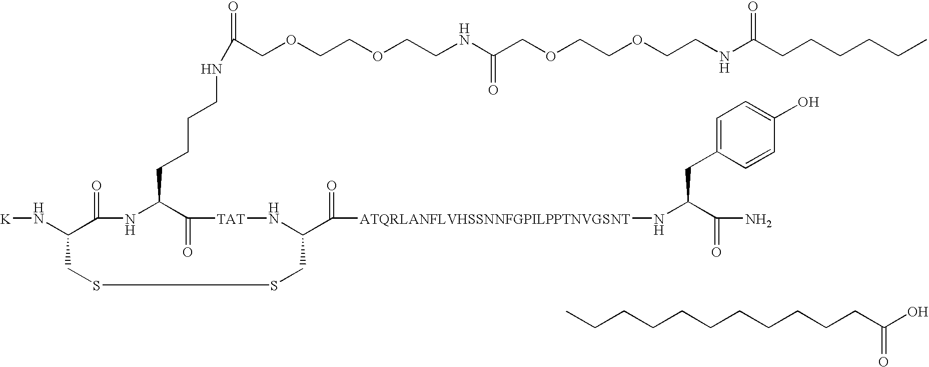

### Example 4

### N-epsilon29-(19-carboxynonadecanoyl)-[Lys29]-pramlintide (1-37)

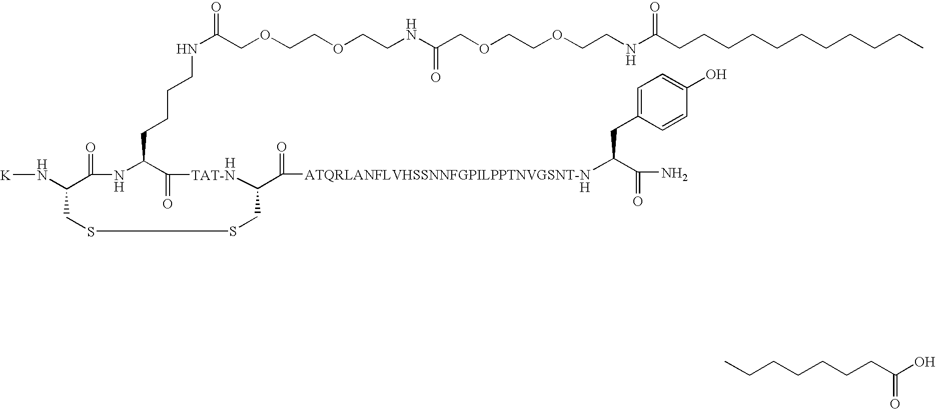

### Example 5

### N-epsilon29-(17-carboxyheptadecanoyl)-[Lys29]-pramlintide (1-37)

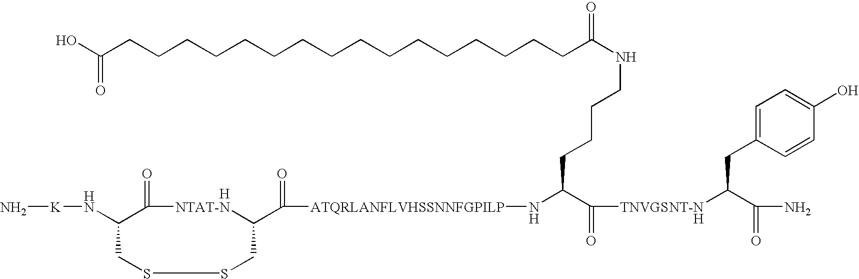

### Example 6

### N-epsilon29-(2-{2-[2-(2-{2-[2-(19-carboxynonadecanoylamino) ethoxy]ethoxy}acetylamino)ethoxy]ethoxy}acetyl)-[Lys29]-pramlintide (1-37)

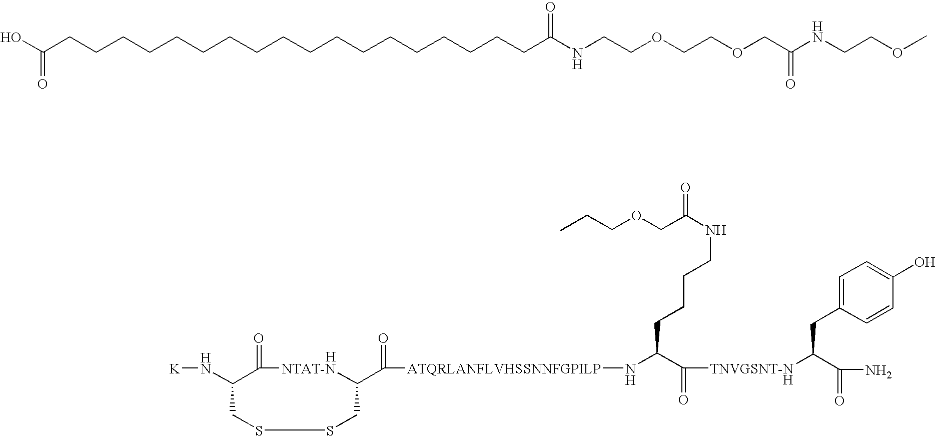

### Example 7

### N-epsilon2′-(2-{2-[2-(2-{2-[2-(19-carboxynonadecanoylamino) ethoxy]ethoxy}acetylamino)ethoxy]ethoxy}acetyl)-[Lys21]-pramlintide (1-37)

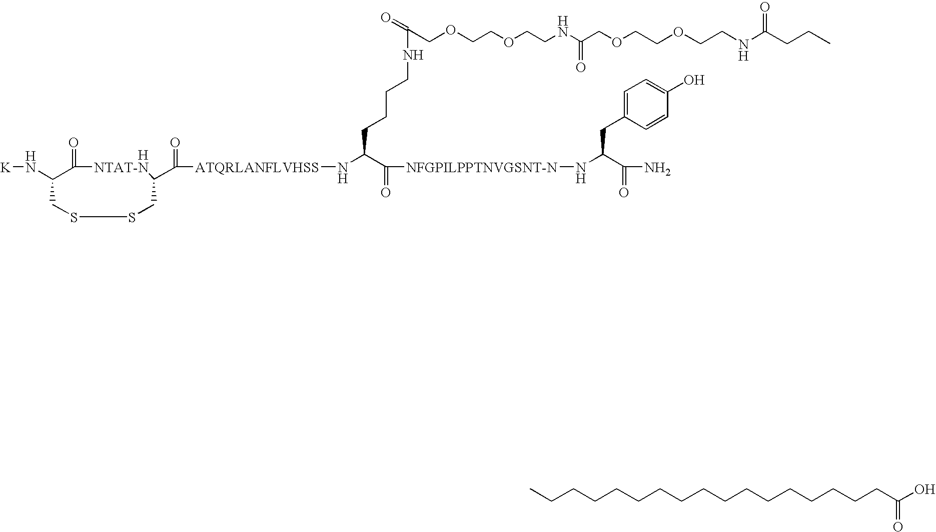

### Example 8

### N-epsilon17-(2-{2-[2-(2-{2-[2-(19-carboxynonadecanoylamino)ethoxy]ethoxy}acetylamino)ethoxy]ethoxy}acetyl)-[Lys17]-pramlintide (1-37)

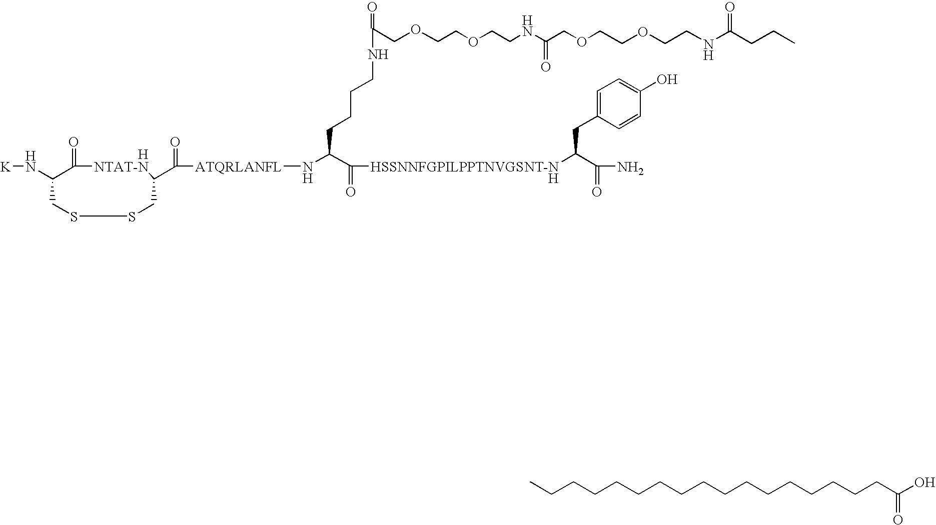

### Example 9

### N-epsilon25-(2-{2-[2-(2-{2-[2-(17-carboxyheptadecanoylamino) ethoxy]ethoxy}acetylamino)ethoxy]ethoxy}acetyl)-[Arg18,Lys25]-pramlintide (1-37)

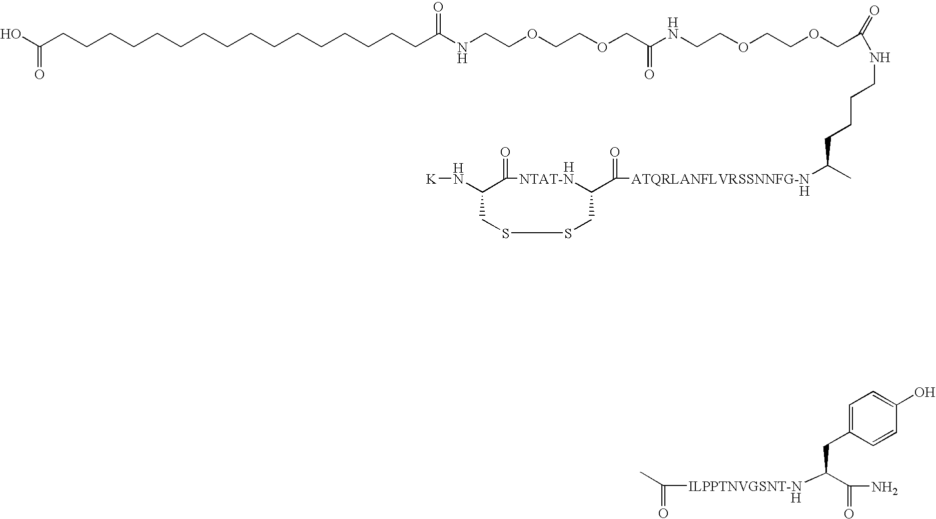

### Example 10

### N-epsilon25-(2-{2-[2-(2-{2-[2-(19-carboxynonadecanoylamino) ethoxy]ethoxy}acetylamino)ethoxy]ethoxy}acetyl)-[Lys25]-pramlintide (1-37)

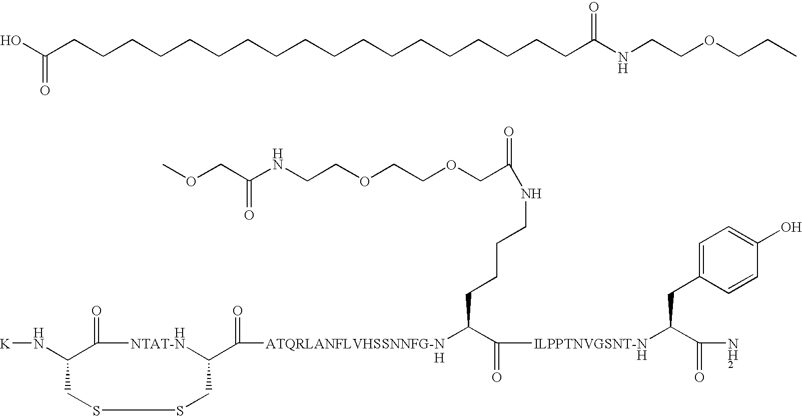

### Example 11

### N-epsilon3-[2-(2-{2-[2-(2-{2-[(S)-4-carboxy-4-(17-carboxyheptadecanoylamino)butyrylamino]ethoxy}ethoxy)acetylamino]ethoxy}ethoxy)acetyl]-[Lys3]-pramlintide (1-37)

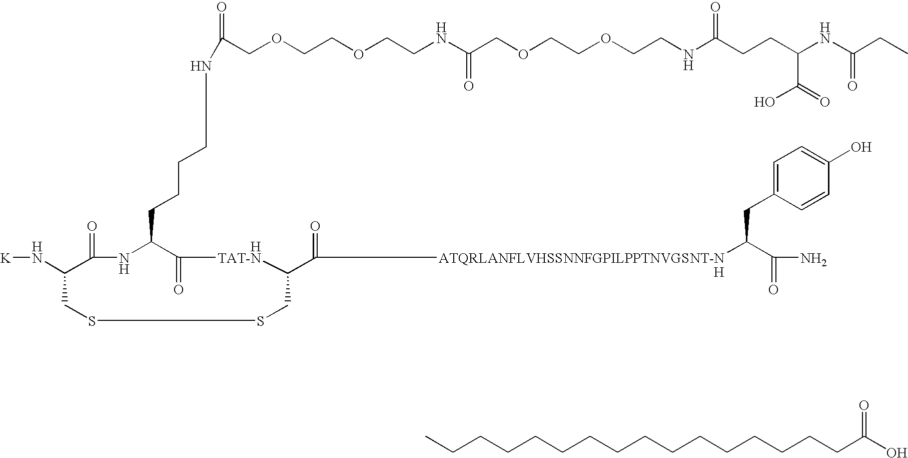

### Example 12

### N-epsilon3-[2-(2-{2-[2-(2-{2[(S)-4-carboxy-4-(19-carboxynonadecanoylamino) butyrylamino]ethoxy}ethoxy)acetylamino]ethoxy}ethoxy)acetyl]-[Lys3]-pramlintide (1-37)

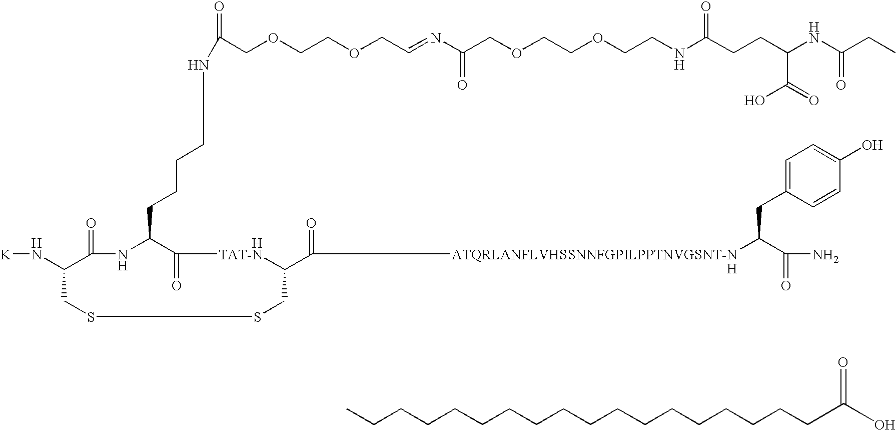

### Example 13

### N-epsilon3-(2-{2-[2-(2-{2-[2-(19-carboxynonadecanoylamino) ethoxy]ethoxy}acetylamino)ethoxy]ethoxy}acetyl)-[Lys3]-pramlintide (1-37)

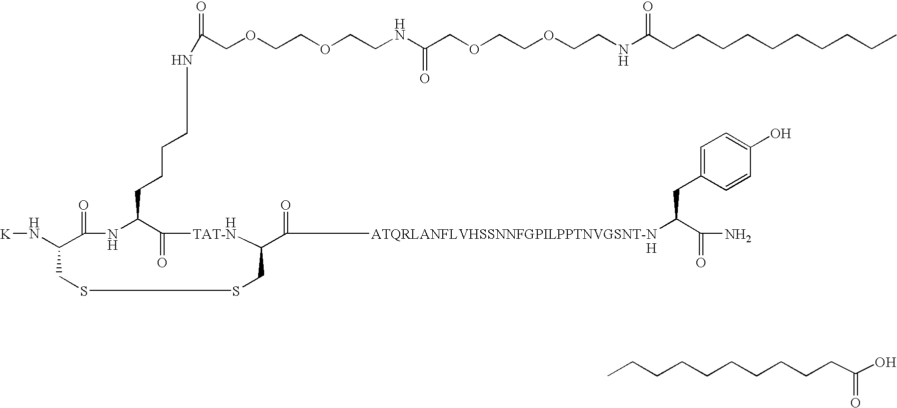

### Example 14

### N-epsilon3-(19-carboxynonadecanoyl)-[Lys3]-pramlintide (1-37)

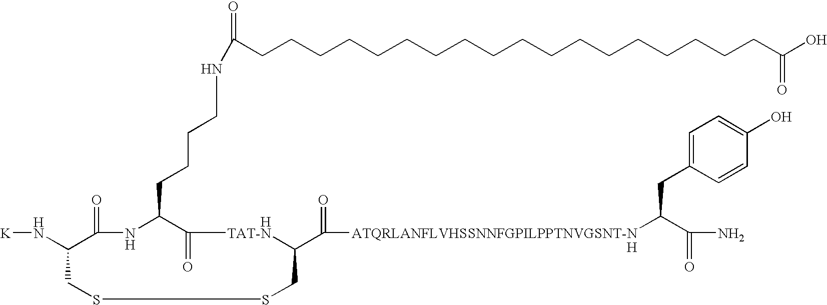

### Example 16

### N-alpha-[(S)-4-carboxy-4-(17-carboxyheptadecanoylamino)butyryl]-[Arg18,Leu23,Val26]-pramlintide (2-37)

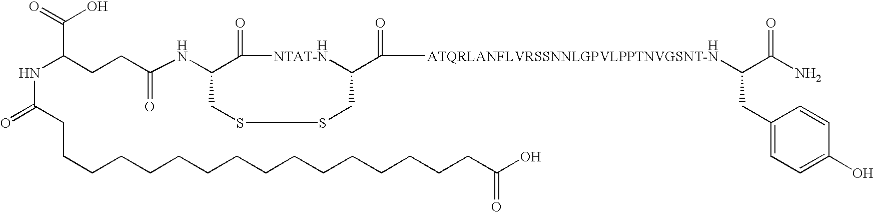

### Example 17

### N-alpha-(2-{2-[2-(2-{2-[2-((S)-4-Carboxy-4-{4-carboxy-4-[4-carboxy-4-(17-carboxyheptadecanoylamino)butyrylamino]butyrylamino}butyrylamino) ethoxy}ethoxy]acetylamino)ethoxy}ethoxy]acetyl)-[Arg18,Leu23,Val26]-pramlintide (2-37)

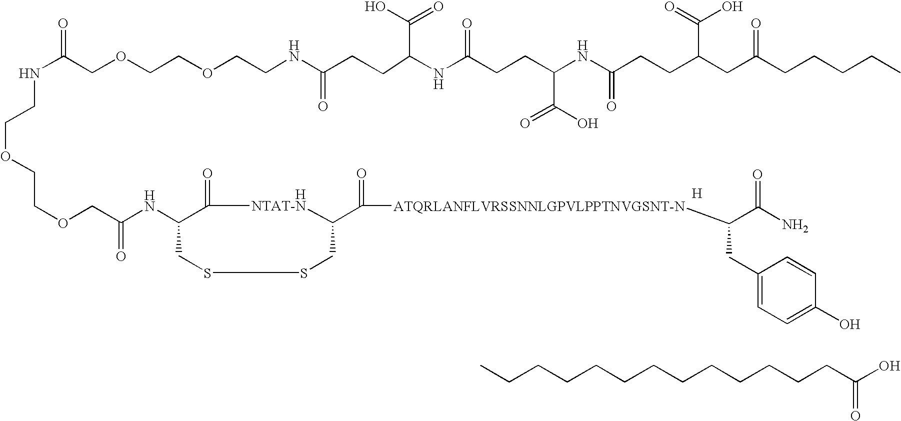

### Example 18

### N-epsilon3-[(S)-4-carboxy-4-(17-carboxyheptadecanoylamino)butyryl]-[Lys3,Pro18]-pramlintide (1-37)

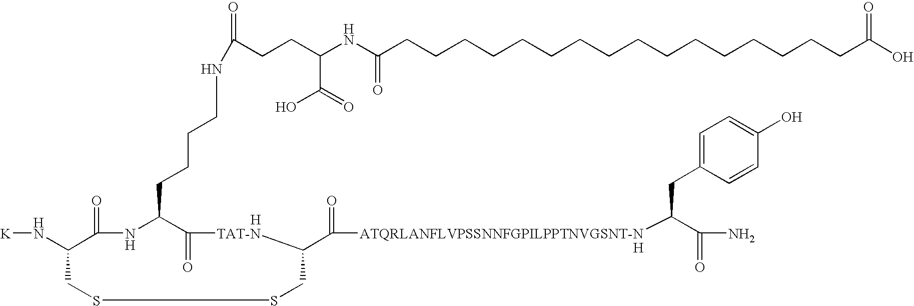

### Example 19

### N-epsilon3-[2-(2-{2-[2-(2-{2-[(S)-4-carboxy-4-(17-carboxyheptadecanoylamino)butyrylamino]ethoxy}ethoxy)acetylamino]ethoxy}ethoxy)acetyl]-[Lys3,Pro18]-pramlintide (1-37)

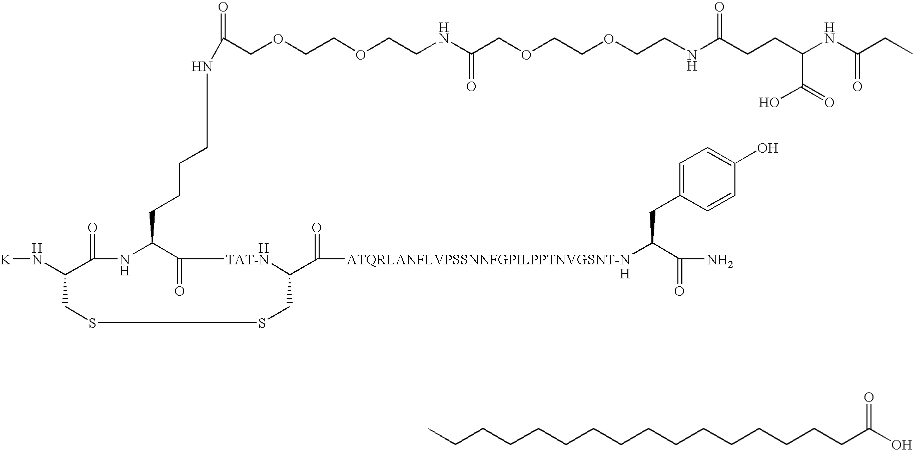

### Example 20

### N-epsilon3-(2-{2-[2-(2-{2-[2-((S)-4-carboxy-4-{4-carboxy-4-[4-carboxy-4-(17-carboxyheptadecanoylamino)butyrylamino]butyrylamino}butyrylamino) ethoxy}ethoxy]acetylamino)ethoxy}ethoxy]acetyl)-[Lys3,Pro18]-pramlintide (1-37)

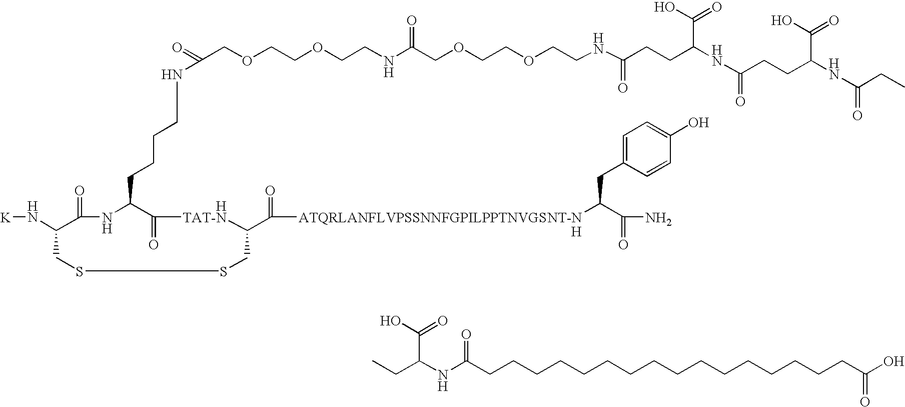

### Example 21

### N-alpha-(17-carboxyheptanoyl)-[Arg18]-pramlintide (1-37)

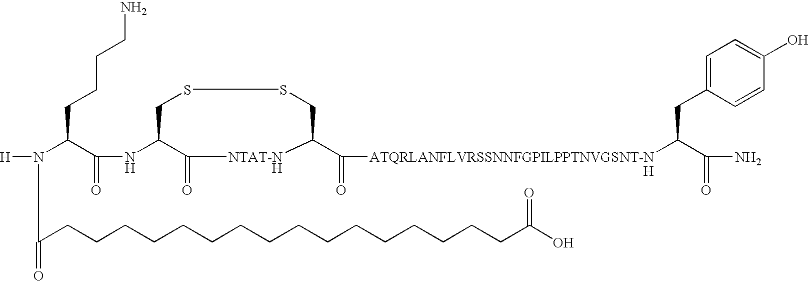

### Example 22

### N-epsilon3-(17-carboxyheptanoyl)-[Lys3,Arg18]-pramlintide (1-37)

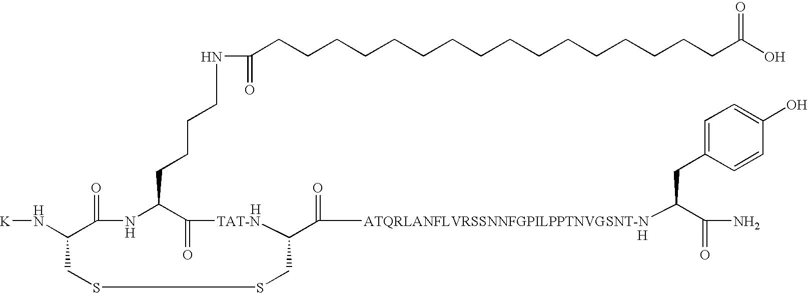

### Example 23

### N-epsilon1-[(S)-4-carboxy-4-(17-carboxyheptadecanoylamino)butyryl]-[Gly3,Ala17,Arg18,Ser28]-pramlintide (1-37)

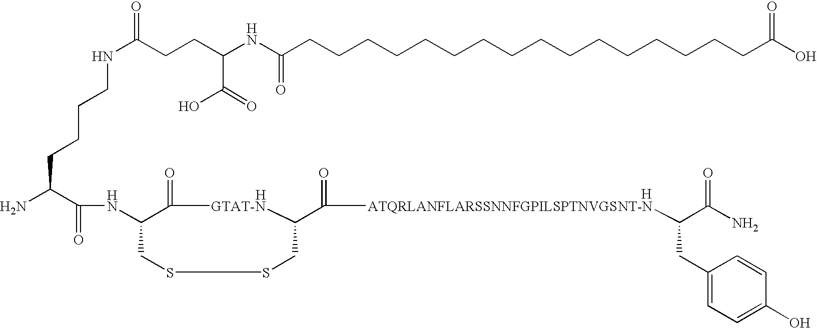

### Example 24

### N-alpha-(17-carboxyheptanoyl)-[Arg18,Leu23,Val26]-pramlintide (2-37)

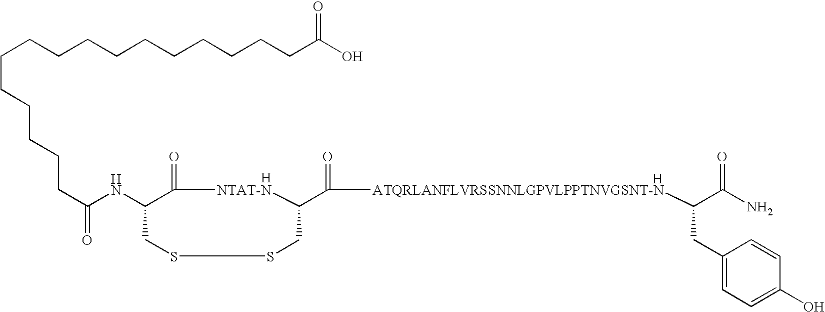

### Example 25

### N-alpha-(19-carboxynonadecanoyl)-Glu-Glu-Arg-[Glu1]-pramlintide (1-37)

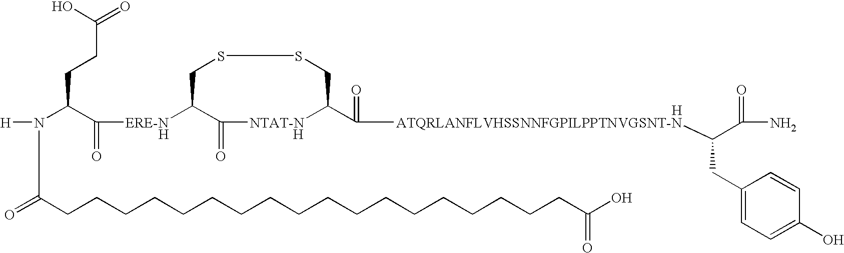

### Example 26

### N-epsilon25-[2-(2-{2-[2-(2-{2-[(S)-4-carboxy-4-((S)-4-carboxy-4-{(S)-4-carboxy-4-[(S)-4-carboxy-4-(17-carboxyheptadecanoylamino)butyrylamino]butyrylamino}butyrylamino)butyrylamino]ethoxy}ethoxy)acetylamino]ethoxy}ethoxy)acetyl]-[Lys25]-pramlintide (1-37)

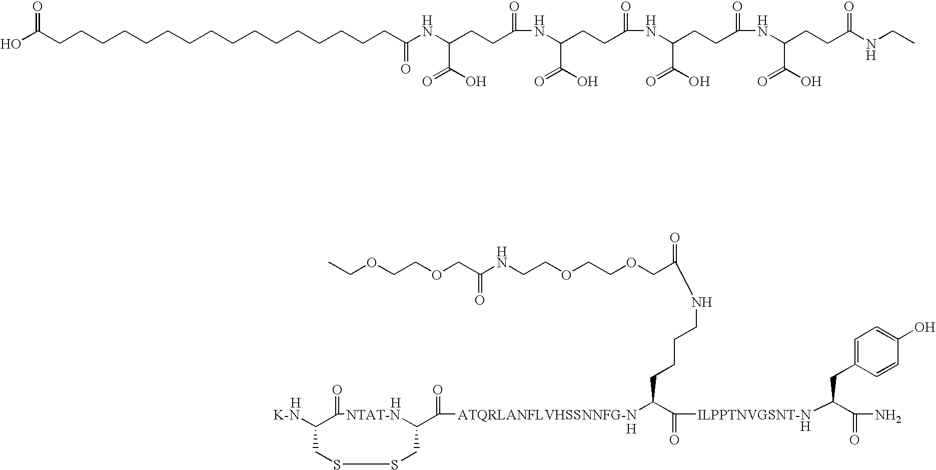

### Example 27

### N-epsilon25-[(S)-4-carboxy-4-(17-carboxyheptadecanoylamino)butyryl]-[Lys25]-pramlintide (1-37)

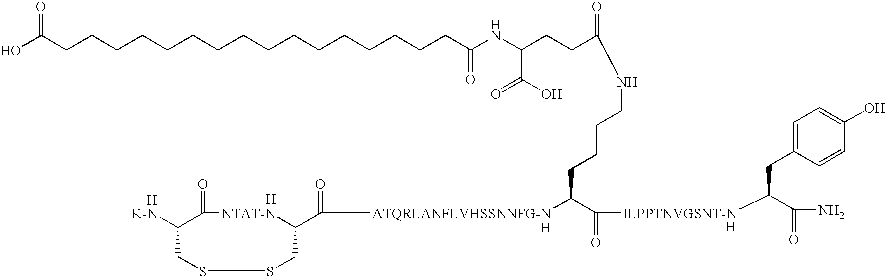

### Example 28

### N-epsilon3-[(S)-4-Carboxy-4-(17-carboxyheptadecanoylamino)butyryl]-[Lys3,Arg18,Gln21,Leu23,Val26]-pramlintide (1-37)

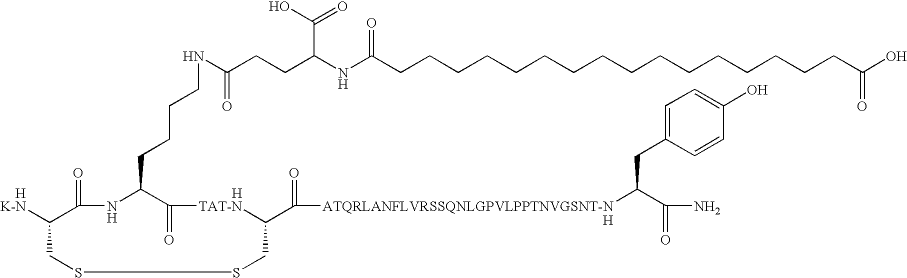

### Example 29

### N-epsilon3-(17-carboxyheptanoyl)-[Glu1,Lys3,Glu18]-pramlintide (1-37)

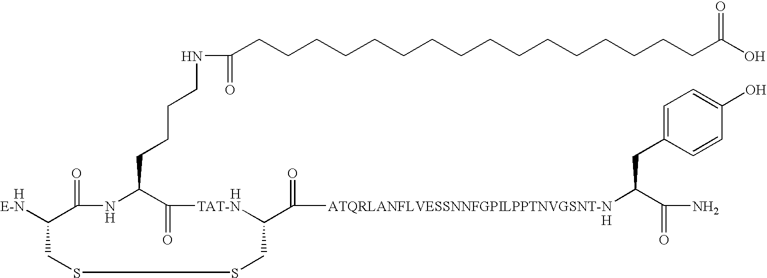

### Example 30

### N-epsilon3-[2-(2-{2-[4-(16-1H-tetrazol-5-yl-hexadecanoylsulfamoyl) butyrylamino]ethoxy}ethoxy)acetyl]-[Lys3]pramlintide (1-37)

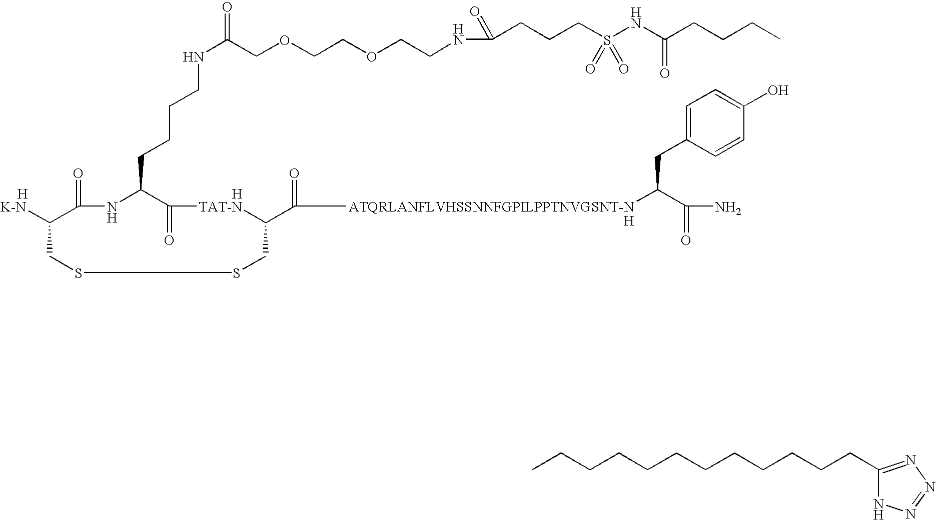

### Example 31

### N-alpha-[4-(16-1H-tetrazol-5-yl-hexadecanoylsulfamoyl)-butyryl]-Glu-Glu-Arg-[Glu1]pramlintide (1-37)

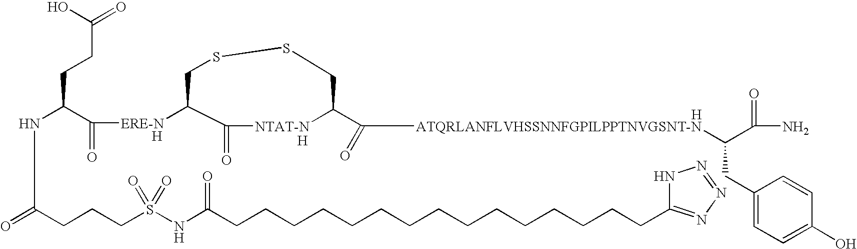

### Example 32

### N-epsilon3 (19-carboxynonadecanoyl-Glu-Glu-Arg-Glu) [Lys3, Arg18, Gln21, Leu23, Val26]pramlintide

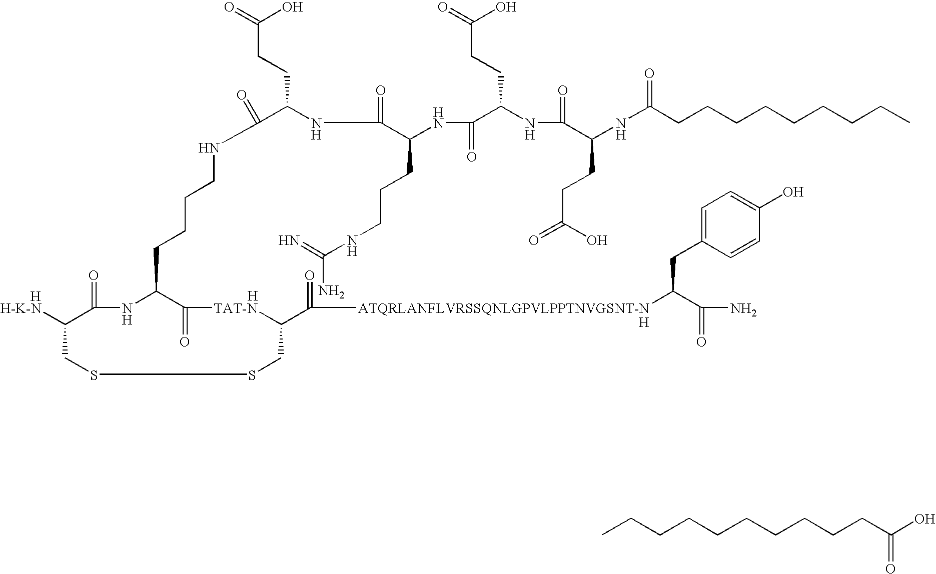

### Example 33

### N-epsilon3-(2-(2-(2-(19-carboxynonadecanoyl-Glu-Glu-amino)ethoxy)ethoxy)acetyl) [Lys3, Arg18, Gln21, Leu23, Val26]pramlintide (1-37)

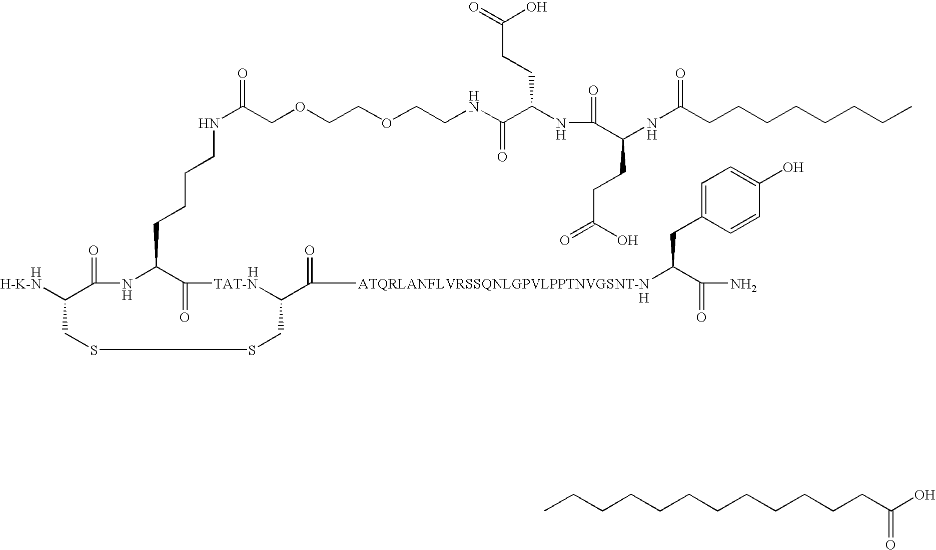

### Example 34

### N-epsilon3-(19-carboxynonadecanoyl-Glu-Glu-Glu-Glu) [Lys3, Arg18, Gln21, Leu23, Val26]pramlintide (1-37)

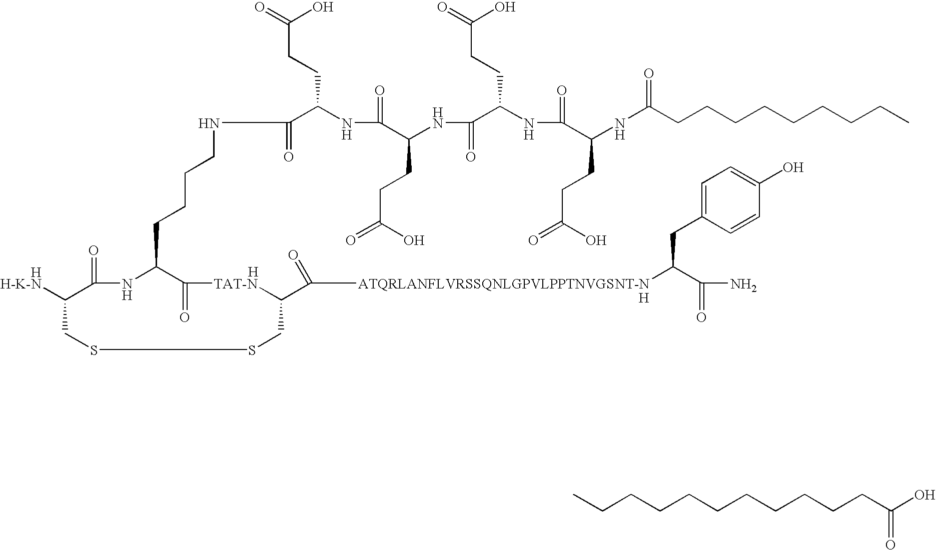

### Example 35

### N-alpha-(19-carboxynonadecanoyl)-Glu-Glu-Arg-[Glu1,Gln3,Arg18,Gln21,Leu23,Val26]-pramlintide (1-37)

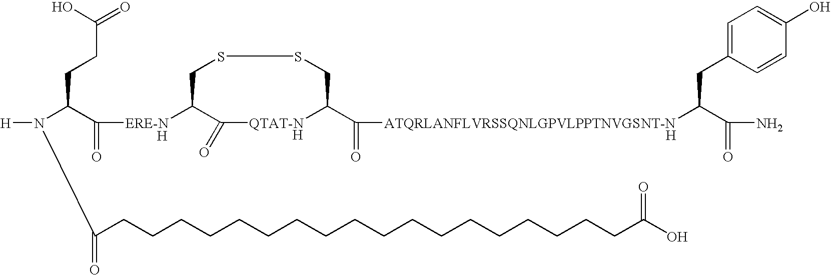

### Example 36

### N-alpha-(2-(2-(2-(19-carboxynonadecanoyl-Glu-amino)ethoxy)ethoxy)acetyl)[Glu1,Gln3,Arg18,Gln21,Leu23,Val26]-pramlintide (1-37)

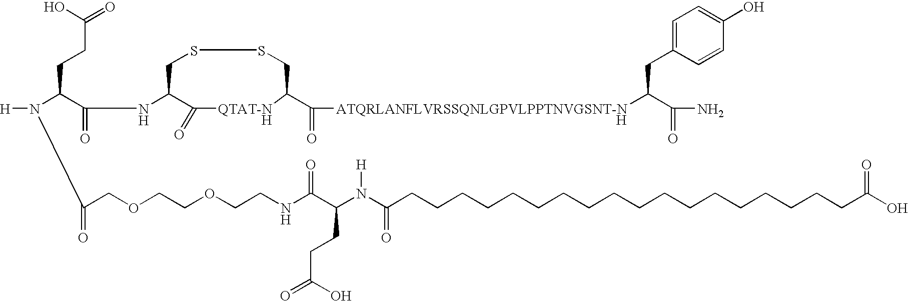

### Example 37

### N-alpha-(19-carboxynonadecanoyl)-Glu-Glu-Glu-[Glu1,Gln3,Arg18,Gln21,Leu23,Val26]-pramlintide (1-37)

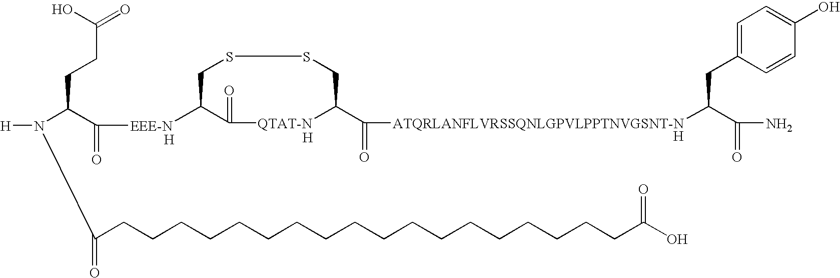

### Example 38

### N-alpha-[(S)-4-carboxy-4-(19-carboxynonadecanoylamino)butyryl]-Glu-Glu-Arg-[Glu1]-pramlintide (1-37)

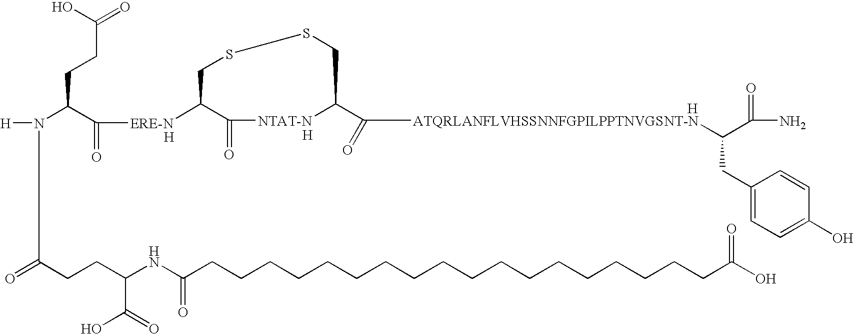

### Example 39

### N-alpha-{4-carboxy-4-[4-(16-1H-tetrazol-5-ylhexadecanoylsulfamoyl)butyrylamino]butyryl}-Glu-Glu-Arg-[Glu1]-pramlintide (1-37)

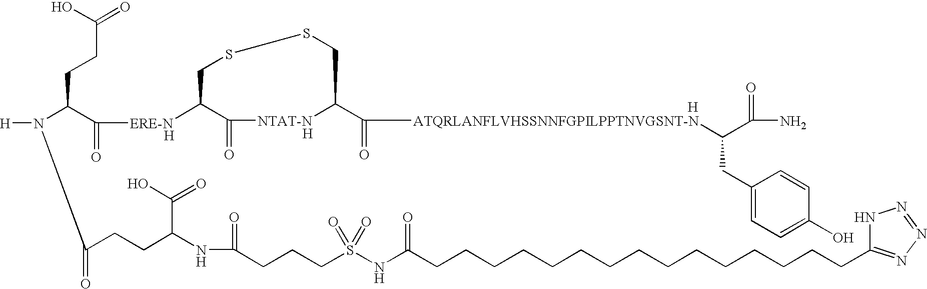

### Example 40

### N-alpha-(4-carboxy-4-{6-[4-(16-1H-tetrazol-5-yl-hexadecanoylsulfamoyl) butyrylamino]hexanoylamino}butyryl)-Glu-Glu-Arg-[Glu1]-pramlintide (1-37)

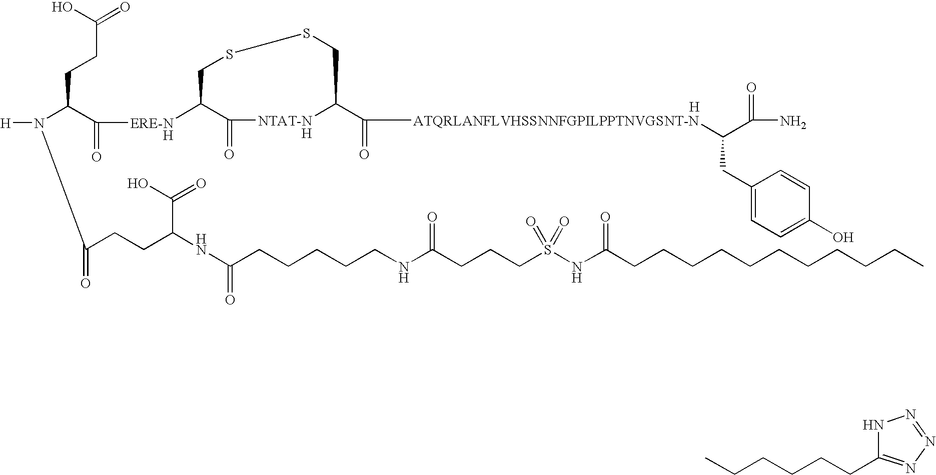

### Example 41

### N-alpha-(17-carboxyheptadecanoyl)-Glu-Glu-Arg-[Glu1]pramlintide (1-37)

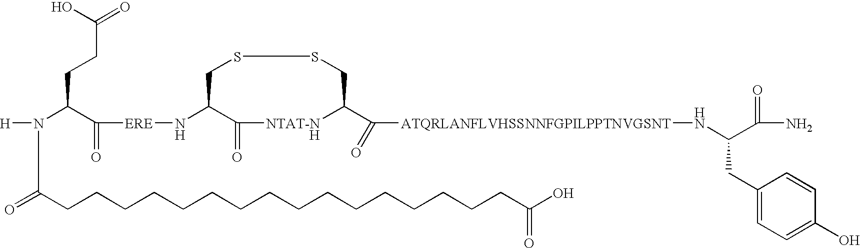

### Example 42

### N-epsilon3-{4-carboxy-4-[4-(16-1H-tetrazol-5-ylhexadecanoylsulfamoyl)butyrylamino]butyryl}-[Lys3]-pramlintide (2-37)

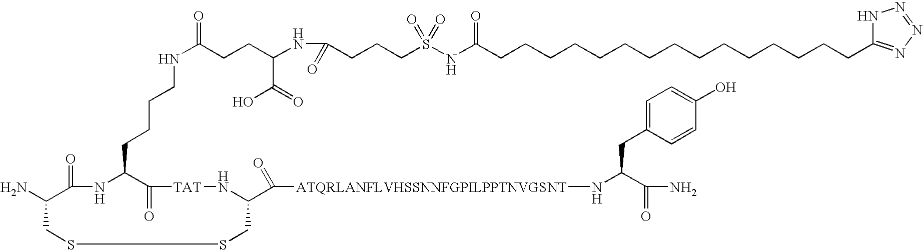

### Example 43

### N-alpha-[(S)-4-carboxy-4-(19-carboxynonadecanoylamino)butyryl]-pramlintide (2-37)

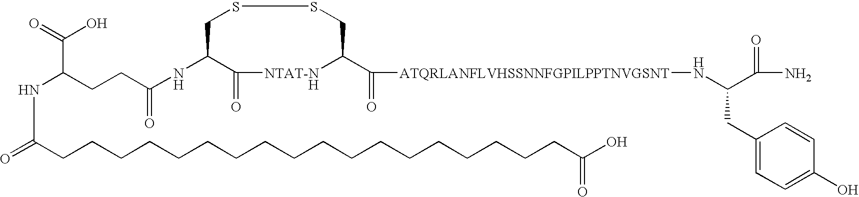

### Example 44

### N-alpha-(2-{2-[2-((S)-2-{(S)-4-carboxy-2-[(S)-4-carboxy-2-(19-carboxynonadecanoylamino)butyrylamino]butyrylamino}-5-guanidinopentanoylamino) ethoxy]ethoxy}acetyl)-[Glu1]-pramlintide (1-37)

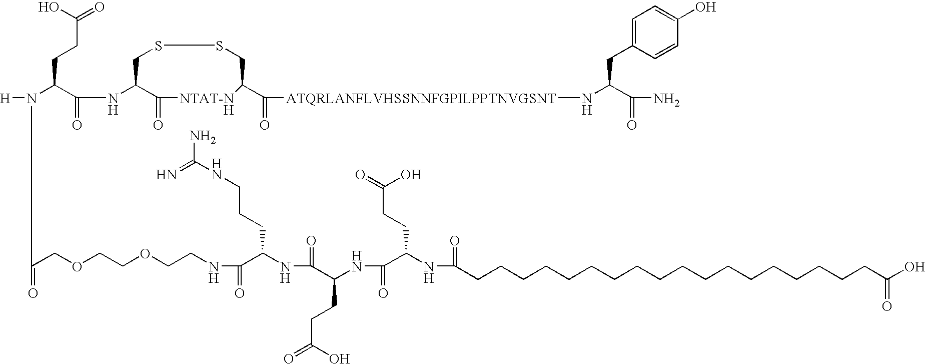

### Example 45

### N-alpha-[(S)-4-carboxy-4-(19-carboxynonadecanoylamino)butyryl]-[Arg1]-pramlintide (1-37)

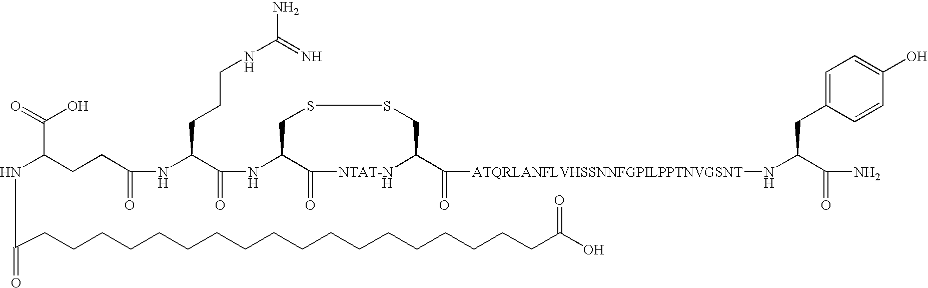

### Example 46

### N-alpha-(19-carboxynonadecanoyl)-Glu-[Arg1]-pramlintide (1-37)

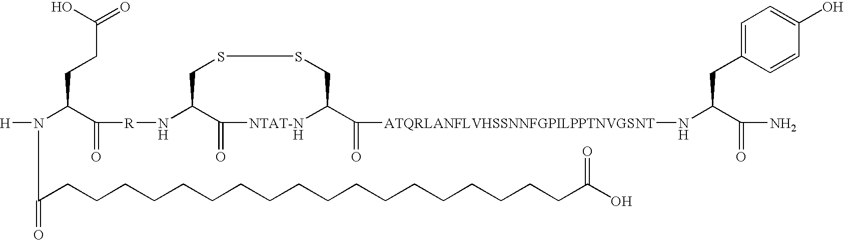

### Example 47

### N-alpha-[(S)-4-carboxy-4-(19-carboxynonadecanoylamino)butyryl]-Arg-[Arg1]-pramlintide (1-37)

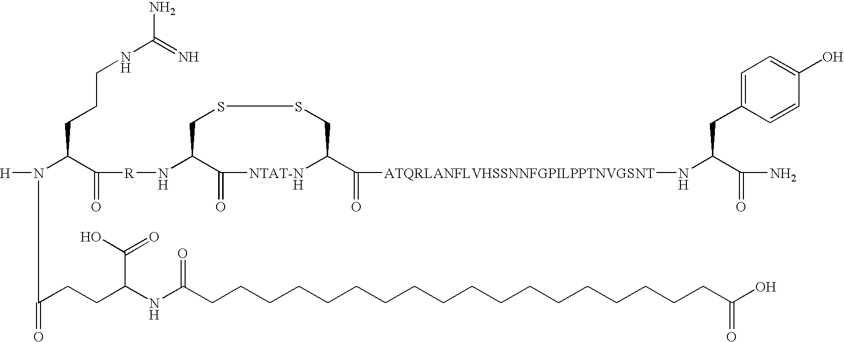

### Example 48

### N-alpha-[(S)-4-carboxy-4-(19-carboxynonadecanoylamino)butyryl]-Lys-pramlintide (1-37)

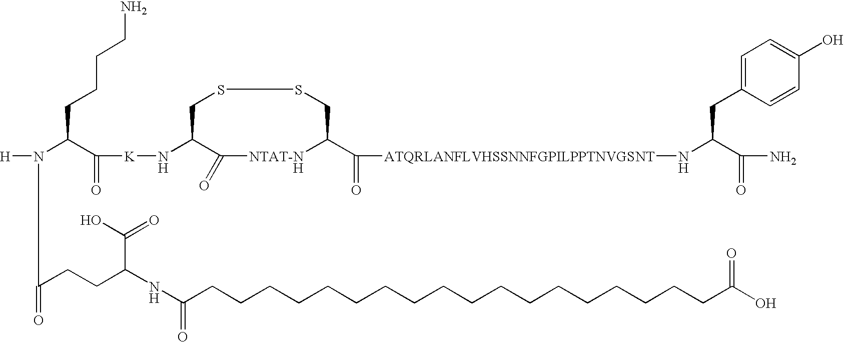

### Example 49

### N-alpha-[(S)-4-carboxy-4-(19-carboxynonadecanoylamino)butyryl]-Arg-pramlintide (1-37)

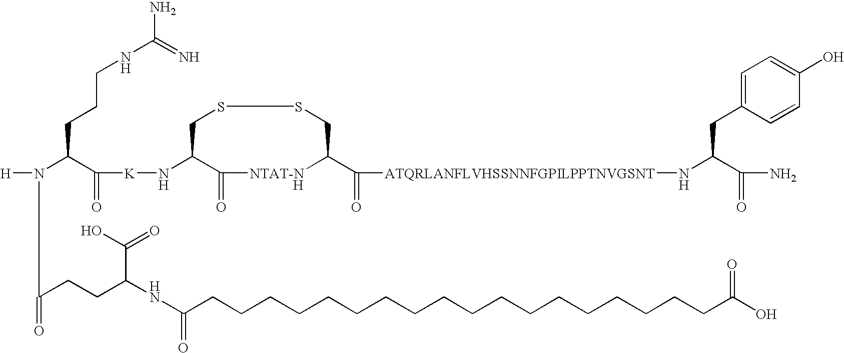

### Example 50

### N-alpha-(19-carboxynonadecanoyl)-Glu-Lys-Arg-pramlintide (1-37)

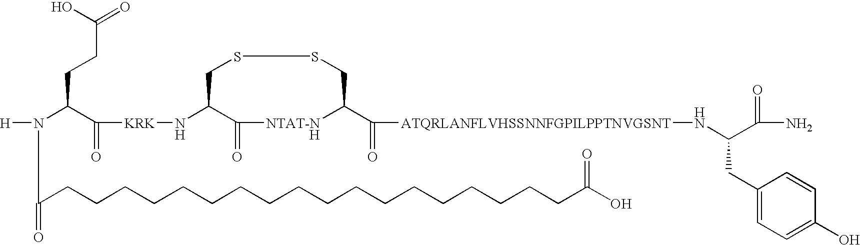

### Example 51

### N-alpha-(19-carboxynonadecanoyl)-Glu-Lys-[Arg1]-pramlintide (1-37)

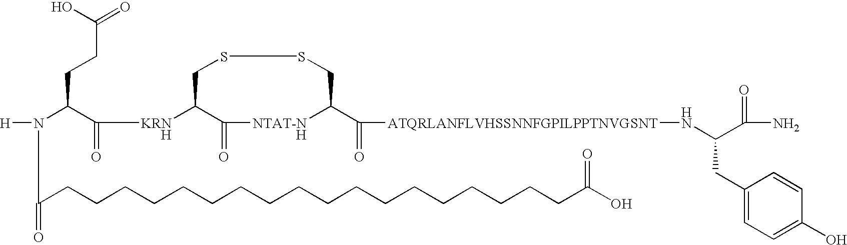

The amylin derivatives according to examples 1-51 were tested in the “Amylin luciferase assay” as described above and the results are shown in table 1. The receptor affinities of the derivatives were tested in the “Amylin receptor binding assay” as described above and the results are shown in table 1. The HPLC elution data in table 1 were measured in an analytical HPLC-system using an acetonitrile/TFA gradient from 10% to 90% over 20 minutes.

TABLE 1

| | | Potency | |
|---|---|---|---|
| | MW | luciferase assay | HPLC elution (% |
| Example No. | Confirmed by MS | pM | acetonitrile) |
| 1 | 4376.0 | 400 | 65 |
| 2 | 4260.0 | 50 | 60 |
| 3 | 4550.3 | 100 | 61 |
| 4 | 4305.0 | 5000 | 66 |
| 5 | 4277.0 | — | 64 |
| 6 | 4595.4 | 200 | 65 |
| 7 | 4578.4 | 100 | 66 |
| 8 | 4593.3 | 100 | 63 |
| 9 | 4586.3 | 40 | 62 |
| 10 | 4595.4 | 60 | 64 |
| 11 | 4679.4 | 200 | 58 |
| 12 | 4707.5 | 300 | 61 |
| 13 | 4578.4 | 60 | 62 |
| 14 | 4288.1 | 50 | 62 |
| 15 | | | |
| 16 | 4217.9 | 30 | 63 |
| 17 | 4766.4 | 900 | 61 |
| 18 | 4349.1 | 700 | 61 |
| 19 | 4639.4 | 10000 | 61 |
| 20 | 4897.6 | 20000 | 59 |
| 21 | 4265.0 | 30 | 61 |
| 22 | 4279.0 | 60 | 60 |
| 23 | 4298.9 | 70 | 60 |
| 24 | 4088.8 | 100 | 63 |
| 25 | 4689.3 | 80 | 64 |
| 26 | 5083.8 | 300 | 59 |
| 27 | 4406.1 | 90 | 62 |
| 28 | 4374.1 | 40 | 61 |
| 29 | 4419.2 | 30 | 63 |
| 30 | 4564.3 | 100 | |
| 31 | 4820.5 | 90 | |
| 32 | 4816.6 | 300 | |
| 33 | 4676.5 | 300 | |
| 34 | 4789.5 | 600 | |
| 35 | 4688.4 | 90 | |
| 36 | 4548.3 | 400 | |
| 37 | 4661.3 | 2000 | |
| 38 | 4818.5 | 300 | |
| 39 | 4949.6 | 800 | |
| 40 | 5062.7 | 2000 | |
| 41 | 4661.3 | 60 | |
| 42 | 4420.1 | 40 | |
| 43 | 4274.9 | 100 | |
| 44 | 4834.5 | 400 | |
| 45 | 4431.1 | 40 | |
| 46 | 4431.1 | 100 | |
| 47 | 4587.3 | 100 | |
| 48 | 4531.3 | 100 | |
| 49 | 4559.3 | 200 | |
| 50 | 4687.5 | 80 | |
| 51 | 4559.3 | 70 | |

### Peptide Synthesis:

One method of peptide synthesis was by Fmoc chemistry on a microwave-based Liberty peptide synthesizer (CEM Corp., North Carolina). The resin was Tentagel S RAM with a loading of 0.25 mmol/g. The coupling chemistry was DIC/HOAt in NMP using amino acid solutions of 0.3 M in NMP and a molar excess of 6-8 fold. Coupling conditions was 5 minutes at up to 70° C. Deprotection was with 5% piperidine in NMP at up to 70° C. When a chemical modification of a lysine side chain was desired, the lysine was incorporated as Lys(mtt) and the N-terminal amino acid was protected by treatment with Boc-carbonate. The mtt group was repomed by suspending the resin in neat hexafluoroisopropanol for 20 minutes followed by washing with DCM and NMP. The chemical modification of the lysine was performed either by manual synthesis or by one or more automated steps on the Liberty followed by a manual coupling. Another method of peptide synthesis was by Fmoc chemistry on an ABI 433 with HBTU coupling. After synthesis the resin was washed with DCM and dried, and the peptide was cleaved from the resin by a 2 hour treatment with TFA/TIS/water (92.5/5/2.5) followed by precipitation with diethylether. After further washing with diethylether and drying, the peptide was redissolved in water at 1-2 mg/ml, pH adjusted to about 4.5, and the disulfide bridge formed by treatment with 1 eq. of [Pt(IV) ethylenediamine₂Cl₂]Cl₂ overnight. The peptide was immediately purified by standard RP-HPLC on a C18 column using acetonitrile/TFA. Alternatively, the disulfide bridge was formed on the resin by treatment with 10 equivalents of iodine in NMP for 1 hour and in this case the peptide was purified directly after cleavage and ether precipitation. The identity of the peptide was confirmed by MALDIMS.

### Abbreviations Used:

DMF: N,N dimethylformamide
HBTU: 2-(1H-Benzotriazol-1-yl-)-1,1,3,3 tetramethyluronium hexafluorophosphate
Fmoc: 9H-fluoren-9-ylmethoxycarbonyl
Boc: tert butyloxycarbonyl
Mtt: 4-methyltrityl
DCM: dichloromethane
TIS: triisopropylsilane)
TFA: trifluoroacetic acid

### NMP: 1-Methyl-pyrrolidin-2-one

HOAt: 1-Hydroxy-7-azabenzotriazole
DIC: Diisopropylcarbodiimide

1. A derivative of human amylin with SEQ ID NO:17 or an analogue thereof,
wherein
a) the amino acid residue in position 1 is any natural amino acid which is connected to a N-terminal extension consisting of 1-10 amino acids, wherein
a. said extension is further linked to an albumin binding residue, optionally via a linker, or
b. an amino acid residue in position 2-37 has been substituted with a lysine residue, and said lysine residue is linked to an albumin binding residue, optionally via a linker;
or
b) the amino acid residue in position 3, 17 21, 25 or 29 has been substituted with a lysine residue and wherein said lysine residue is linked to an albumin binding residue, optionally via a linker;
or
c) the amino acid residue in position 18 is arginine, and an amino acid residue in position 1-17 or 19-37 has been substituted with a lysine residue, and wherein said lysine residue is linked to an albumin binding residue, optionally via a linker;
wherein the amino acid numbering conforms with the amino acid numbering in SEQ ID NO:17.

2. A derivative of human amylin with SEQ ID NO:17 or an analogue thereof according to claim 1, wherein
the amino acid residue in position 1 is any natural amino acid which is connected to a N-terminal extension consisting of 1-10 amino acids, wherein
a) said extension is further linked to an albumin binding residue, optionally via a linker
b) an amino acid residue in position 2-37 has been substituted with a lysine residue, and said lysine residue is linked to an albumin binding residue, optionally via a linker;
and the amino acid residues in position 25, position 28 and position 29 has been substituted with proline and optionally the amino acid residue in position 18 is arginine;
wherein the amino acid numbering conforms with the amino acid numbering in SEQ ID NO:17.

3. A derivative of human amylin with SEQ ID NO:17 or an analogue thereof according to claim 1, wherein
the amino acid residue in position 3, 17, 21, 25 or 29 has been substituted with a lysine residue and wherein said lysine residue is linked to an albumin binding residue, optionally via a linker and the amino acid residues in position 25, position 28 and position 29 has been substituted with proline and optionally the amino acid residue in position 18 is arginine;
wherein the amino acid numbering conforms with the amino acid numbering in SEQ ID NO:17.

4. The derivative according to claim 1, wherein the derivative has from 1-12 amino acid substitutions compared to human amylin.

5. The derivative according to claim 1, wherein the derivative is an analogue of human amylin of SEQ ID NO:17, wherein 0-8 additional charges have been added compared to human amylin.

6. A derivative according to claim 1 comprising an amino acid sequence of formula 1:
(SEQ ID No: 18)
Formula (1)
Xaa₁-Cys-Xaa₃-Thr-Ala-Thr-Cys-Ala-Thr-Gln-Arg-Leu-
Ala-Asn-Phe-Leu-Xaa₁₇-Xaa₁₈-Ser-Ser-Xaa₂₁-Asn-
Xaa₂₃-Gly-Xaa₂₅-Xaa₂₆-Leu-Xaa₂₈-Xaa₂₉-Thr-Asn-Val-
Gly-Ser-Asn-Thr-Tyr-Xaa₃₈
wherein
Xaa₁ is deleted or independently selected from Lys, Arg and Glu;
Xaa₃ is independently selected from Asn, Gly, Gln and Lys;
Xaa₇ is independently selected from Ala, Val and Lys;
Xaa₁₈ is independently selected from His, Pro and Arg;
Xaa₂₃ is independently selected from Phe and Leu;
Xaa₂₁ is independently selected from Asn, Gln and Lys;
Xaa₂₅ is independently selected from Ala, Pro and Lys;
Xaa₂₆ is independently selected from Val and Ile;
Xaa₂₈ is independently selected from Ser and Pro;
Xaa₂₉ is independently selected from Ser, Pro and Lys;
the C-terminal may optionally be derivatized as an amide;
the N-terminal may optionally be extended with 1-10 amino acid residues
wherein the amino acid residue Xaa₁ is connected to a N-terminal extension consisting of 1-10 amino acids, which extension can be further linked to an albumin binding residue, optionally via a linker, or
the amino acid residue Xaa₁, Xaa₃, Xaa₁₇, Xaa₂₁, Xaa₂₅ or Xaa₂₉ is lysine, and said lysine residue is linked to an albumin binding residue, optionally via a linker;
wherein if Xaa₁ is Lys and Xaa₃ is Asn and Xaa₂₁ is Asn and Xaa₂₅ is Ala or Pro, then Xaa₂₉ is Lys; and
if Xaa₁ is Glu or Arg, then said Glu or Arg is connected to a N-terminal extension consisting of 1-10 amino acids, and said extension is further linked to an albumin binding residue; and
if Xaa₁ is Lys, then one amino acid in a position selected from the group consisting of Xaa₁, Xaa₃, Xaa₂₁, Xaa₂₅ and Xaa₂₉ in formula (I) is linked to an albumin binding residue, optionally via a linker.

7. A derivative according to claim 6, wherein the albumin binding residue is a carboxylic acid or 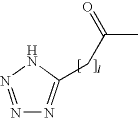
where 1 is 12, 13, 14, 15, 16, 17, 18, 19 or 20.

8. A derivative according to claim 7, wherein the albumin binding residue via a linker is connected via the ε-amino group of a lysine residue and wherein the linker is selected from the group consisting of one or more units of ethylene glycol, 1-4 amino acid residues and

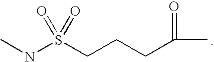

9. A pharmaceutical composition comprising a derivative according to claim 1, and a pharmaceutically acceptable excipient.

10. (canceled)

11. A method for the treatment of hyperglycemia, type 2 diabetes, impaired glucose tolerance, type 1 diabetes, obesity, syndrome X or dyslipidemia in a subject in need of such treatment, the method comprising administering to the subject a pharmaceutical composition according to claim 9.

12. A process for preparing a pharmaceutical composition according to claim 9 comprising mixing a derivative according to claim 1 with pharmaceutically acceptable substances and/or excipients.

13. A method for increasing the time of action in a patient of human amylin with SEQ ID NO:17 or an analog thereof, the method comprising modifying
a) the amino acid residue in position 1 by optionally substituting with any natural amino acid, and by connecting said natural amino acid to a N-terminal extension consisting of 1-10 amino acids, and further
a. linking said extension to an albumin binding residue, optionally via a linker, or
b. modifying an amino acid residue in position 2-37 by substitution with a lysine residue, and by linking said lysine residue to an albumin binding residue, optionally via a linker,
or;
b) the amino acid residue in position 3, 17, 21, 25 or 29 by substitution with a lysine residue and by linking said lysine residue to an albumin binding residue, optionally via a linker,
or;
c) the amino acid residue in position 18 by substitution with an arginine residue and an amino acid residue in position 2-17 or 19-37 by substitution with a lysine residue and by linking said lysine residue to an albumin binding residue, optionally via a linker;
d) the amino acid in position 18 is arginine, the amino acids in position 25, position 28 and position 29 are proline, and an amino acid residue in position 1-17 or 19-37 has been substituted with a lysine residue, and wherein such a lysine residue is linked to an albumin binding residue, optionally via a linker
wherein the amino acid numbering conforms with the amino acid numbering in SEQ ID NO:17.


SEQUENCE LISTING (32 sequences)

SEQ ID NO: 1 - protein, 37 aa, Artificial Sequence
  Synthetic
KCNTATCATQRLANFLVHSSNNFGPILPPTNVGSNTY

SEQ ID NO: 2 - protein, 37 aa, Artificial Sequence
  Synthetic
KCKTATCATQRLANFLVHSSNNFGPILPPTNVGSNTY

SEQ ID NO: 3 - protein, 37 aa, Artificial Sequence
  Synthetic
KCNTATCATQRLANFLVHSSNNFGPILPKTNVGSNTY

SEQ ID NO: 4 - protein, 37 aa, Artificial Sequence
  Synthetic
KCNTATCATQRLANFLVHSSKNFGPILPPTNVGSNTY

SEQ ID NO: 5 - protein, 37 aa, Artificial Sequence
  Synthetic
KCNTATCATQRLANFLKHSSNNFGPILPPTNVGSNTY

SEQ ID NO: 6 - protein, 37 aa, Artificial Sequence
  Synthetic
KCNTATCATQRLANFLVHSSNNFGKILPPTNVGSNTY

SEQ ID NO: 7 - protein, 37 aa, Artificial Sequence
  Synthetic
KCNTATCATQRLANFLVRSSNNFGKILPPTNVGSNTY

SEQ ID NO: 8 - protein, 36 aa, Artificial Sequence
  Synthetic
CNTATCATQRLANFLVRSSNNLGPVLPPTNVGSNTY

SEQ ID NO: 9 - protein, 37 aa, Artificial Sequence
  Synthetic
KCKTATCATQRLANFLVPSSNNFGPILPPTNVGSNTY

SEQ ID NO: 10 - protein, 37 aa, Artificial Sequence
  Synthetic
KCNTATCATQRLANFLVRSSNNFGPILPPTNVGSNTY

SEQ ID NO: 11 - protein, 37 aa, Artificial Sequence
  Synthetic
KCKTATCATQRLANFLVRSSNNFGPILPPTNVGSNTY

SEQ ID NO: 12 - protein, 37 aa, Artificial Sequence
  Synthetic
KCGTATCATQRLANFLARSSNNFGPILSPTNVGSNTY

SEQ ID NO: 13 - protein, 40 aa, Artificial Sequence
  Synthetic
EERECNTATCATQRLANFLVHSSNNFGPILPPTNVGSNTY

SEQ ID NO: 14 - protein, 37 aa, Artificial Sequence
  Synthetic
KCKTATCATQRLANFLVRSSQNLGPVLPPTNVGSNTY

SEQ ID NO: 15 - protein, 37 aa, Artificial Sequence
  Synthetic
ECKTATCATQRLANFLVHSSNNFGPILPPTNVGSNTY

SEQ ID NO: 16 - protein, 38 aa, Artificial Sequence
  Synthetic
EECNTATCATQRLANFLVHSSNNFGPILPPTNVGSNTY

SEQ ID NO: 17 - protein, 37 aa, Homo Sapiens [sequence omitted]

SEQ ID NO: 18 - protein, 37 aa, Artificial Sequence
  Synthetic
XCXTATCATQRLANFLXXSSXNXGXXLXXTNVGSNTY

SEQ ID NO: 19 - protein, 40 aa, Artificial Sequence
  Synthetic
EERECQTATCATQRLANFLVRSSQNLGPVLPPTNVGSNTY

SEQ ID NO: 20 - protein, 40 aa, Artificial Sequence
  Synthetic
EEEECQTATCATQRLANFLVRSSQNLGPVLPPTNVGSNTY

SEQ ID NO: 21 - protein, 36 aa, Artificial Sequence
  Synthetic
CKTATCATQRLANFLVHSSNNFGPILPPTNVGSNTY

SEQ ID NO: 22 - protein, 36 aa, Artificial Sequence
  Synthetic
CNTATCATQRLANFLVHSSNNFGPILPPTNVGSNTY

SEQ ID NO: 23 - protein, 37 aa, Artificial Sequence
  Synthetic
ECNTATCATQRLANFLVHSSNNFGPILPPTNVGSNTY

SEQ ID NO: 24 - protein, 37 aa, Artificial Sequence
  Synthetic
RCNTATCATQRLANFLVHSSNNFGPILPPTNVGSNTY

SEQ ID NO: 25 - protein, 38 aa, Artificial Sequence
  Synthetic
ERCNTATCATQRLANFLVHSSNNFGPILPPTNVGSNTY

SEQ ID NO: 26 - protein, 38 aa, Artificial Sequence
  Synthetic
RRCNTATCATQRLANFLVHSSNNFGPILPPTNVGSNTY

SEQ ID NO: 27 - protein, 38 aa, Artificial Sequence
  Synthetic
KKCNTATCATQRLANFLVHSSNNFGPILPPTNVGSNTY

SEQ ID NO: 28 - protein, 38 aa, Artificial Sequence
  Synthetic
RKCNTATCATQRLANFLVHSSNNFGPILPPTNVGSNTY

SEQ ID NO: 29 - protein, 40 aa, Artificial Sequence
  Synthetic
EKRKCNTATCATQRLANFLVHSSNNFGPILPPTNVGSNTY

SEQ ID NO: 30 - protein, 39 aa, Artificial Sequence
  Synthetic
EKRCNTATCATQRLANFLVHSSNNFGPILPPTNVGSNTY

SEQ ID NO: 31 - protein, 40 aa, Artificial Sequence
  Synthetic
EERECKTATCATQRLANFLVRSSQNLGPVLPPTNVGSNTY

SEQ ID NO: 32 - protein, 37 aa, Artificial Sequence
  Synthetic
ECQTATCATQRLANFLVRSSQNLGPVLPPTNVGSNTY